(19) United States
(12) Patent Application Publication
(10) Pub. No.: US 2019/0284149 A1
(43) Pub. Date: Sep. 19, 2019

(54) ARYL HYDROCARBON RECEPTOR MODULATOR

(71) Applicant: ARIAGEN, INC., Menlo Park, CA (US)

(72) Inventors: Jiasheng Song, Madison, WI (US); Suoming Zhang, Shanghai (CN); Guodong Li, Shanghai (CN); Luqing Yang, Shanghai (CN)

(73) Assignee: ARIAGEN, INC., Menlo Park, CA (US)

(21) Appl. No.: 16/229,586

(22) Filed: Dec. 21, 2018

Related U.S. Application Data
(63) Continuation-in-part of application No. 15/998,528, filed on Aug. 16, 2018.
Application No. 15/998,528 is a continuation of application No. 15/509,722, filed on Mar. 8, 2017, now Pat. No. 10,081,610.
(60) Provisional application No. 62/049,804, filed on Sep. 12, 2014.

(51) Int. Cl. C07D 277/20; C07D 209/12; C07D 417/06

(52) U.S. Cl. CPC C07D 277/20; C07C 67/47; C07D 417/06; C07D 209/12

(57) ABSTRACT

Disclosed is an aryl hydrocarbon receptor modulator of formula (I), and a pharmaceutically acceptable salt thereof, wherein

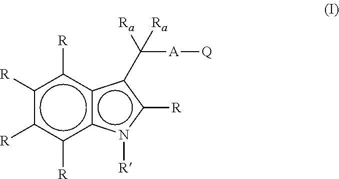

R′ is H, CN, CH₂(OH)R₀, CₘH₂ₘ₊₁, CₙH₂ₙ₋₁, CₙH₂ₙ₋₃,

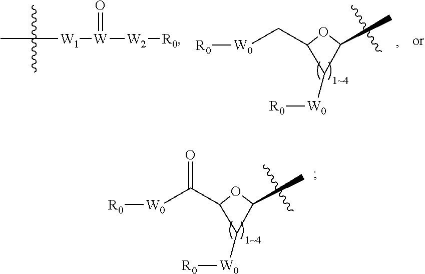

two Rₐ are independently H or two Rₐ together form =0 or ═N—W₃—R₁; A is a C₆ to C₁₀ aromatic ring unsubstituted or substituted with 1 to 3 R, or a C₂-C₁₀ heteroaromatic ring interrupted by 1 to 5 heteroatoms selected from N, 0, and S or a 4 to 7 membered nonaromatic heterocyclic ring containing C═N and interrupted by 1 to 3 heteroatoms selected from N, 0, and S, with either one unsubstituted or substituted with 1 to 3 R; Q is R, or is a C₆ to C₁₀ aromatic ring or a C₂ to C₁₀ heteroaromatic ring unsubstituted or substituted with 1 to 3 R and interrupted by 1 to 5 heteroatoms selected from N, 0, and S; and R is R_{C} which is C-attached or R_{N} which is N-attached. The compounds of formula (I) of the present invention can regulate AhR activity, and can be used to inhibit the growth of cancer cells and inhibit the metastasis and invasion of tumor cells.

### TECHNICAL FIELD

The present invention belongs to the technical field of anti-tumor compounds and relates to a class of compounds capable of modulating aryl hydrocarbon receptor (AhR) activity and pharmaceutically acceptable salts thereof.

### BACKGROUND

Due to changes in the environment and lifestyle, the increasing cases of cancers, combined with its high mortality rate, poses a serious threat to human health. Although significant progress has been made in the medical treatment of certain cancers and targeting drugs and immunotherapy have significantly increased the survival rate of patients, the overall 5-year survival rate for all cancer patients is only increased by 10% in the past 20 years. Moreover, the discovery and treatment of cancers is extremely difficult because of drug resistance or uncontrolled metastasis and rapid growth of malignant tumors.

Aryl hydrocarbon receptor (AhR) is a class of intracellular transcriptional regulators that can be susceptible to xenobiotic stimuli from the ambient environment and mediate toxic responses. Activated AhR can regulate expression of genes on many chromosomes and promote decomposition of xenobiotics. Previous studies have found that the signal is also involved in some important biological processes, such as signal transduction, cell differentiation, cell apoptosis, and the like. The relationship between AhR and immune regulation has also been the focus of research; previous studies have shown that AhR can be involved in the differentiation and functions of T cells, macrophages, and DC. In addition, AhR also plays a key role in immune rejection after organ transplantation. It has been found that using Dioxin to activate AhR in mice can reduce the survival rate of mice after virus infection. The rate of differentiation and proliferation of virus-specific CD8 T cells is also affected. For another example, in the compounds listed below, DIM and its derivatives have tumor inhibitory activity (Breast Cancer Res. Treat. 2001, 66, 147); and DIM is currently in phase II clinical study for the treatment of prostate and cervical cancers. Both natural products ICZ and FICZ are AhR agonists that are anti-asthmatic (Chem. Rev., 2002, 102, 4303; Chem. Rev., 2012, 112, 3193; J. Biol. chem. 2009, 284, 2690). Malassezin (Bioorg. Med. Chem. 2001, 9, 955). Aminoflavonone is developed by NCI and is in phase I clinical study. Indole-3-carbinol is in phase II clinical study, and is used as chemical protective agent and immune stimulant. Phortress is an AhR agonist developed by Pharminox Univ. of Nottingham, and is used for treatment of solid TUMORS in phase I clinical study (BR. J. Cancer, 2003, 88, 599; Mol. Cancer Ther. 2004, 3, 1565). Tanshinone I is a natural AhR ligand for anti-tumor chemical protectants (Toxicol Appl Pharmacol. 2011 Apr. 1; 252(1): 18-27). 2-(indol-3-acetyl)furan (Food Chem. 2011, 127, 1764-1772). ITE, a native endogenous AhR activator, has the effect of anti-liver cancer, prostate cancer, breast cancer, and ovarian cancer (Proc. Natl. Acad. Sci. 2002, 99, 14694-9; CN102573470; WO2016040553).

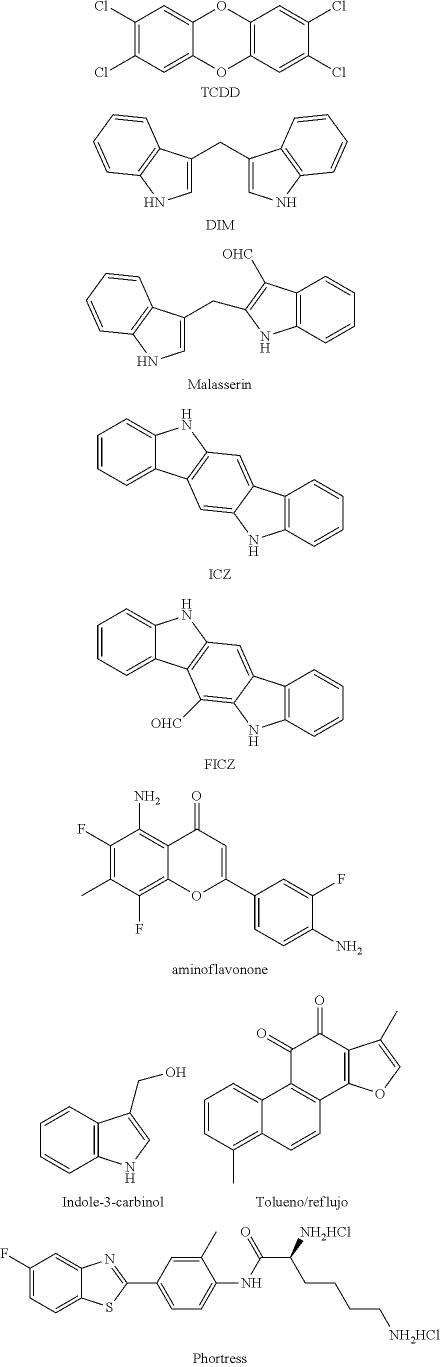

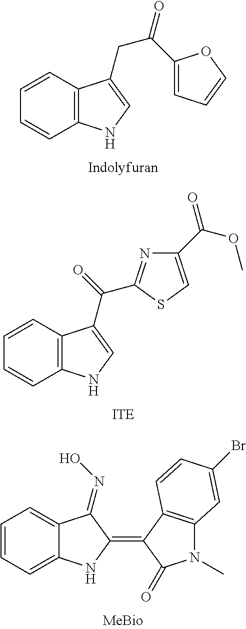 [SMILES: O=C(Cc1c[nH]c2ccccc12)c1ccco1 ; COC(=O)c1csc(C(=O)c2c[nH]c3ccccc23)n1 ; CN1C(=O)/C(=C2\Nc3ccccc3\C2=N/O)c2ccc(Br)cc21]

### SUMMARY

It is an object of the present invention to provide a new aryl hydrocarbon receptor modulator of formula (I) with AhR activity, and a pharmaceutically acceptable salt thereof,

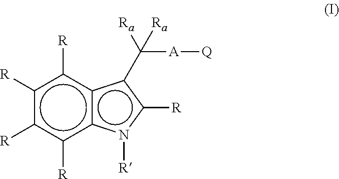

R′ is H, CH, CH₂(OH)R₀, CₘH₂ₘ₊₁, CₙH₂ₙ₋₁, CₙH₂ₙ₋₃,

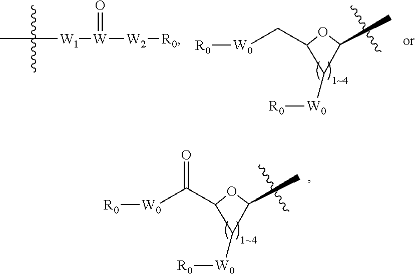

where W₀ is O or NH; W₁ is a linker bond, C(R₀)₂, C(R₀)₂O, C(R₀)₂OC(R₀)₂, or C(R₀)₂OC(R₀)₂C(R₀)₂; when W is C, S, or S(O), W₂ is a linker bond, O, NR₀, CH(N(R₀)₂), or OCH₂C(O); when W is P(OR₀), W₂ is O or NR₀; each R₀ is independently H, CₘH₂ₘ₊₁, CₘH₂ₘ₊₁OC(O), CₘH₂ₘ₊₁₋ᵣXᵣ, CₘH₂ₘ₊₁OC(O)CₘH₂ₘ, (cyclic C₄H₈NO)CₘH₂ₘ, CH₃(OCH₂CH₂)ᵤ, or CH₃(OCH₂CH₂)ᵤOCH₂;
two Rₐ are independently H or two Rₐ together form ═O, ═N—CN, or ═N—W₃—R₁, W₃ is O or NH, R₁ is H, CₘH₂ₘ₊₁, CₘH₂ₘ₊₁C(O), CₘH₂ₘ₊₁OC(O), or CₘH₂ₘ₊₁S(O)₁₋₂;
A is a C₆ to C₁₀ aromatic ring unsubstituted or substituted with 1 to 3 R, or a C₂-C₁₀ heteroaromatic ring interrupted by 1 to 5 heteroatoms selected from N, 0, and S or a 4 to 7 membered nonaromatic heterocyclic ring containing C═N and interrupted by 1 to 3 heteroatoms selected from N, 0, and S, with either one unsubstituted or substituted with 1 to 3 R;
Q is R, or a C₆ to C₁₀ aromatic ring unsubstituted or substituted with 1 to 3 R, or a 3 to 10 membered, preferably 4 to 7 membered, more preferably 5 to 6 membered heterocyclic ring, preferably heteroaromatic ring unsubstituted or substituted with 1 to 3 R, interrupted by 1 to 5, preferably 1 to 3, more preferably 2 to 3 heteroatoms selected from N, 0, and S;
R is R_{C} which is C-attached or R_{N} which is N-attached, where each R_{C} is independently X, CN, R″, —Y—OR″, —Y—C(O)R″, —Y—OC(O)R″, —Y—C(O)OR″, —Y—OC(O)OR″, —Y—NR″₂, —Y—C(O)NR″₂, —Y—NR″C(O)R″, —Y—NR″C(O)NR″₂, —Y—OC(O)NR″₂, —Y—NR″C(O)OR″, —Y—S(O)₁₋₂R″, —Y—S(O)₁₋₂NR″₂, or —Y—NR″S(O)₁₋₂R″; each R_{N} is independently CN, R″, —Y—OR″, —Y—C(O)R″, —Y—OC(O)R″, —Y—C(O)OR″, —Y—OC(O)OR″, —Y—NR″₂, —Y—C(O)NR″₂, —Y—NR″C(O)R″, —Y—NR″C(O)NR″₂, —Y—OC(O)NR″₂, —Y—NR″C(O)OR″, —Y—S(O)₁₋₂R″, —Y—S(O)₁₋₂NR″₂, or —Y—NR″S(O)₁₋₂R″;
R″ is H, D, CₘH₂ₘ₊₁, CₙH₂ₙ₋₁, CₙH₂ₙ₋₃, CₘH₂ₘ₊₁₋ᵣXᵣ, CₙH₂ₙ₋₁₋ₛXₛ, or CH₂ₙ₋₃₋ₜXₜ;
Y is a linker bond, —CₘH₂ₘ—, —CₙH₂ₙ₋₂—, —CₙH₂₋ₙ₋₄—, —CₘH₂ₘ₋ᵢXⱼ—, —CₙH₂ₙ₋₂ⱼXⱼ—, or —CₙH₂ₙ₋₄₋ₖXₖ—;
m=1 to 8, n=2 to 8, u=1 to 5, r≤2m+1, s≤2n−l, t≤2n−3, i≤2m, j≤2n−2, k≤2n−4, and X is halogen; preferably, m=1 to 5, more preferably 1 to 3; N=2 TO 6, more preferably 2 to 4; u=1 to 4, more preferably 1 to 3; and X is F, Cl, or Br.

Cyclic C₄H₈NO in the (cyclic C₄H₈NO)CₘH₂ₘ is a 6 membered ring with N and O being in meta or para position, preferably N substituted morpholine.

CₘH₂ₘ₊₁, CₘH₂ₘ₊₁₋ᵣXᵣ, —CₘH₂ₘ— and —CₘH₂ₘ₋ᵢXᵢ— may be a linear or branched saturated alkyl; CₙH₂ₙ₋₁, CₙH₂ₙ₋₁₋ₛXₛ, —CH₂H₂ₙ₋₂— and —CₙH₂ₙ₋₂₋ⱼXⱼ— may be a linear or branched alkenyl; and CₙH₂ₙ₋₃, CₙH₂ₙ₋₃₋ₜX, —CₙH₂ₙ₋₄— and —CₙH₂ₙ₋₄₋ₖXₖ may be a linear or branched alkynyl.

When n=3 to 8, CₙH₂ₙ₋₁, CₙH₂ₙ₋ᵢ₋ₛXₛ, —CₙH₂ₙ₋₂—, and —CₙH₂ₙ₋₂₋ⱼXⱼ— may also be cycloalkyl. When n=5 to 8, CₙH₂ₙ₋₃, CₙH₂ₙ₋₃₋ₜXₜ, —CₙH₂ₙ₋₄—, and —CₙH₂ₙ₋₄₋ₖXₖ may also be dialkenyl or cycloalkenyl.

In some preferred embodiments of the present invention, A is

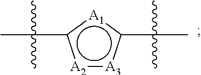

and in which case, formula (I) becomes formula (II),

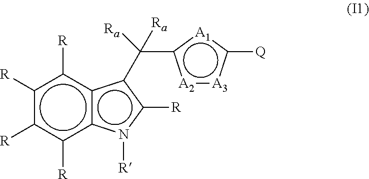

in formula (I1), one of A₁, A₂, and A₃ is O, S, or N(R) and the other two are each independently C(R) or N, which specifically can be divided into three situations: if A₁ is O, S, or N(R), A₂ and A₃ are each independently C(R) or N; if A₂ is O, S, or N(R), A₁ and A₃ are each independently C(R) or N; and if A₃ is O, S, or N(R), A₁ and A₂ are each independently C(R) or N.

In formula (I1) of the present invention, it is further preferred that one of A₁, A₂ and A₃ is O, S, or N(R) and the other two are each independently N; and in which case, A₁, A₂, and A₃ are all heteroatoms. Even further preferably, A₃ is constantly N; and in which case, formula (I1) becomes formula (Ia),

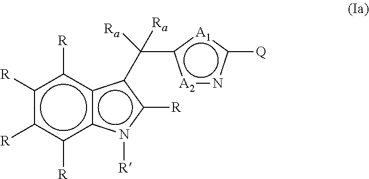

in formula (Ia), A₁ is O, S, or N(R), and A₂ is N; or A₂ is O, S, or N(R), and A₁ is N. In formula (I1) of the present invention, it may be further preferred that A₂ is CH; and in which case, formula (I1) becomes formula (Ib),

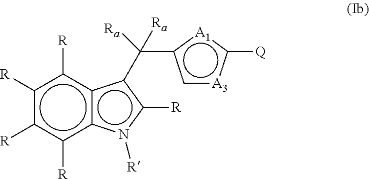

in formula (Ib), A₁ is N or C(R), and A₃ is O, S, or N(R); or At is O, S, or N(R), and A₃ is N or C(R).

In formula (I1) of the present invention, it may be further preferred that A₁ is N, A₃ is C(R), and two Rₐ together form=N—W₃—R₁ or are independently H; and in which case, formula (I1) becomes formula (Ic) or (Id),

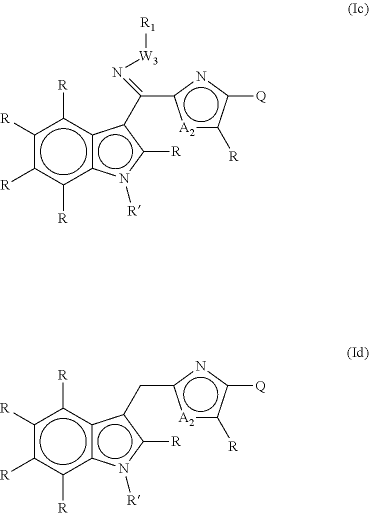

in formulas (Ic) and (Id), A₂ is O, S, or N(R).

In formula (I1) of the present invention, it may be further preferred that A₁ is N, A₃ is C(R), and R′ is

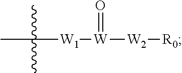

and in which case, formula (I1) becomes formula (Ie),

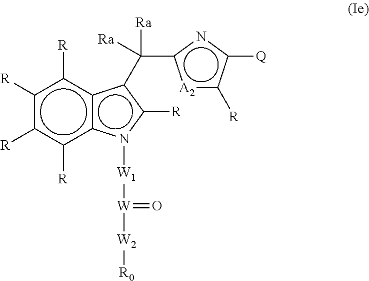

in formula (Ie), A₂ is O, S, or N(R).

In formula (I1) of the present invention, it may be further preferred that A₁ is N, A₃ is C(R), and R′ is

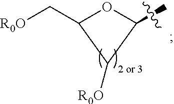

and in which case, formula (I1) becomes formula (If),

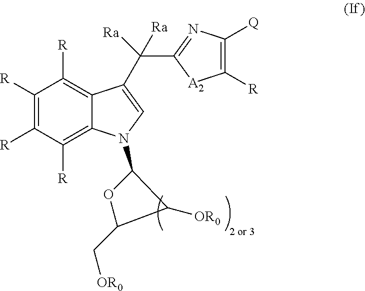

in formula (If), A₂ is O, S, or N(R), and each R₀ is independently H or Ac.

In some preferred embodiments of the present invention, Q is

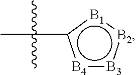

one of B₁, B₂, B₃, and B₄ is O, S, or N(R), and the other three are each independently C(R) or N; that is,
B₁ is O, S, or N(R), and B₂, B₃, and B₄ are each independently C(R) or N;
or B₂ is O, S, or N(R), and B₁, B₃, and B₄ are each independently C(R) or N;
or B₃ is O, S, or N(R), and B₁, B₂, and B₄ are each independently C(R) or N;
or B₄ is O, S, or N(R), and B₁, B₂, and B₃ are each independently C(R) or N.

In some preferred embodiments of the present invention, Q is

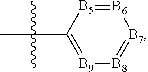

and B₅ to B₉ are C(R), i.e., Q being a benzene ring; or one or two of B₅ to B₉ are N and the others are each independently C(R), i.e.,
Q may also be a pyridine ring; and in which case, B₅ is N, and B₆ to B₉ are each independently C(R); or B₆ is N, and B₅ and B₇ to B₉ are each independently C(R); or B₇ is N, and B₅, B₆, B₈, and B₉ are each independently C(R);
Q may also be a pyridazine ring; and in which case, B₅ and B₆ are N, and B₇ to B₉ are each independently C(R); or B₆ and B₇ are N, and B₅, B₈, and B₉ are each independently C(R);
Q may also be a pyrimidine ring; and in which case, B₅ and B₇ are N, and B₆, B₈, and B₉ are each independently C(R);
Q may also be a pyrazine ring; and in which case, B₅ and B₈ are N, and B₆, B₇, and B₉ are each independently C(R).

In formula (I1) of the present invention, it may be further preferred that A₁ is N, A₂ is S, A₃ is CH, and Q is a 5 membered heteroaromatic ring; and in which case, formula (I) becomes formula (Ig),

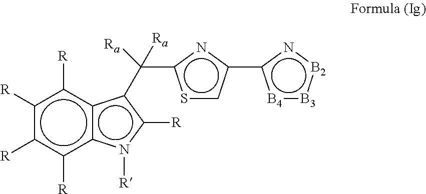

where one of B₂, B₃, and B₄ is O, S, or N(R), and the others are C(R) or N; that is,
if B₂ is O, S, or N(R), B₃ and B₄ are each independently C(R) or N;
if B₃ is O, S, or N(R), B₂ and B₄ are each independently C(R) or N;
if B₄ is O, S, or N(R), B₂ and B₃ are each independently C(R) or N.

In formula (II) of the present invention, it may be further preferred that A₁ is N, A₂ is S, A₃ is CH, and Q is a 5 membered heterocyclic ring; and in which case, formula (I) becomes formula (Ih),

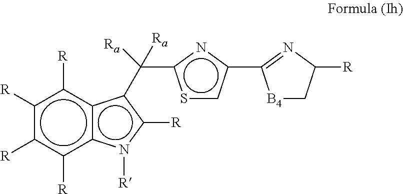

### B₄ is O, S, or N(R).

In some preferred embodiments of the present invention, A is a nonaromatic heterocyclic ring interrupted by N and S, and Q is R; and in which case, formula (I) becomes formula (I2),

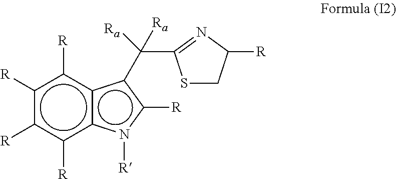

In some preferred embodiments of the present invention, A is

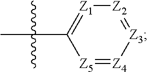

and in which case, formula (I) becomes formula (I3),

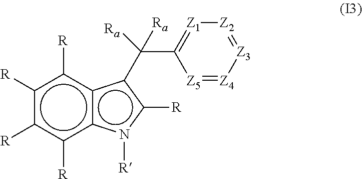

in formula (I3), Z₁ to Z₅ are C(Q), i.e., A being a benzene ring;
or one or two of Z₁ to Z₅ are N, and the others are each independently C(Q); that is,
A may also be a pyridine ring; and in which case, Z₁ is N, and Z₂ to Z₅ are each individually C(Q); or Z₂ is N, and Z₁ and Z₃ to Z₅ are each independently C(Q); or Z₃ is N, and Z₁, Z₂, Z₄, and Z₅ are each independently C(Q);
A may also be a pyridazine ring; and in which case, Z₁ and Z₂ are N, and Z₃ to Z₅ are each individually C(Q); or Z₂ and Z₃ are N, and Z₁, Z₄, and Z₅ are each individually C(Q);
A may also be a pyrimidine ring; and in which case, Z₁ and Z₃ are N, and Z₂, Z₄, and Z₅ are each individually C(Q);
A may also be a piperazine ring; and in which case, Z₁ and Z₄ are N, and Z₂, Z₃, and Z₅ are each individually C(Q);
or adjacent two of Z₁ to Z₅ are C(Q) which together form a 5 to 6 membered carbocyclic ring or a 5 to 6 membered heterocyclic ring interrupted by 1 to 3 heteroatoms selected from N, 0, and S, and the other three are each independently C(Q), or two of the other three are each individually C(Q) and the remaining one is N, or one of the other three is C(Q) and the remaining two are N; the following two situations exist when it is described according to classification of ring formation position: when Z₁ and Z₂ are C(Q) and form a 5 to 6 membered carbocyclic ring or a 5 to 6 membered heterocyclic ring interrupted by 1 to 3 heteroatoms selected from N, 0, and S, Z₃ to Z₅ are each individually C(Q), or Z₃ and Z₄ are each independently C(Q) and Z₅ is N, or Z₃ and Z₅ are each independently C(Q) and Z₄ is N, or Z₄ and Z₅ are each individually C(Q) and Z₃ is N, or Z₃ is C(Q) and Z₄ and Z₅ is N, or Z₄ is C(Q) and Z₃ and Z₅ are N, or Z₅ is C(Q) and Z₃ and Z₄ are N; when Z₂ and Z₃ is C(Q) and form a 5 to 6 membered carbocyclic ring or a 5 to 6 membered heterocyclic ring interrupted by 1 to 3 heteroatoms selected from N, 0, and S, Z₁, Z₄, and Z₅ are each independently C(Q), or Z₁ and Z₄ are each independently C(Q) and Z₅ is N, or Z₁ and Z₅ are each independently C(Q) and Z₄ is N, or Z₄ and Z₅ are each individually C(Q) and Z₁ is N, or Z₁ is C(Q) and Z₄ and Z₅ are N, or Z₄ is C(Q) and Z₁ and Z₅ are N, or Z₅ is C(Q) and Z₁ and Z₄ are N.

In some preferred embodiments of the present invention, R′ is

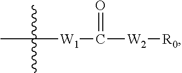

W₁ is a linker bond, C(R₀)₂O, or C(R₀)₂OC(R₀)₂; W₂ is O or CH(N(R₀)₂)R₀.

In specific embodiments, individual functional groups or substituents can be arbitrarily selected and combined within the ranges described; for example, in formula (I), R′ may be one of the following substituents:

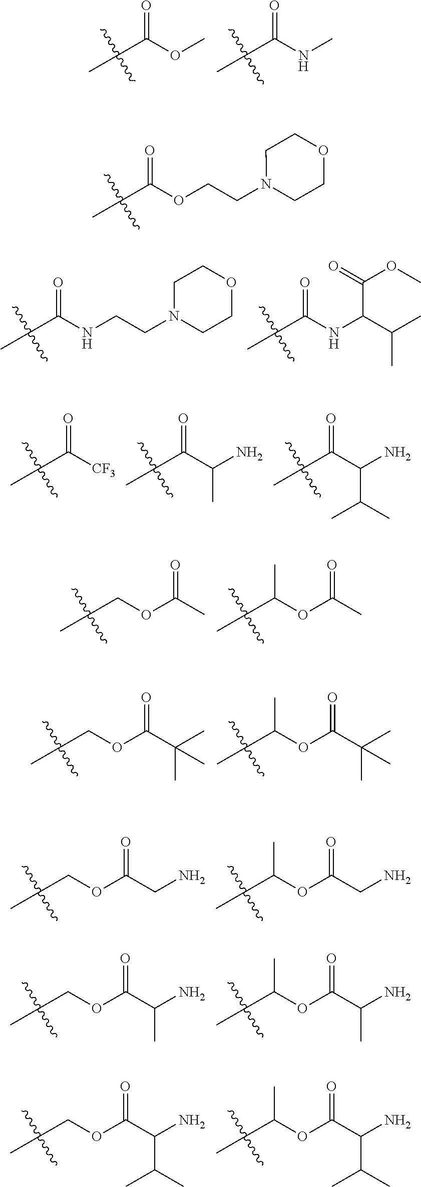

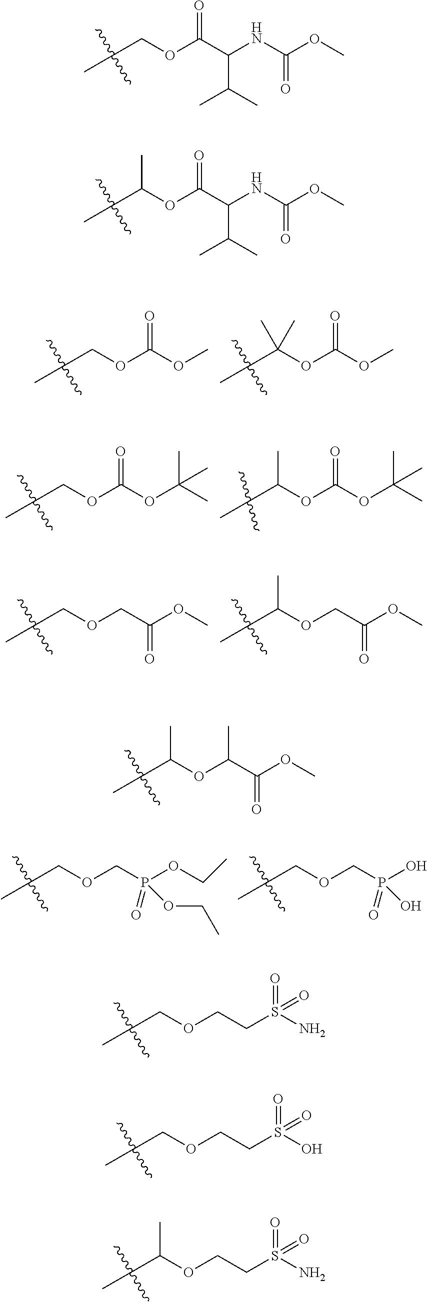

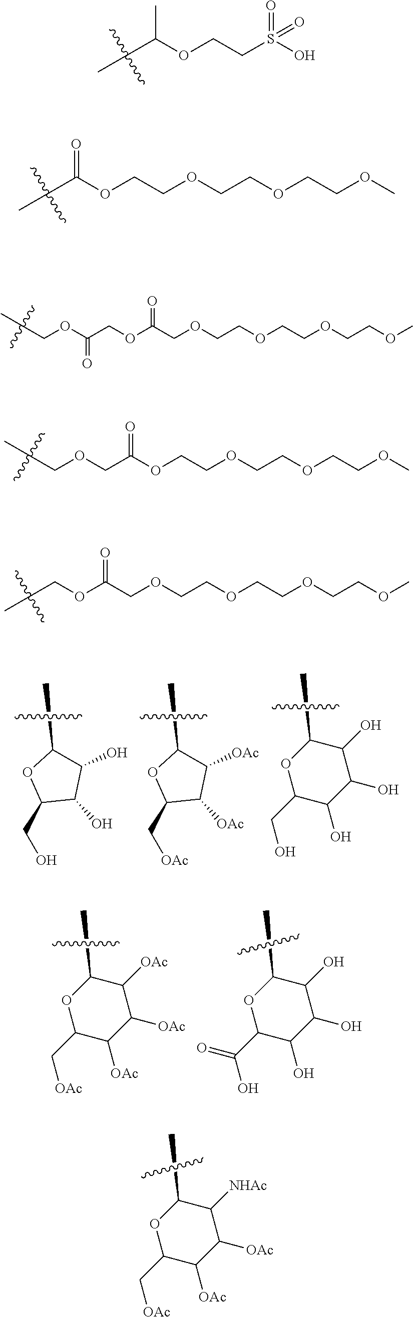

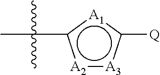

in formula (I1) may be one of the following substituents:

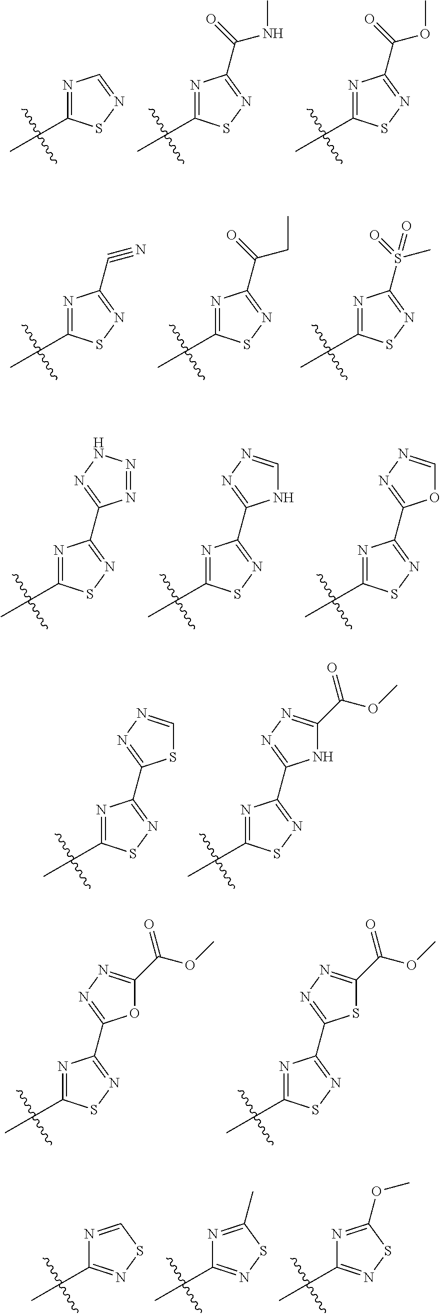

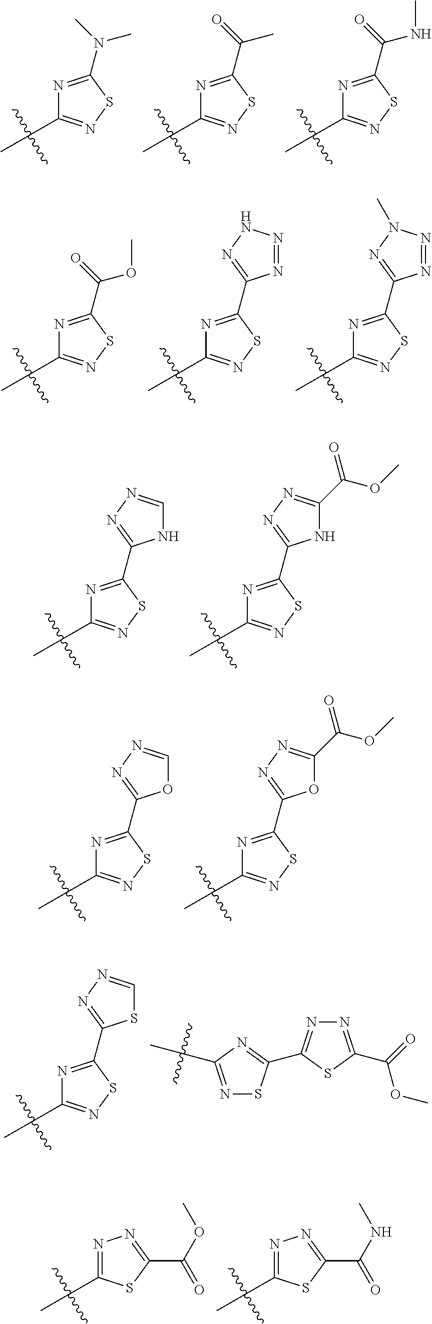

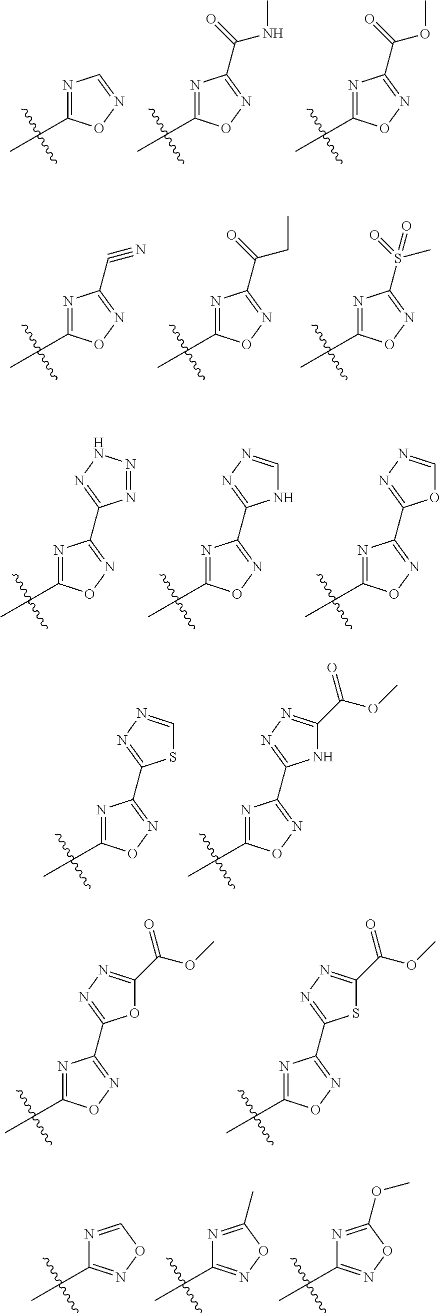

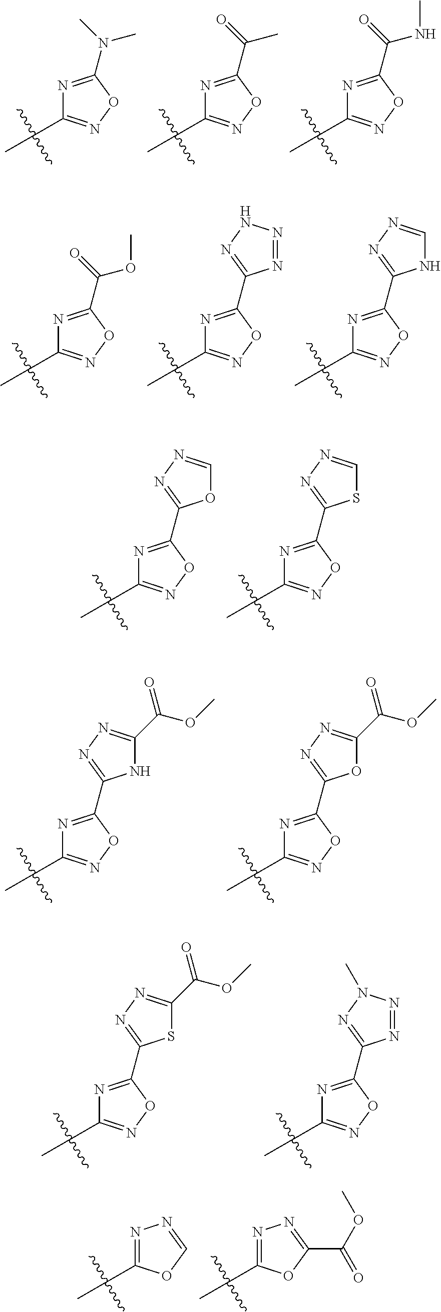

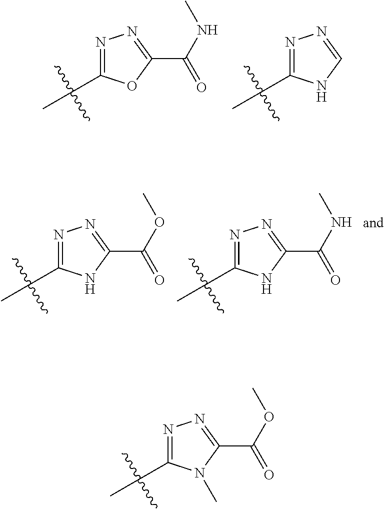

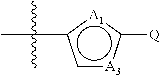

in formula (Ib) may be one of the following substituents:

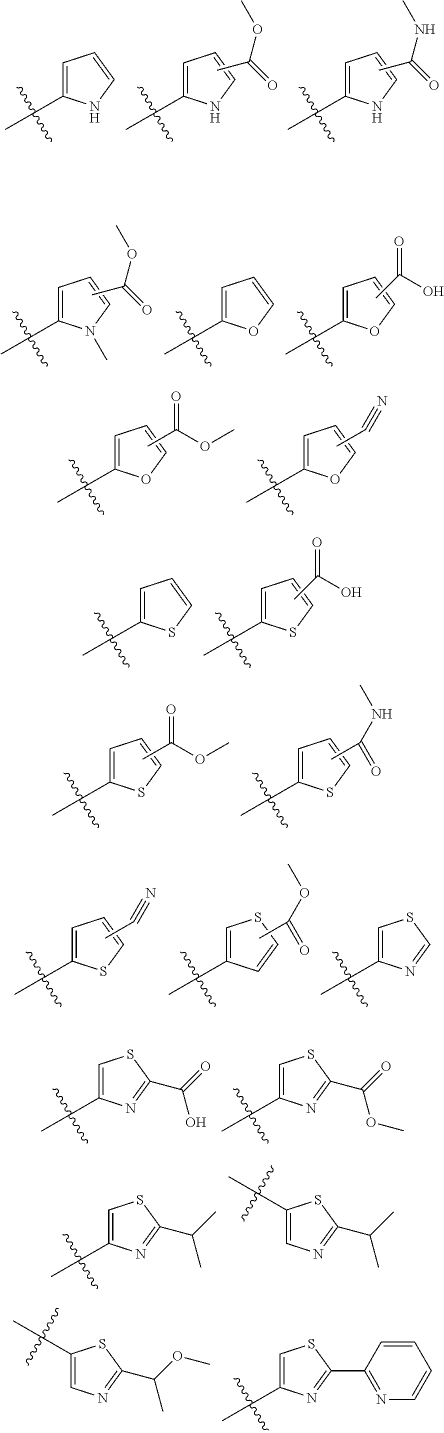

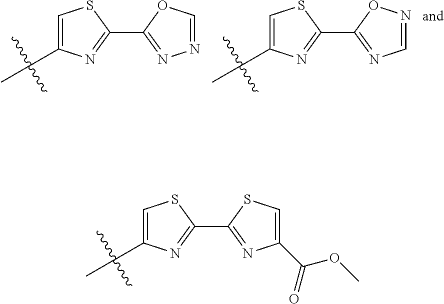 [SMILES: *c1csc(-c2nnco2)n1 ; *c1csc(-c2ncno2)n1 ; "and" ; *c1csc(-c2nc(C(=O)OC)cs2)n1]

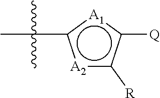

in formulas (Ic) to (If) may be one of the following substituents:

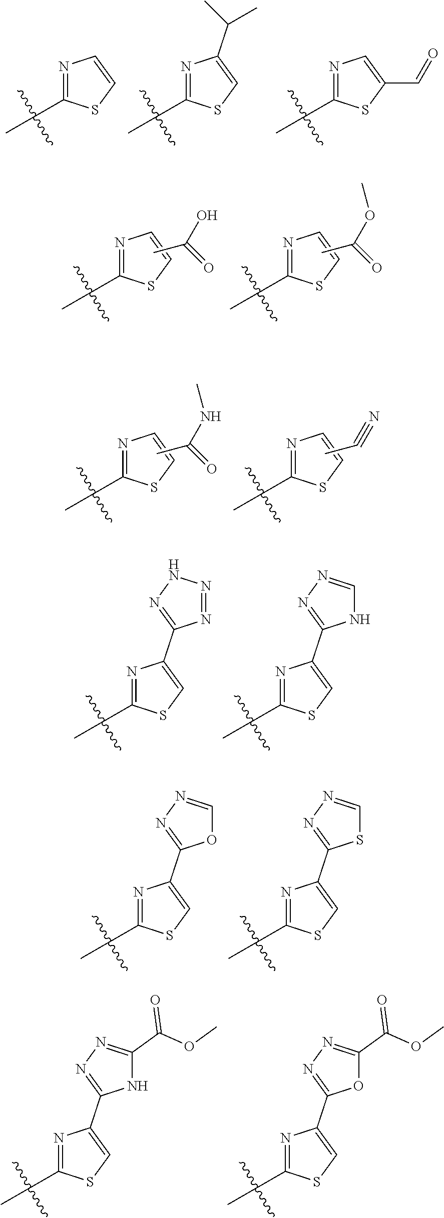

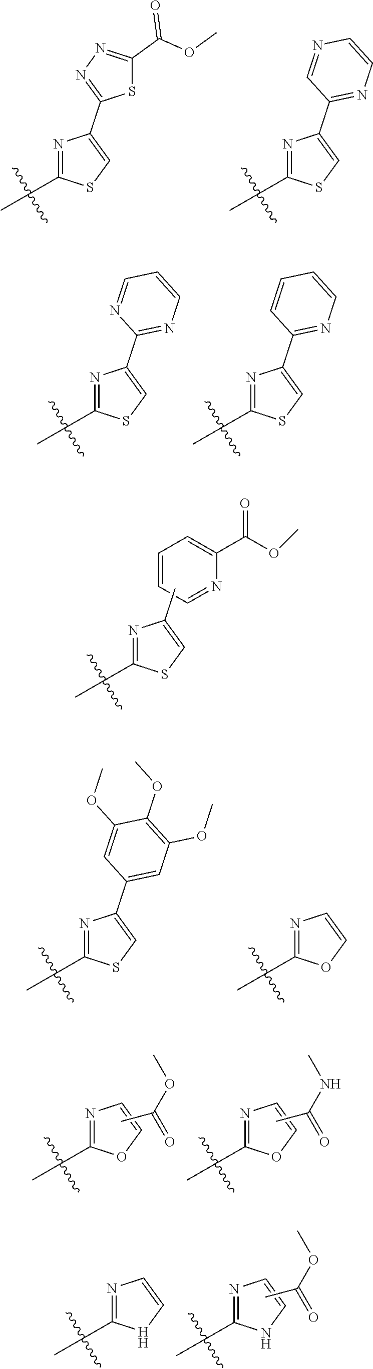

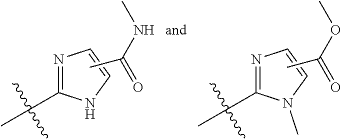

The compounds satisfying formula (Ia) may be

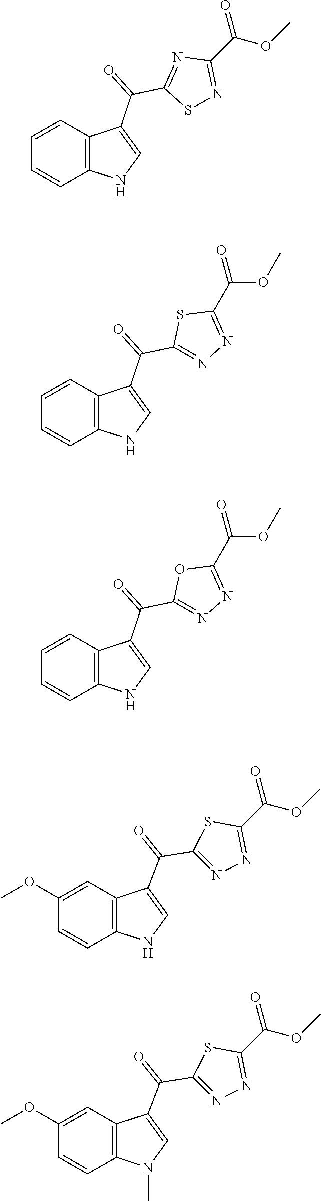

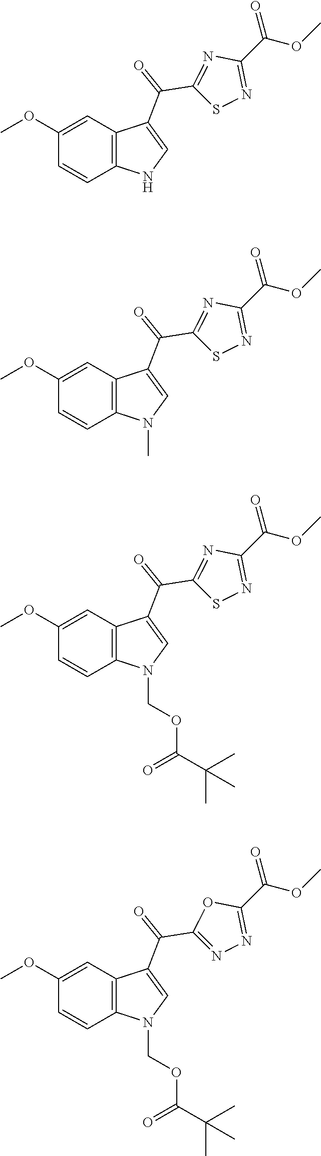

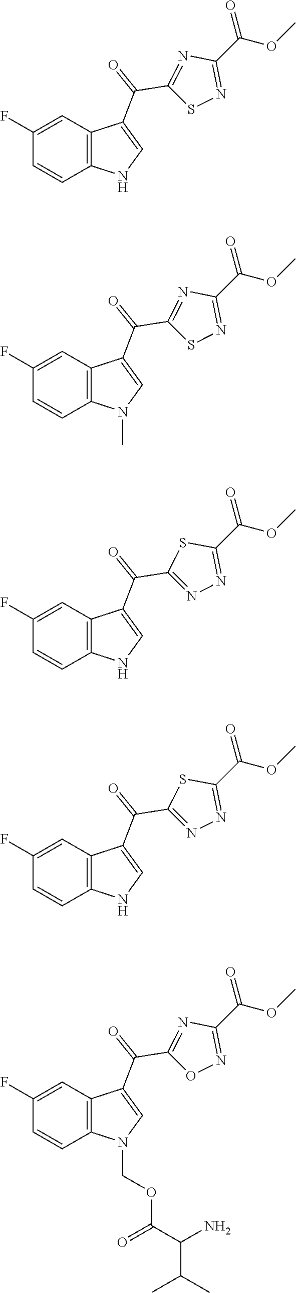 [SMILES: COC(=O)c1nsc(C(=O)c2c[nH]c3ccc(F)cc23)n1 ; COC(=O)c1nsc(C(=O)c2cn(C)c3ccc(F)cc23)n1 ; COC(=O)c1nnc(C(=O)c2c[nH]c3ccc(F)cc23)s1 ; COC(=O)c1nnc(C(=O)c2c[nH]c3ccc(F)cc23)s1 ; COC(=O)c1noc(C(=O)c2cn(COC(=O)C(N)C(C)C)c3ccc(F)cc23)n1]

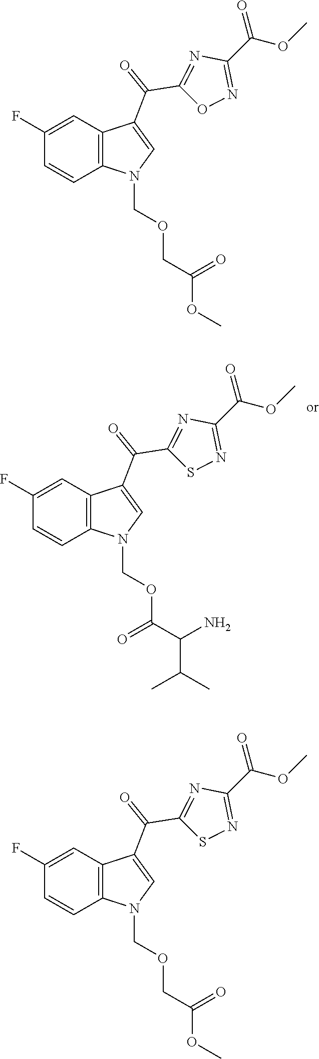

The compounds satisfying formula (Ib) may be

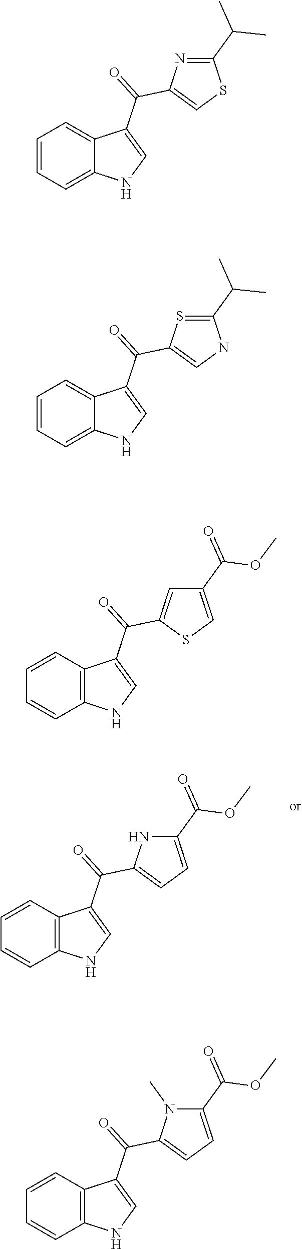

The compounds satisfying formula (Ic) may be

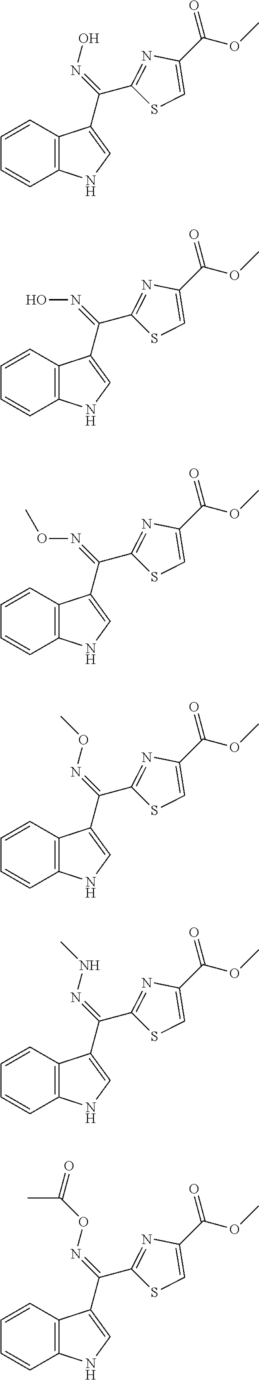 [SMILES: COC(=O)c1csc(/C(=N\O)c2c[nH]c3ccccc23)n1 ; COC(=O)c1csc(/C(=N/O)c2c[nH]c3ccccc23)n1 ; CO/N=C(/c1nc(C(=O)OC)cs1)c1c[nH]c2ccccc12 ; CO/N=C(\c1nc(C(=O)OC)cs1)c1c[nH]c2ccccc12 ; CN/N=C(\c1nc(C(=O)OC)cs1)c1c[nH]c2ccccc12 ; COC(=O)c1csc(/C(=N\OC(C)=O)c2c[nH]c3ccccc23)n1]

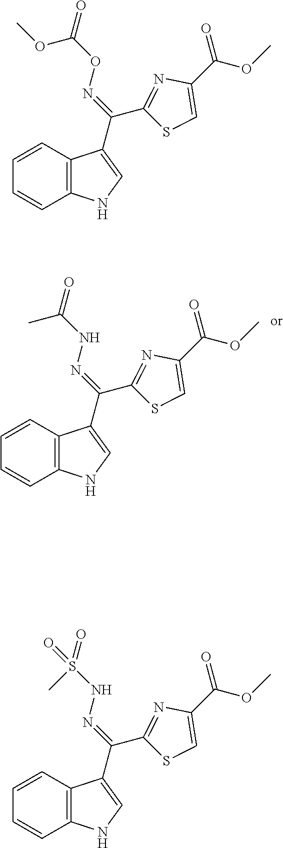 [SMILES: COC(=O)O/N=C(\c1nc(C(=O)OC)cs1)c1c[nH]c2ccccc12 ; COC(=O)c1csc(/C(=N\NC(C)=O)c2c[nH]c3ccccc23)n1 ; "or" ; COC(=O)c1csc(/C(=N\NS(C)(=O)=O)c2c[nH]c3ccccc23)n1]

The compounds satisfying formula (Id) may be

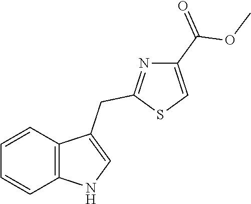 [SMILES: COC(=O)c1csc(Cc2c[nH]c3ccccc23)n1]

The compounds satisfying formula (Ie) may be

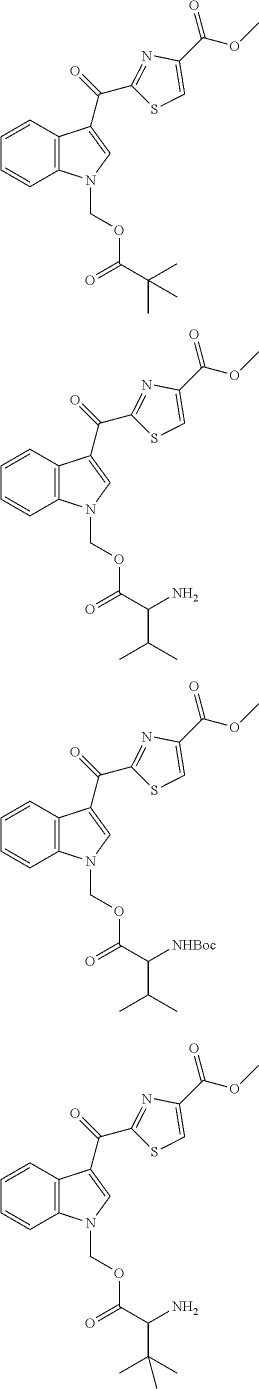

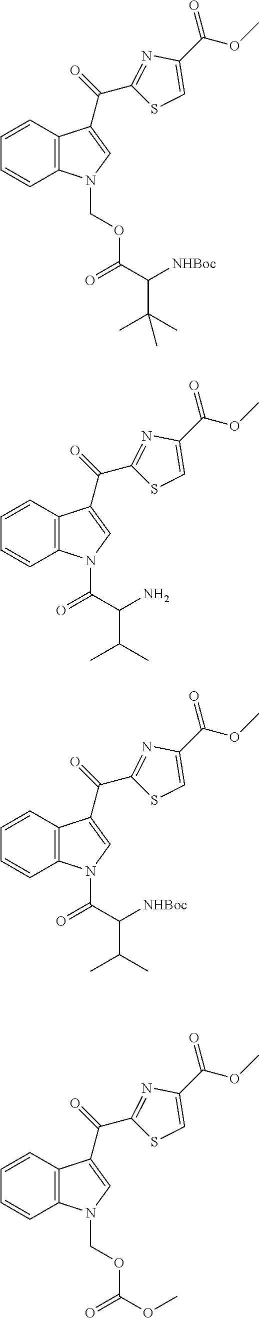 [SMILES: COC(=O)c1csc(C(=O)c2cn(COC(=O)C(NC(=O)OC(C)(C)C)C(C)(C)C)c3ccccc23)n1 ; COC(=O)c1csc(C(=O)c2cn(C(=O)C(N)C(C)C)c3ccccc23)n1 ; COC(=O)c1csc(C(=O)c2cn(C(=O)C(NC(=O)OC(C)(C)C)C(C)C)c3ccccc23)n1 ; COC(=O)OCn1cc(C(=O)c2nc(C(=O)OC)cs2)c2ccccc21]

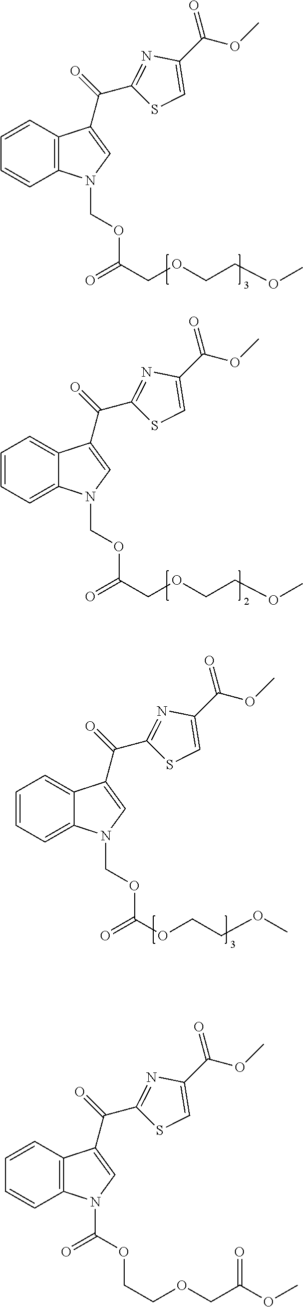

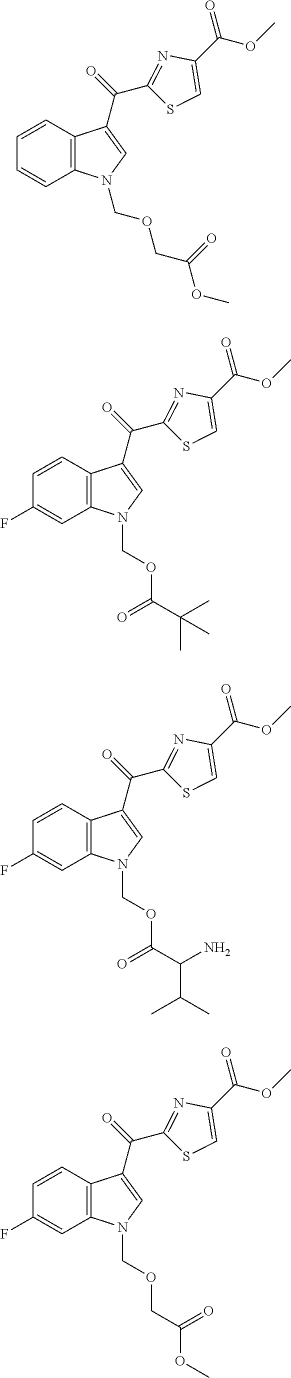

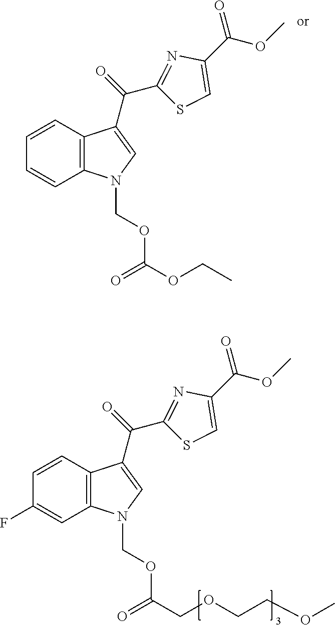

The compounds satisfying formula (If) may be

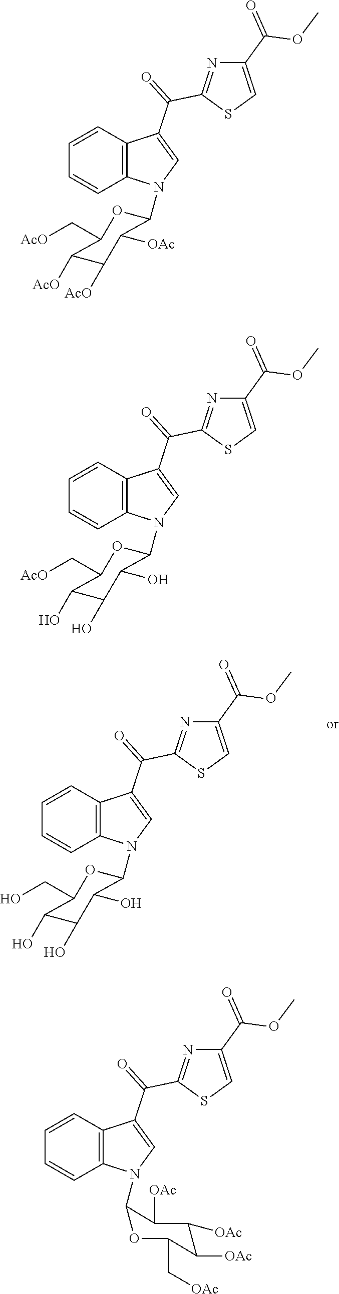 [SMILES: COC(=O)c1csc(C(=O)c2cn(C3OC(COC(C)=O)C(OC(C)=O)C(OC(C)=O)C3OC(C)=O)c3ccccc23)n1 ; COC(=O)c1csc(C(=O)c2cn(C3OC(COC(C)=O)C(O)C(O)C3O)c3ccccc23)n1 ; COC(=O)c1csc(C(=O)c2cn(C3OC(CO)C(O)C(O)C3O)c3ccccc23)n1 ; COC(=O)c1csc(C(=O)c2cn(C3OC(COC(C)=O)C(OC(C)=O)C(OC(C)=O)C3OC(C)=O)c3ccccc23)n1]

The compounds satisfying formula (Ig) may be

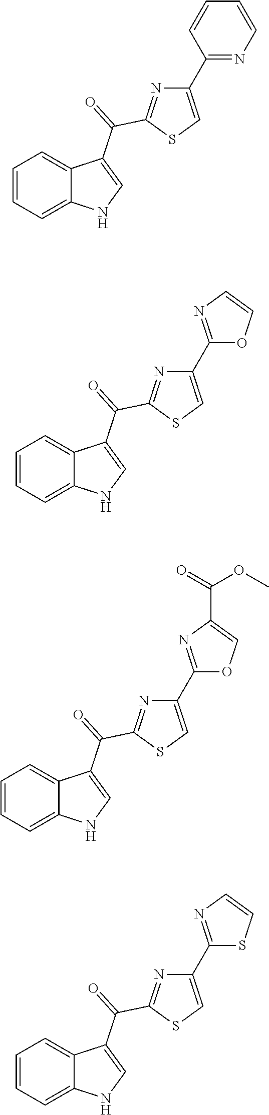 [SMILES: O=C(c1nc(-c2ccccn2)cs1)c1c[nH]c2ccccc12 ; O=C(c1nc(-c2ncco2)cs1)c1c[nH]c2ccccc12 ; COC(=O)c1coc(-c2csc(C(=O)c3c[nH]c4ccccc34)n2)n1 ; O=C(c1nc(-c2nccs2)cs1)c1c[nH]c2ccccc12]

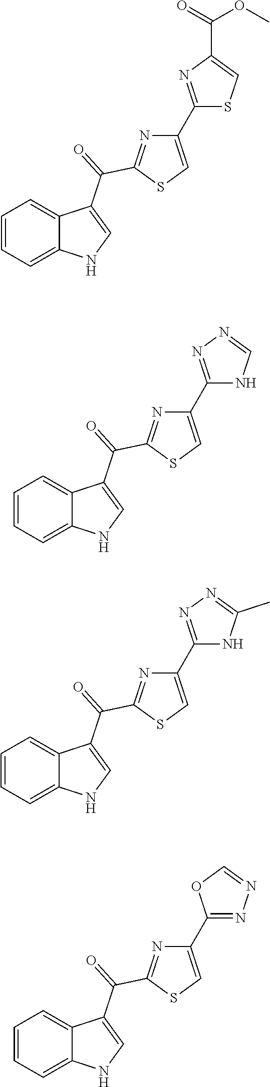 [SMILES: COC(=O)c1csc(-c2csc(C(=O)c3c[nH]c4ccccc34)n2)n1 ; O=C(c1nc(-c2nnc[nH]2)cs1)c1c[nH]c2ccccc12 ; Cc1nnc(-c2csc(C(=O)c3c[nH]c4ccccc34)n2)[nH]1 ; O=C(c1nc(-c2nnco2)cs1)c1c[nH]c2ccccc12]

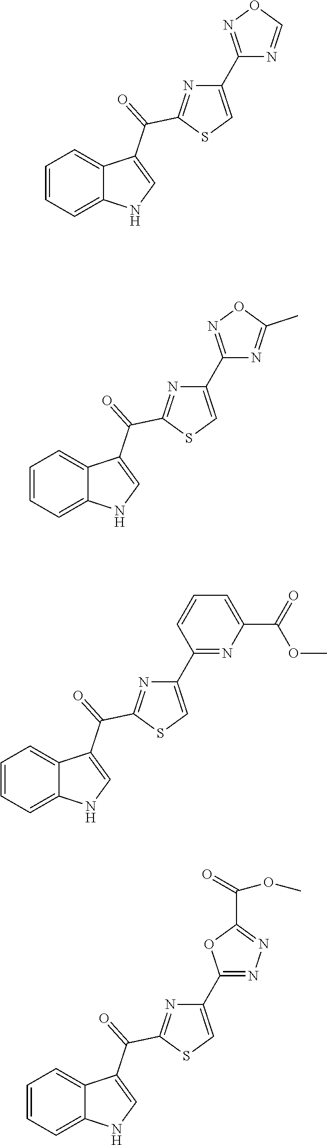 [SMILES: O=C(c1nc(-c2ncon2)cs1)c1c[nH]c2ccccc12 ; Cc1nc(-c2csc(C(=O)c3c[nH]c4ccccc34)n2)no1 ; COC(=O)c1cccc(-c2csc(C(=O)c3c[nH]c4ccccc34)n2)n1 ; COC(=O)c1nnc(-c2csc(C(=O)c3c[nH]c4ccccc34)n2)o1]

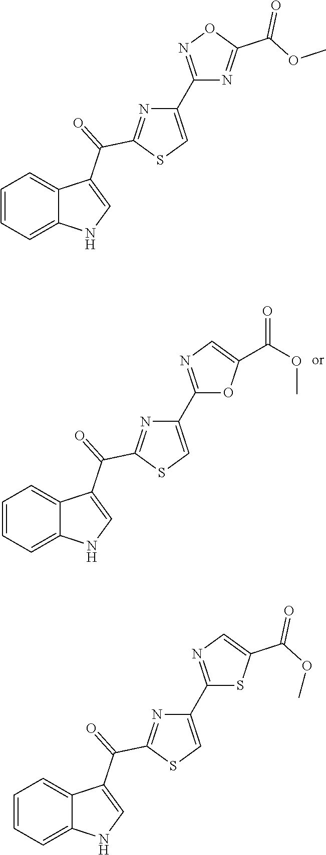 [SMILES: COC(=O)c1nc(-c2csc(C(=O)c3c[nH]c4ccccc34)n2)no1 ; COC(=O)c1cnc(-c2csc(C(=O)c3c[nH]c4ccccc34)n2)o1 ; "or" ; COC(=O)c1cnc(-c2csc(C(=O)c3c[nH]c4ccccc34)n2)s1]

The compounds satisfying formula (Ih) may be

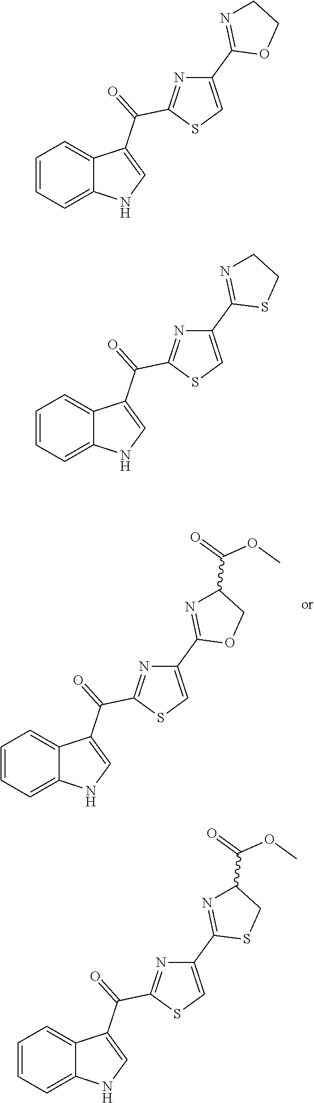 [SMILES: O=C(c1nc(C2=NCCO2)cs1)c1c[nH]c2ccccc12 ; O=C(c1nc(C2=NCCS2)cs1)c1c[nH]c2ccccc12 ; COC(=O)C1COC(c2csc(C(=O)c3c[nH]c4ccccc34)n2)=N1 ; COC(=O)C1CSC(c2csc(C(=O)c3c[nH]c4ccccc34)n2)=N1]

The compounds satisfying formula (II) may also be

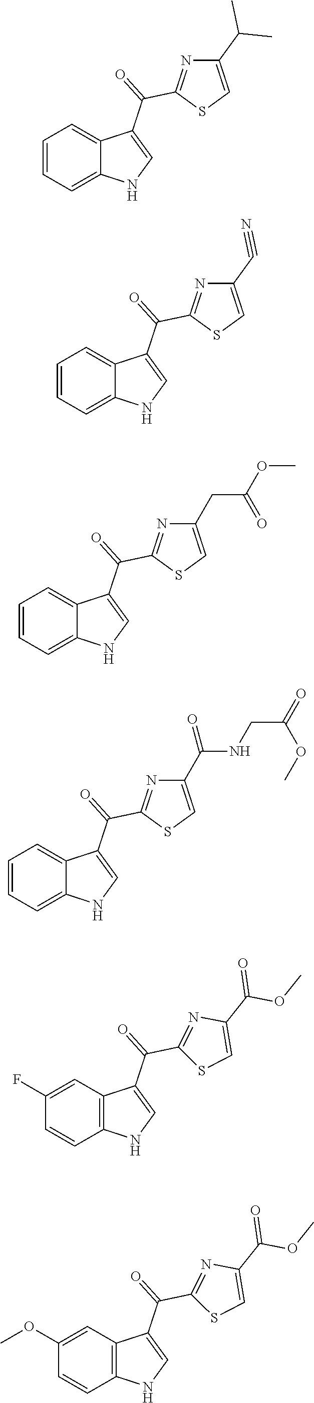

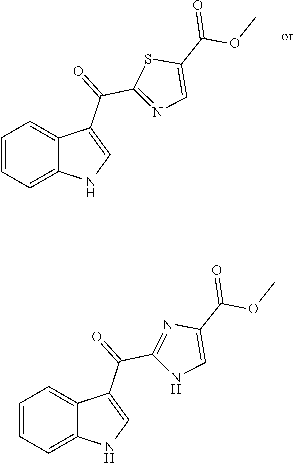 [SMILES: COC(=O)c1cnc(C(=O)c2c[nH]c3ccccc23)s1 ; COC(=O)c1c[nH]c(C(=O)c2c[nH]c3ccccc23)n1]

The compounds satisfying formula (I2) may be

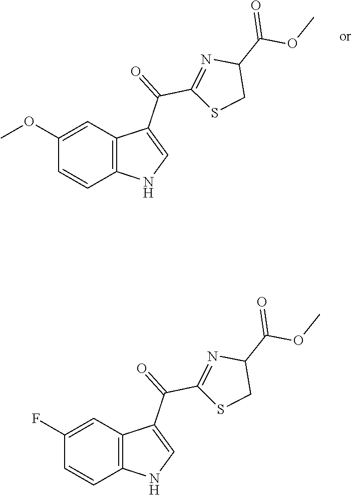

The compounds satisfying formula (I2) may be

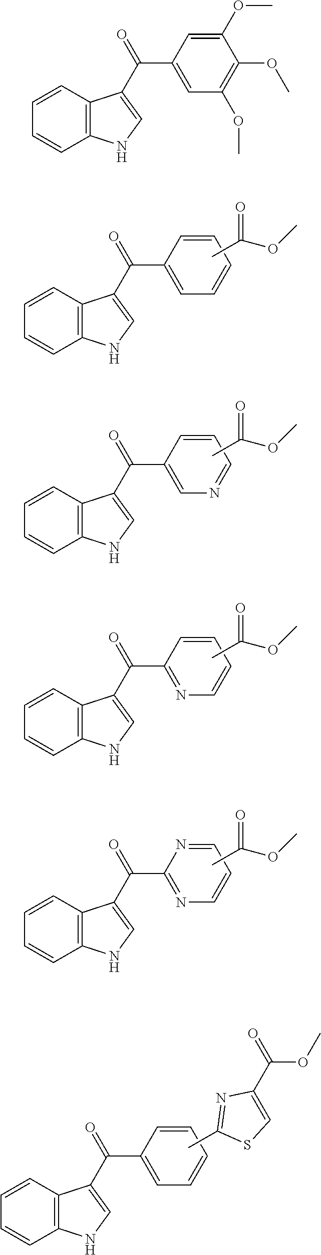

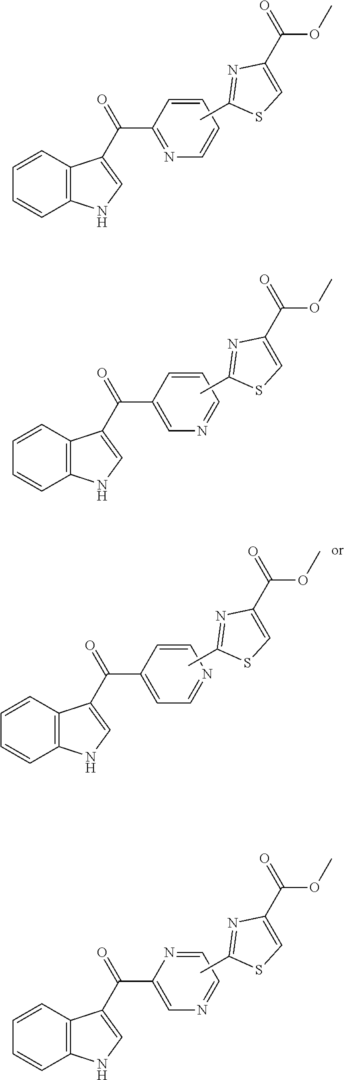

The aryl hydrocarbon receptor modulators of formula (I) of the present invention are classified into the following 5 compounds:

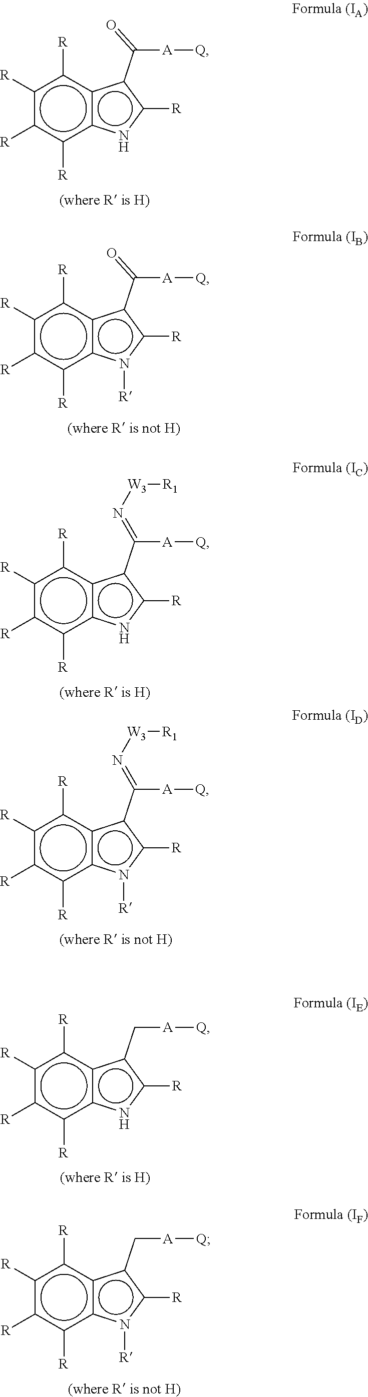

the synthesis scheme of formulas (I_{A}) to formula (I_{F}) is as follows:

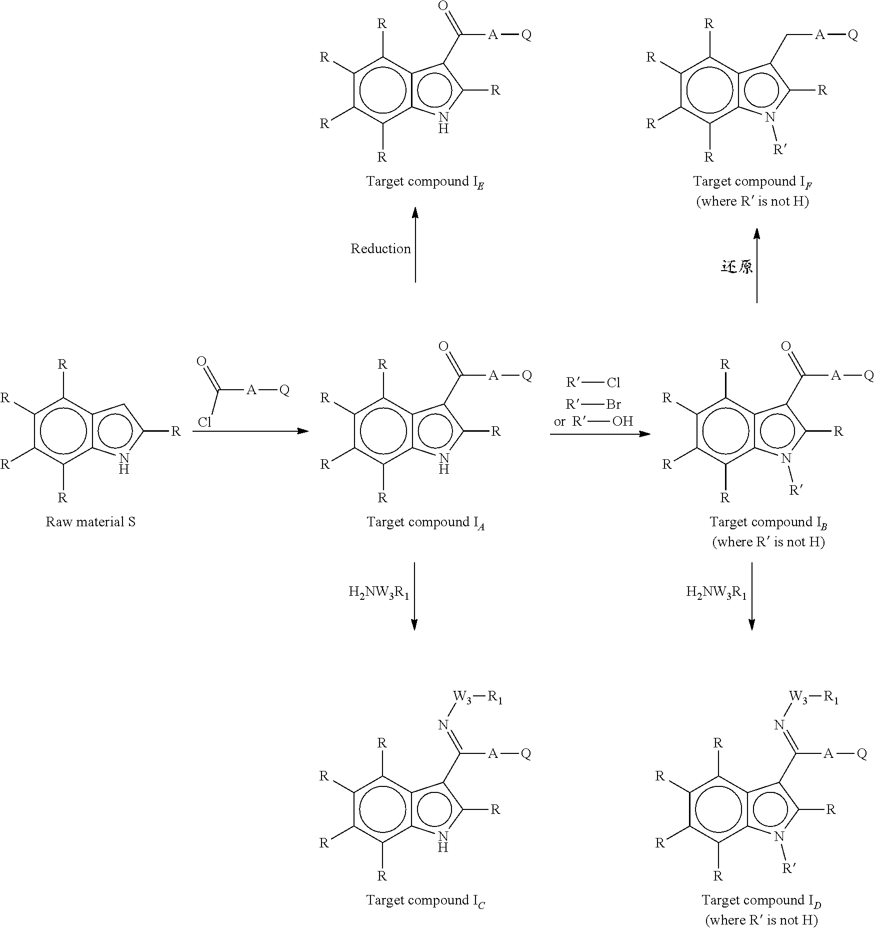

Step 1: Starting material S (indole or an indole derivative) and an acyl halide compound (ClC(O)AQ) and an alcohol or olefinic compound are subjected to a Friedel-Crafts reaction to yield a target compound I_{A} SUBSTITUTED AT POSITION 3 OF INDOLE;
Step 2: The target compound I_{A} is reacted with R′X or R′OH to yield a target compound I_{B};
Step 3: The target compound I_{A} or the target compound I_{B} is reacted with H₂NW₃R₁ to yield a target compound I_{c} or a target compound I_{D}; AND
Step 4: The target compound I_{A} or the target compound I_{B} is subjected to a reduction reaction to yield a target compound I_{E} or a target compounds I_{F}.

Advantageous effect of the present invention: the compounds of formula (I) of the present invention can bind with AhR to regulate those functions and signaling pathways controlled by AhR, thereby affecting the growth and proliferation of cancer cells and the aggressiveness of tumor cells. The pharmaceutical compositions of the compounds of formula (I) can be used as AhR inhibitors or non-constitutive AhR agonists to inhibit the growth of cancer cells and inhibit the metastasis and invasion of tumor cells.

### DETAILED DESCRIPTION

### Embodiment 1. Compound 1-1 and Compound 1-2

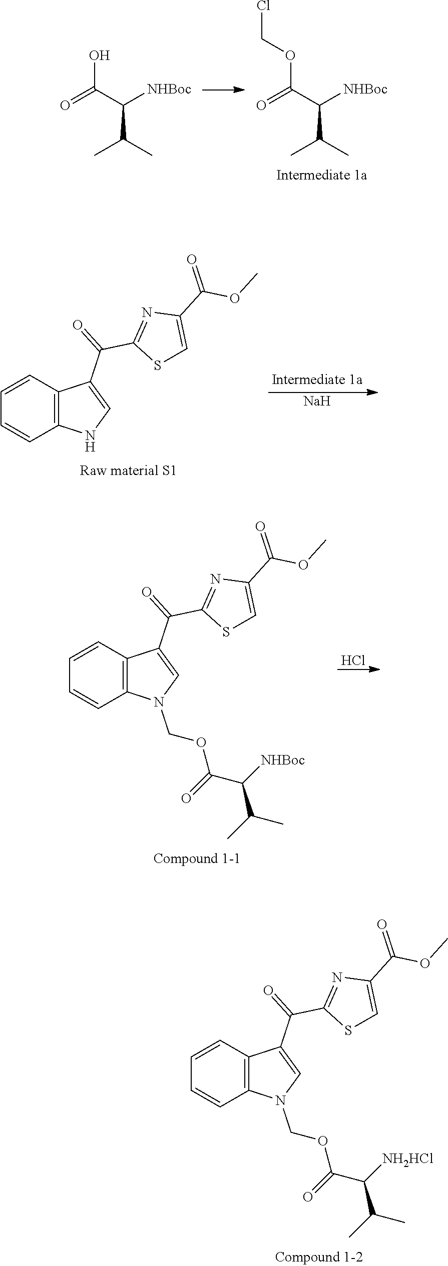

### Intermediate 1a

### Synthesis of Intermediate 1a

Sodium bicarbonate (1.546 g, 18.411 mmol) and tetrabutylammonium bromide (0.237 g, 0.736 mmol) was added into a suspension of Boc-L-valine (0.8 g, 3.68 mmol) in dichloromethane and water (12 mL/12 mL) with stirring; and the reaction was cooled to below 0° C.; and chloromethyl chlorosulphate (0.91 g, 5.52 mmol) was slowly added dropwise to the reaction and then stirred overnight. The reaction was extracted 2 times with dichloromethane; and the organic phase was washed once with water and once with saturated brine, and dried using anhydrous sodium sulfate and concentrated under reduced pressure to yield a crude product. The crude product was purified using silica gel column chromatography (PE/EA=20/1) to yield intermediate 1a as oil (0.97 g, 99% as the yielding rate).

### Synthesis of Compound 1-1

Sodium hydride (0.165 g, 4.139 mmol), in batch, was added to a solution of raw material S1 (1 g, 3.763 mmol) of dimethylformamide (DMF) (10 mL) with stirring; the temperature was raised to 40° C. for reaction for 1 h and reduced to room temperature; and a solution of intermediate 1a (0.97 g, 3.6 mmol) in DMF (2 mL) was slowly added dropwise therein; and the mixture was stirred overnight at room temperature. The reaction was poured into ice water of 60 mL and filtered to yield a crude product. The crude product was purified using silica gel column chromatography (PE/EA=20/1 to 10/1) to yield compound 1-1 (yield: 0.5 g, 28%). MS(ESI) m/z: 516 [M+1]⁺.

### Synthesis of Compound 1-2

Compound 1-1 (0.5 g, 0.97 mmol) was dissolved in dioxane (2 mL); dropwise hydrogen chloride/dioxane (5 mL) was added therein; and the mixture was reacted at room temperature overnight to yield compound 1-2 (yield: 0.24 g, 55%). ¹H NMR (400 MHz, CDCl₃): δ 9.24 (s, 1H), 8.94 (s, 1H), 8.41 (brs, 3H), 8.35 (d, J=7.6 Hz, 1H), 7.81 (d, J=7.6 Hz, 1H), 7.39 to 7.47 (m, 2H), 6.63 (d, J=10.8 Hz, 1H), 6.58 (d, J=10.8 Hz, 1H), 4.02 (d, J=7.6 Hz, 1H), 3.94 (s, 3H), 2.07-2.12 (m, 1H), 0.84 (d, J=7.2 Hz, 1H), 0.80 (d, J=7.2 Hz, 1H). MS(ESI) m/z: 416 [M+1].

### Embodiment 2. Compound 2-1 and Compound 2-2

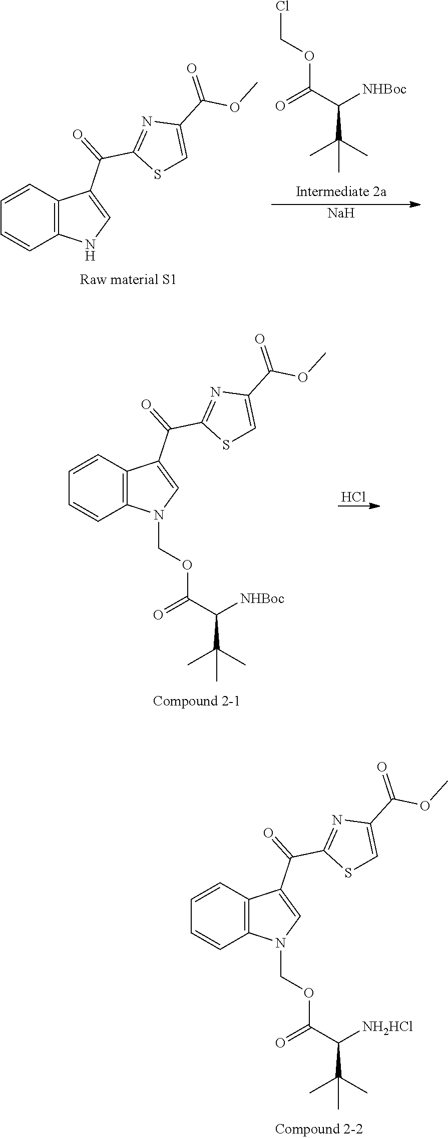

### Synthesis of Intermediate 2a

The method was the same as the synthesis method for intermediate 1a; Boc-L-tert-leucine (2 g, 8.647 mmol) was used to prepare intermediate 2a as oil (yield: 2.3 g, 95%).

### Synthesis of Compound 2-1

The method was the same as the synthesis method for compound 1-1; intermediate 2a (1 g, 3.6 mmol) was used to prepare compound 2-1 (yield: 1.4 g, 74%).

¹H NMR (400 MHz, CDCl₃): δ 9.24 (s, 1H), 8.50-8.52 (m, 1H), 8.46 (s, 1H), 7.57 to 7.60 (m, 1H), 7.39-7.43 (m, 1H), 6.42 (d, J=11.2 Hz, 1H), 6.17 (d, J=11.2 Hz, 1H), 5.05 (d, J=9.2 Hz, 1H), 4.10 (d, J=8.4 Hz, 1H), 4.04 (s, 3H), 1.42 (s, 9H), 0.83 (s, 9H). MS(ESI) m/z: 530 [M+1]⁺.

### Synthesis of Compound 2-2

The method was the same as the synthesis method for compound 1-2; compound 2-1 (1.4 g, 2.6 mmol) was used to prepare compound 2-2 (yield: 0.85 g, 70%). ¹H NMR (400 MHz, CDCl₃): δ 9.24 (s, 1H), 8.94 (s, 1H), 8.36 (d, J=7.2 Hz, 1H), 8.27 (brs, 3H), 7.82 (d, J=7.6 Hz, 1H), 7.39 to 7.47 (m, 2H), 6.61 (s, 1H), 3.93 (s, 3H), 3.86 (s, 3H), 0.89 (s, 9H). MS(ESI) m/z: 430 [M+1]⁺.

### Embodiment 3. Compound 3

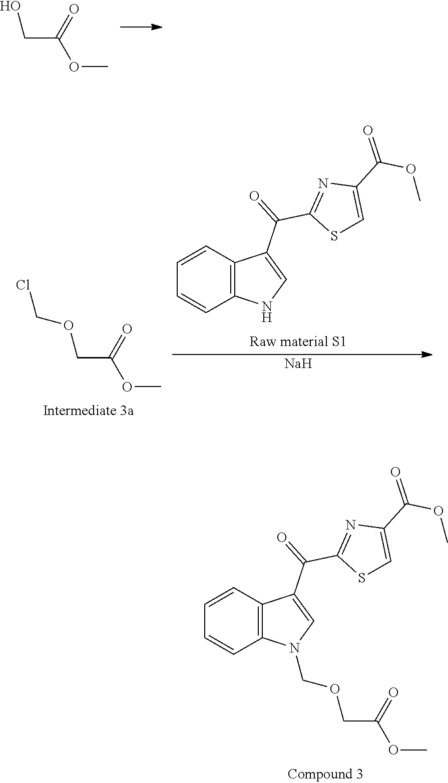

### Embodiment 3

### Synthesis of Intermediate 3a

Methyl hydroxyacetate (3 g, 33.3 mmol) was weighed; dichloromethane (50 mL) and paraformaldehyde (1.3 g, 43.3 mmol) were added therein; the temperature was reduced to below −20° C., and freshly made hydrogen chloride gas was continuously charged thereinto. The reaction was kept at −20° C. for 30 min. Hydrogen chloride gas was removed, anhydrous magnesium sulfate and anhydrous sodium sulfate were added therein; and the temperature of the reaction was kept for another hour; and the reaction was kept overnight at room temperature. The solid was removed by filtration and the mother liquor was concentrated to dryness at room temperature and then purified using silica gel column chromatography to yield intermediate 3a (yield: 1.2 g, 26%).

### Synthesis of Compound 3

The method was the same as the synthesis method for compound 1-1; raw material S1 (286 mg, 1 mmol) and intermediate 3a (500 mg, 3.6 mmol) were used to prepare compound 3 as light yellow solid (yield: 280 mg, 74%). ¹H NMR (400 MHz, CDCl₃): δ 9.19 (s, 1H), 8.55-8.56 (m, 1H), 8.45 (s, 1H), 7.63-7.65 (m, 1H), 7.41-7.45 (m, 2H), 5.82 (s, 2H), 4.12 (s, 2H), 4.03 (s, 3H), 3.77 (s, 3H). MS (ESI) m/z: 389 [M+1]⁺.

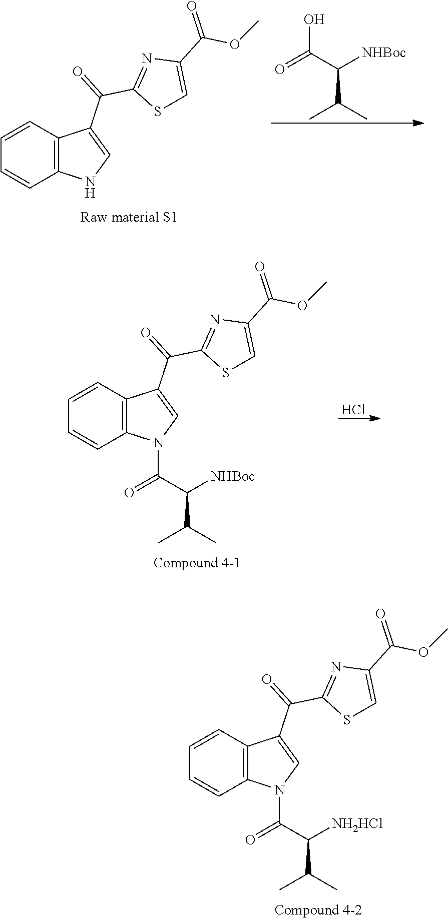

### Synthesis of Compound 4-1

To Raw material S1 (2.86 g, 10 mmol) was added to a solution of Boc-L-valine (2.17 g, 10 mmol) in DMF (20 mL); and HATU (4.56 g, 12 mmol) and DIEA (2.6 g, 20 mmol) were added therein with stirring. The mixture was stirred overnight. The reaction was poured into water and extracted 2 times with ethyl acetate. The organic phase was washed once with water and once with saturated brine, dried using anhydrous sodium sulfate and concentrated under reduced pressure to yield a crude product. The crude product was purified using silica gel column chromatography (PE/EA=4/1) to yield compound 4-1 (yield: 3.01 g, 62%). ¹HNMR (400 MHz, CDCl₃): δ 9.75 (s, 1H), 8.48-8.55 (m, 3H), 7.47-7.52 (m, 2H), 5.44 (d, J=8.8 Hz, 1H), 5.27 (dd, J=4.0, 8.8 Hz, 1H), 4.05 (s, 3H), 2.37-2.42 (m, 1H), 1.48 (s, 9H), 1.25 (d, J=6.8 Hz, 3H), 1.01 (d, J=6.4 Hz, 3H). MS (ESI) m/z: 508 [M+23].

### Synthesis of Compound 4-2

The method was the same as the synthesis method for compound 1-2; compound 4-1 (486 mg, 1 mmol) was used to prepare compound 4-2 (yield: 348 mg, 77%). H NMR (400 MHz, CDCl₃): δ 9.56 (s, 1H), 9.04 (s, 1H), 8.81 (brs, 3H), 8.46-8.48 (m, 1H), 8.35-8.37 (s, 1H), 7.54 to 7.60 (m, 2H), 5.01 (d, J=4.8 Hz, 1H), 3.99 (s, 3H), 2.42-2.47 (m, 1H), 1.17 (d, J=6.8 Hz, 3H), 1.07 (d, J=6.8 Hz, 3H). MS (ESI) m/z: 386 [M+1]⁺.

### Embodiment 5. Compound 5

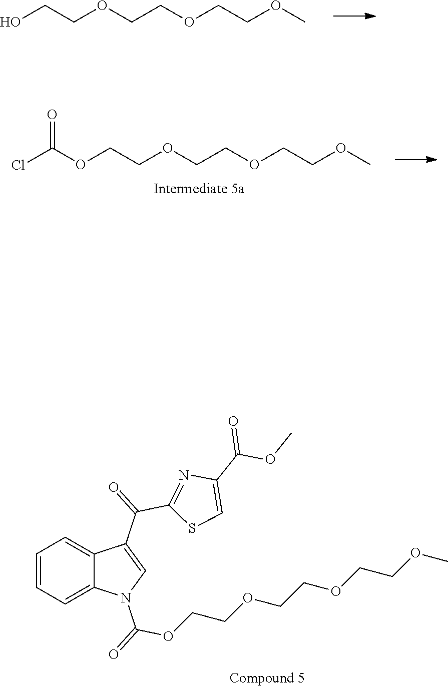 [SMILES: COCCOCCOCCO ; COCCOCCOCCOC(=O)Cl ; COCCOCCOCCOC(=O)n1cc(C(=O)c2nc(C(=O)OC)cs2)c2ccccc21]

### Synthesis of Intermediate 5a

Triethylene glycol monomethyl ether (2.0 g, 12.2 mmol) was dissolved in tetrahydrofuran (20 mL); triphosgene (1.8 g, 6.1 mmol) was added therein with stirring; the temperature was reduced to zero with an ice bath; and pyridine (1.5 g, 19.0 mmol) was slowly added dropwise therein. The mixture was reacted at room temperature for 1 h. The reaction was filtered, and the mother liquor was concentrated under reduced pressure to yield intermediate 5a as colorless liquid (yield: 2.1 g, 75.9%).

### Synthesis of Compound 5

Raw material S1 (2.0 g, 7.0 mmol) was dissolved in tetrahydrofuran (80 ml); triethylamine (1.5 g, 14.9 mmol) was added dropwise therein; the temperature was reduced to zero with an ice bath; and a solution of intermediate 5-1 (2.1 g, 9.3 mmol) in dichloromethane (20 ml) was added dropwise therein. The mixture was reacted at room temperature for 1 h. The reaction was poured into ice water and extracted with dichloromethane. The organic phase was washed with saturated brine, dried using anhydrous sodium sulfate, and concentrated to dryness under reduced pressure. The resulting crude product was purified using silica gel column chromatography (PE/EA=3/1) to yield compound 5 as white solid (yield: 2.5 g, 75.8%). ¹H NMR (400 MHz, CDCl₃): δ 9.56 (s, 1H), 8.49 (s, 2H), 8.33-8.24 (m, 1H), 7.51-7.39 (m, 2H), 4.75-4.67 (m, 2H), 4.03 (s, 3H), 4.01-3.94 (m, 2H), 3.80 (dd, J=5.9, 3.4 Hz, 2H), 3.74-3.69 (m, 2H), 3.67-3.62 (m, 2H), 3.53-3.48 (m, 2H), 3.35 (s, 3H). LCMS(ESI) m/z: 477.2 [M+1]+.

### Embodiment 6. Compound 6

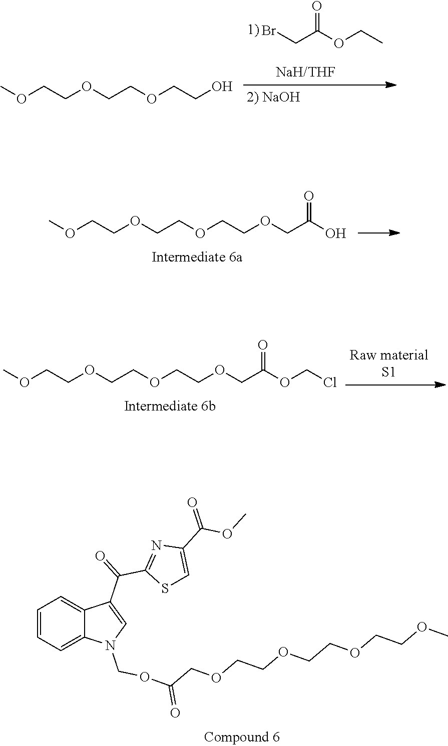

### Synthesis of Intermediate 6a

Triethylene glycol monomethyl ether (10 g, 60.9 mmol) was dissolved in tetrahydrofuran (100 mL); and sodium hydride (3.2 g, 60%, 79.17 mmol) in batches was added at 0° C. Afterwards, the mixture was stirred at room temperature for 1 h. Ethyl bromoacetate (20.1 g, 122 mmol) was added dropwise therein; the mixture was reacted at room temperature for 3 h; and water (100 mL) was directly added to the reaction. The reaction was extracted with dichloromethane, and the organic phase was dried using anhydrous sodium sulfate and concentrated to dryness under reduced pressure. Then, water (100 mL) and solid sodium hydroxide (3 g, 73 mmol) were added therein. The mixture was stirred at room temperature for 1 h and extracted 2 times with ethyl acetate. The aqueous phase was adjusted to pH=2 to 3 with diluted HCl and then extracted 5 times with a mixed solvent of dichloromethane/isopropanol (V/V=10:1); and the organic phase was dried using anhydrous sodium sulfate and concentrated to dryness under reduced pressure to yield a crude product. The crude product was purified using silica gel column chromatography (DCM:CH₃OH=100:1 to 20:1) to yield compound 6a (yield: 10 g, 74%).

### Synthesis of Intermediate 6b

Compound 6a (2 g, 8.99 mmol) was dissolved in dichloromethane (20 mL); and sodium bicarbonate (3.1 g, 36 mmol), tetrabutylammonium bromide (289 mg, 0.899 mmol), and water (20 mL) were added therein. The temperature was reduced to below 0° C.; a solution of chloromethyl chlorosulfonate (1.48 g, 8.99 mmol) in dichloromethane (10 mL) was added dropwise therein; and the mixture was stirred at room temperature overnight. The reaction was allowed to stand until the layers were separated. The aqueous phase was 2 times extracted with dichloromethane, and the organic phase was dried using anhydrous sodium sulfate and concentrated to dryness under reduced pressure to yield a crude product. The crude product was purified using silica gel column chromatography (DCM:CH₃OH=50:1) to yield compound 6b as oily liquid (yield: 300 mg, 12.3%). LCMS (ESI) m/z: 271 [M+1]⁺.

### Synthesis of Compound 6

Raw material S1 (1 g, 3.49 mmol) was dissolved in DMF (15 mL); and sodium hydride (153 mg, 60%, 3.84 mmol) was added therein at 0° C. After the addition, the mixture was stirred for 10 min; and then stirred for 1 h after the temperature was raised to 50° C., and then cooled to at room temperature. Compound 6b (0.944 mg, 3.49 mmol) was added therein and the mixture was reacted at room temperature for 4 h. Water and dichloromethane were added, and the reaction was 3 times extracted with dichloromethane. The organic phase was dried using anhydrous sodium sulfate and concentrated to dryness under reduced pressure to yield a crude product. The crude product was purified using silica gel column chromatography (CH₃OH: DCM=0-2%) to yield compound 6 (yield: 650 mg, 35.8%). ¹H NMR (400 MHz, CDCl₃): δ 9.25 (s, 1H), 8.52-8.54 (m, 1H), 8.46 (s, 1H), 7.59 to 7.61 (m, 1H), 7.41-7.44 (m, 2H), 6.32 (s, 2H), 4.21 (s, 2H), 4.04 (s, 3H), 3.70 to 3.72 (m, 2H), 3.65 to 3.68 (m, 2H), 3.60 to 3.64 (m, 6H), 3.52-3.54 (m, 2H), 3.37 (s, 3H). LCMS (ESI) m/z: 521 [M+1]⁺.

### Embodiment 7. Compound 7

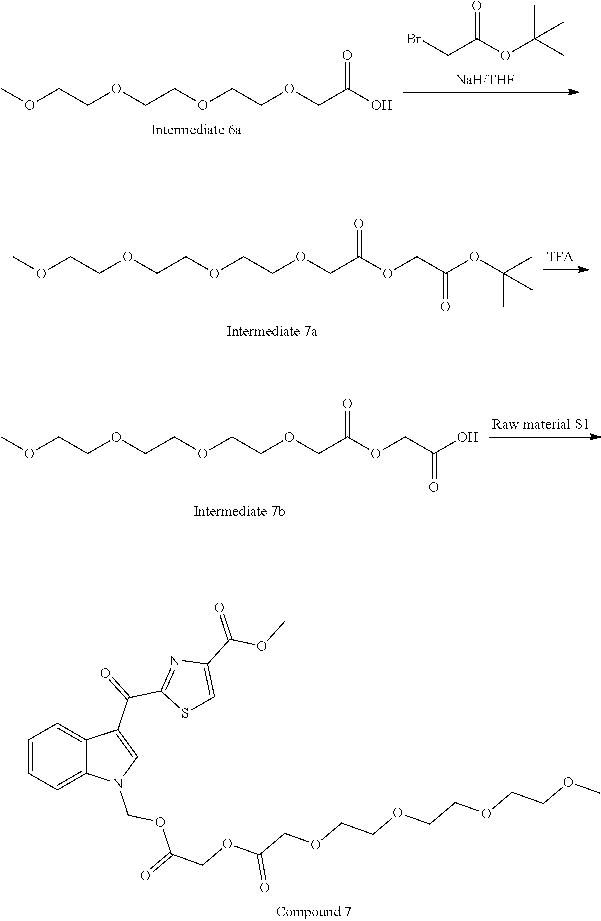

### Synthesis of Intermediate 7a

The method was the same as that for 6a; and the starting material was intermediate 6a. yield: 75%. LCMS (ESI) m/z: 337.2 [M+1]⁺.

### Synthesis of Intermediate 7b

Intermediate 7a (3.4 g, 10 mmol) was dissolved in dichloromethane (5 mL); and trifluoroacetic acid (5 mL) was added therein. The mixture was stirred at room temperature overnight. The reaction was concentrated to dryness under reduced pressure. The resulting crude product was purified using silica gel column chromatography (CH₃OH: DCM=0-2%) to yield compound 7b as oil (yield: 2.6 g, 76%). LCMS (ESI) m/z−. 281.2 [M+1]⁺.

### Compound 7

The method was the same as that for compound 6. yield: 55%. ¹HNMR (400 MHz, CDCl₃): δ 9.20 (s, 1H), 8.50-8.52 (m, 1H), 8.44 (s, 1H), 7.53 to 7.56 (m, 1H), 7.40-7.42 (m, 2H), 6.31 (s, 2H), 4.70 (s, 2), 4.25 (s, 2H), 4.02 (s, 3H), 3.63 to 3.71 (m, 10H), 3.53-3.55 (m, 2H), 3.37 (s, 3H). LCMS (ESI) m/z: 579.2 [M+1]⁺.

### Embodiment 8. Compound 8

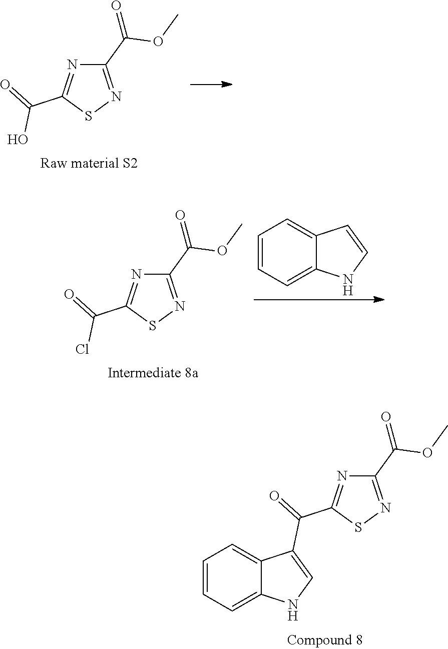

### Synthesis of Intermediate 8a

Raw material S2 (188 mg, 1 mmol) was dissolved in dichloromethane (20 mL), and 1 drop of DMF was added dropwise therein. The mixture was cooled to 0-5° C.; oxalyl chloride (151 mg, 1.2 mmol) was added dropwise therein; ice bath was removed, and the mixture was stirred at room temperature for 1 h. The reaction was concentrated to dryness under reduced pressure. The solid was dissolved with dichloroethane (20 mL), and the solution was concentrated to dryness under reduced pressure to yield intermediate 8a, which was directly used for the next step.

### Synthesis of Compound 8

A solution of intermediate 8a (1 mmol) in dichloromethane (30 mL) was added dropwise to a suspension of anhydrous trialuminum chloride (164 mg, 1.2 mmol) in dichloromethane (30 mL); and the mixture was stirred for 2 h. A solution of indole (143 mg, 1.2 mmol) in dichloromethane (30 mL) was slowly added dropwise to the above reaction; and the mixture was reacted overnight.

The reaction was washed with saturated sodium bicarbonate solution. The organic phase was washed with saturated brine, dried using anhydrous sodium sulfate and concentrated under reduced pressure to yield a crude product. The crude product was purified using silica gel column chromatography (PE/EA=4/1) to yield compound 8 as light yellow solid (yield: 120 mg, 42%). ¹H NMR (400 MHz, DMSO-d₆): δ 12.4 (brs, 1H), 9.05 (s, 1H), 8.28 to 8.30 (m, 1H), 7.62 to 7.64 (m, 1H), 7.32-7.37 (m, 2H), 4.00 (s, 3H). MS (ESI) m/z: 288.0 [M+1]⁺.

### Embodiments 9 to 18. Compounds 9 to 18

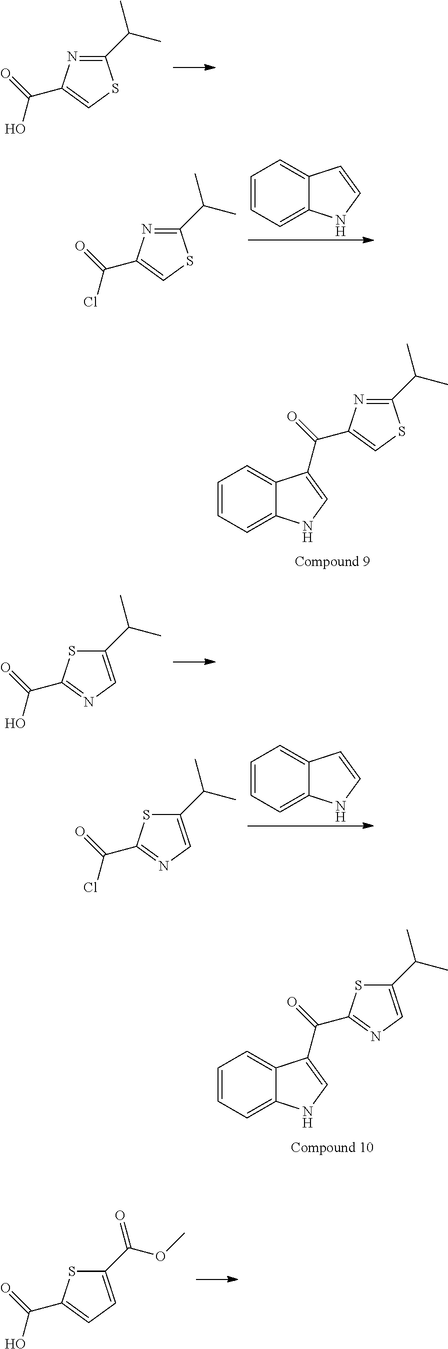

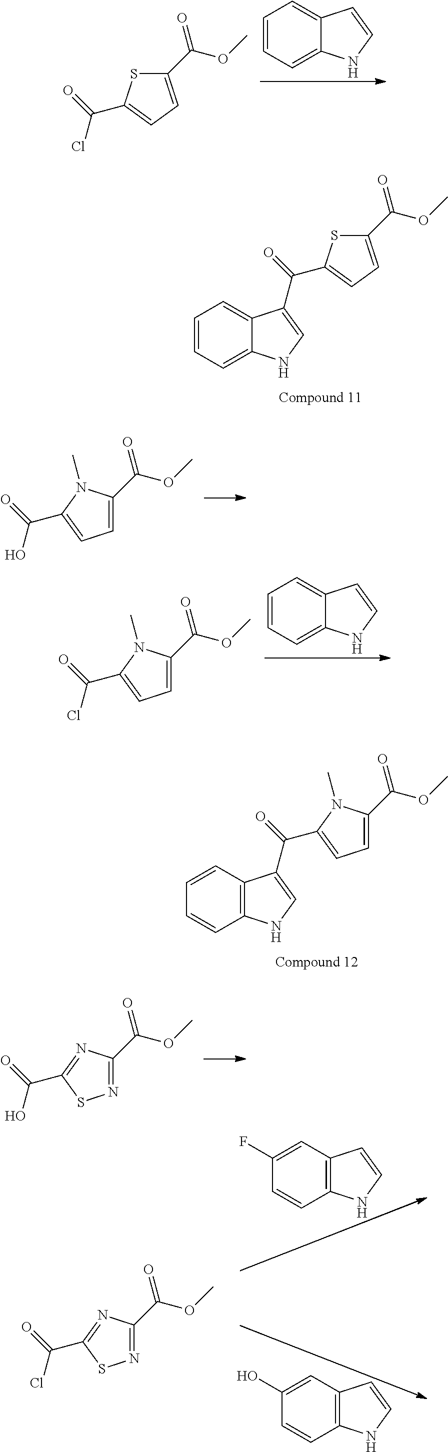

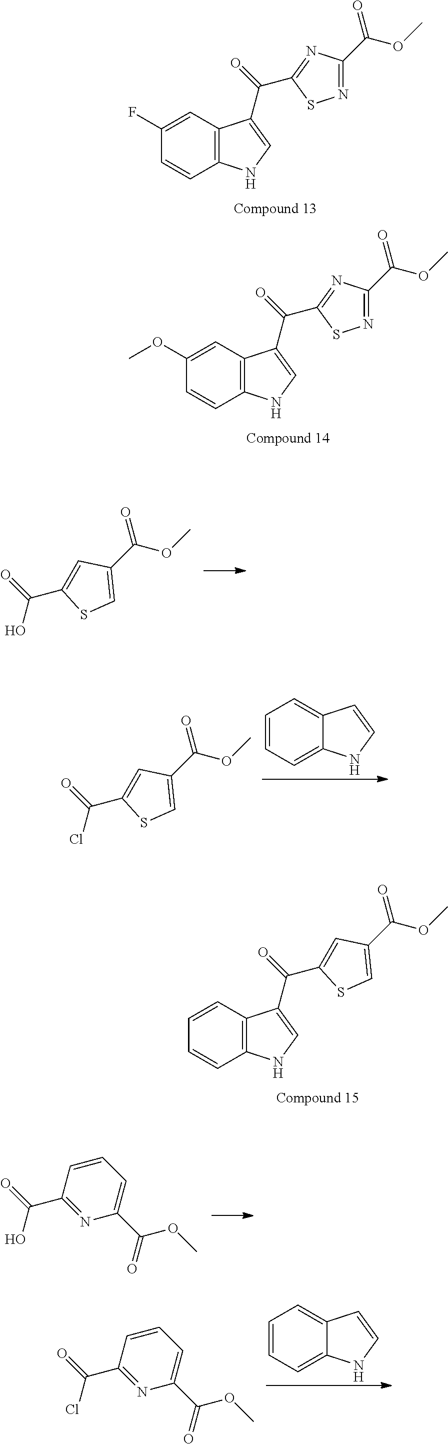

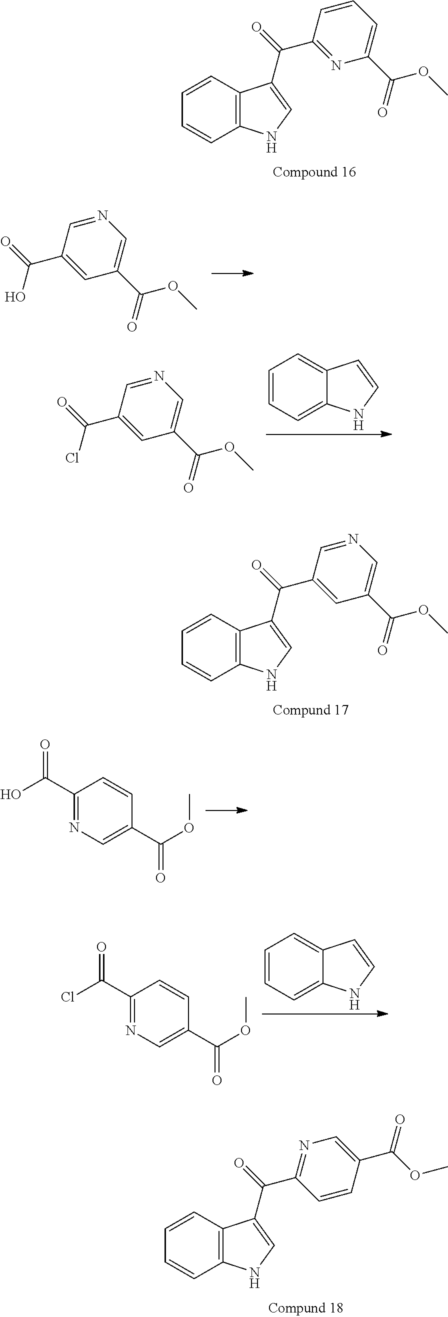

Compounds 9 to 18 were prepared following the same method as that in Embodiment 8, where corresponding acids were respectively used instead of raw material S-2; and other materials were the same as those in Embodiment 8.

Compound 9: MS (ESI) m/z: 271.1 [M+1]⁺.

Compound 10: ¹H NMR (400 MHz, CDCl₃): δ 8.79 (brs, 1H), 8.41-8.43 (m, 1H), 8.24 (S, 1H), 7.98 (d, J=2.8 Hz, 1H), 7.48-7.50 (m, 1H), 7.31-7.37 (m, 2H), 3.37-3.43 (m, 1H), 1.49 (d, J=6.8 Hz, 6H).

Compound 11: ¹H NMR (400 MHz, DMSO-d₆): δ 12.49 (brs, 1H), 9.09 (s, 1H), 8.70 (s, 1H), 8.29-8.34 (m, 1H), 7.58-7.60 (m, 1H), 7.29-7.34 (m, 2H), 3.98 (s, 3H).

Compound 12: ¹H NMR (400 MHz, CDCl₃): δ 8.73 (brs, 1H), 8.50-8.35 (m, 1H), 7.83 (d, J=3.1 Hz, 1H), 7.55-7.41 (m, 1H), 7.43-7.31 (m, 2H), 6.96 (d, J=4.1 Hz, 1H), 6.69 (d, J=4.2 Hz, 1H), 4.25 (s, 3H), 3.90 (s, 3H).

Compound 13: ¹HNMR (400 MHz, DMSO-d₆): δ 12.56 (brs, 1H), 9.06 (s, 1H), 7.94 (dd, J=2.8, 9.6 Hz, 1H), 7.65 (dd, J=4.8, 8.8 Hz, 1H), 7.20 (dt, J=2.8, 9.6 Hz, 1H), 4.00 (s, 3H). MS (ESI) m/z: 306.0 [M+1]⁺.

Compound 14: ¹HNMR (400 MHz, DMSO-d₆): δ 12.43 (brs, 1H), 8.97 (s, 1H), 7.9 (d, J=2.4 Hz, 1H), 7.52 (d, J=8.8 Hz, 1H), 6.97 (dd, J=2.4, 8.8 Hz, 1H), 3.99 (s, 3H), 3.83 (s, 3H). MS (ESI) m/z: 318.0 [M+1]⁺.

Compound 15: ¹H NMR (400 MHz, CDCl₃): δ 9.07 (brs, 1H), 8.41-8.44 (m, 1H), 8.37 (s, 1H), 8.11 (s, 1H), 7.95 (d, J=2.0 Hz, 1H), 7.48 to 7.50 (m, 1H), 7.34-7.37 (m, 2H), 3.94 (s, 3H). MS (ESI) m/z: 286.0 [M+1]⁺.

Compound 16: ¹HNMR (400 MHz, DMSO-d₆): δ 12.22 (brs, 1H), 9.10 (s, 1H), 8.39-8.42 (m, 1H), 8.20 to 8.30 (m, 3H), 7.53 to 7.57 (m, 1), 7.26 to 7.30 (m, 2H), 3.97 (s, 3H). MS (ESI) m/z: 281.0 [M+1]⁺.

Compound 17: ¹H NMR (400 MHz, CDCl₃): δ 9.17 (brs, 1H), 8.43-8.47 (m, 1H), 8.30 (brs, 2H), 7.70 (s, 1H), 7.54-7.56 (m, 2H), 7.38 to 7.40 (m, 2H), 4.09 (s, 3H). MS (ESI) m/z: 286.0 [M+1]⁺.

Compound 18: ¹HNMR (400 MHz, DMSO): δ 12.20 (brs, 1H), 9.23 to 9.24 (m, 1H), 8.76 (s, 1H), 8.51 (dd, J=8.0, J=2.0, 1H), 8.35-8.52 (m, 1H), 8.14 (dd, J=8.4, J=0.8, 1H), 7.53 to 7.56 (m, 1H), 7.25-7.31 (m, 2H), 3.95 (s, 3H). MS(ESI) m/z: 281 [M+1]⁺.

### Embodiment 19. Compounds 19-1 and 19-2

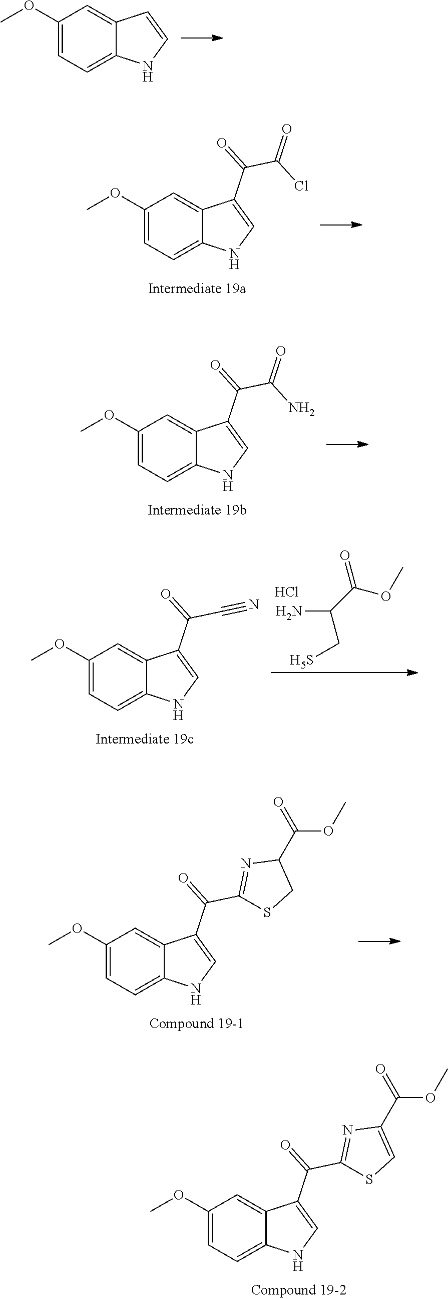

### Synthesis of Intermediate 19a

5-methoxy indole (10 g, 68 mmol) was added to a 250 mL three-necked flask, and methyl t-butyl ether (75 mL) was added and dissolved. The temperature was reduced to −10° C., and oxalyl chloride (9.5 g, 74 mmol) was slowly added dropwise therein while the temperature was controlled at below −5° C. After the dropwise addition was done, the mixture was stirred at low temperature for 1 h, and further stirred at room temperature for 30 min after ice bath was removed. 100 mL petroleum ether was added therein, and the mixture was stirred for 30 min, and filtered. The filter cake was washed with a mixture of petroleum ether and methyl t-butyl ether, and dried to yield intermediate 19a (yield: 15.5 g, 97%). LCMS(ESI) m/z: 234 [M+1]⁺ (the product was diluted with methanol, so that acid chloride was converted to methyl ester).

### Synthesis of Intermediate 19b

At 0° C., intermediate 19a (15.5 g) was added in batch to a mixture of 52.3 g concentrated aqueous ammonia (25%) and 100 mL ethanol; and after the addition was done, the mixture was reacted at 10° C. for 2 h. The reaction mixture was poured into 100 mL ice water, stirred for 30 min, and filtered; and the filter cake was dried to yield a light gray solid, namely, intermediate 19b (10.5 g).

LCMS(ESI) m/z: 219 [M+1]⁺.

### Synthesis of Intermediate 19c

Intermediate 19b (10 g, 45.8 mmol) was suspended in 150 mL ethyl acetate, and pyridine (10.87 g, 137.5 mmol) was added therein. The temperature was reduced to below 10° C., and trifluoroacetic anhydride (14.43 g, 68.7 mmol) was slowly added dropwise therein over about 30 min. After the dropwise addition was done, the reaction was continued at 10° C. for 2 h. The reaction was poured into 100 mL ice water, and extracted 2 times with ethyl acetate. The organic phases were combined, washed 2 times with saturated sodium bicarbonate and 2 times with 0.5N diluted HCl; dried using anhydrous sodium sulfate, filtered, and concentrated under reduced pressure to yield 8.8 g of a crude solid. The crude solid was washed with a mixed solvent of ethyl acetate:dichloromethane=5:1, and filtered to yield intermediate 19c (yield: 7.2 g, 78%). ¹H NMR (400 MHz, CDCl3): δ 12.76 (bis, 1H), 8.53 (s, 1H), 7.48-7.51 (m, 2H), 6.99 (dd, J=8.8 Hz, J=2.4 Hz, 1H), 3.80 (s, 3H). MS (ESI) m/z: 201.0 [M+1]+.

### Synthesis of Compound 19-1

Intermediate 19c (2 g, 10 mmol) was dissolved in N,N′-dimethylformamide (15 mL), and L-cysteine methyl ester hydrochloride (1.72 g, 10 mmol) and DBU (152 mg, 1 mmol) were added therein. The temperature was raised to 40° C. to react for 3 h. After cooling to room temperature, the reaction was dropped into 80 mL of ice-diluted HCl (containing 0.1 mmol HC1), stirred for 20 min, and filtered. The filter cake was pressed dry, washed with a small amount of dichloromethane, and dried to yield intermediate 19-1 (yield: 3.1 g, 97%). ¹H NMR (400 MHz, CDCl₃): δ 8.78 (brs, 1H), 8.71 (d, J=2.8 Hz, 1H), 7.97 (d, J=2.8 Hz, 1H), 7.33 (d, J=8.8 Hz, 1H), 6.97 (dd, J=8.8 Hz, J=2.8 Hz, 1H), 5.48 (t, J=8.8 Hz, 1H), 3.92 (s, 3H), 3.89 (t, 3H), 3.61 (d, J=9.6 Hz, 2H). MS (ESI) m/z: 319.0 [M+1]⁺.

### Synthesis of Compound 19-2

Compound 19-1 (2.6 g, 8.16 mmol) was dissolved in N,N-dimethylformamide (30 mL) and air was bubbled through the reaction at 80° C. for 12 h. The reaction was dropped into ice water, stirred for 20 min, and filtered. The filter cake was washed with water and dried to yield compound 19-2 (yield: 2.5 g, 96%). ¹HNMR (400 MHz, CDCl₃): δ 9.23 (d, J=3.6 Hz, 1H), 9.02 (brs, 1H), 8.44 (s, 1H), 8.05 (d, J=2.4 Hz, 1H), 7.37 (d, J=8.8 Hz, 1H), 6.99 (dd, J=8.8 Hz, J=2.4 Hz, 1H), 4.03 (s, 3H), 3.95 (s, 3H). MS (ESI) m/z: 317.0 [M+1]⁺.

### Embodiment 20. Compounds 20-1 and 20-2

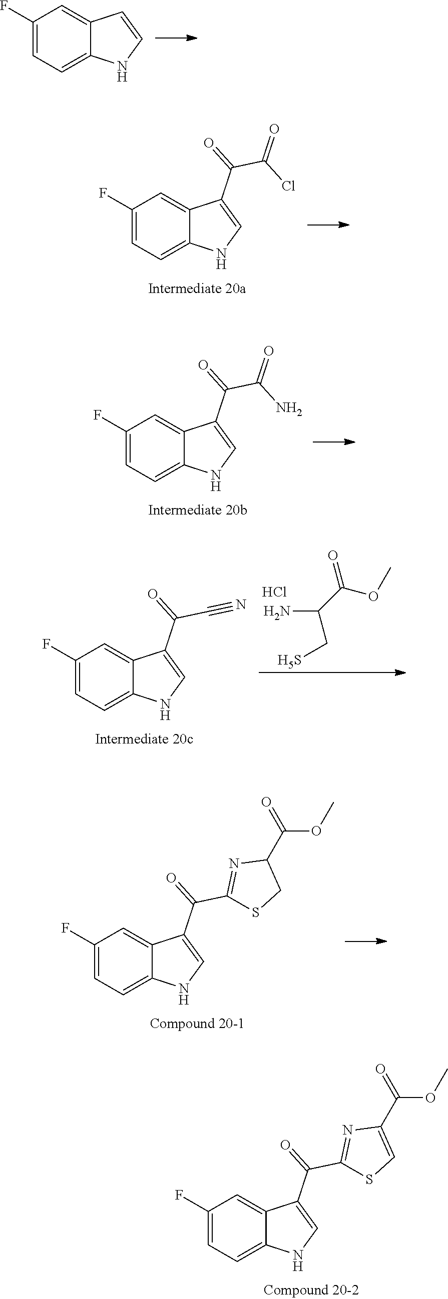

The synthetic route of compound 20-1 and compound 20-2 was the same as that in Embodiment 19, where the starting material 5-fluoroindole was used in place of 5-methoxyindole. Related structure identification data was as follows:

Intermediate 20b: MS(ESI) m/z: 207.2 [M+1]⁺.

Intermediate 20c: ¹H NMR (400 MHz, DMSO-d₆): δ 12.94 (brs, 1H), 8.68 (s, 1H), 7.70 (dd, J=2.4, 9.2 Hz, 1H), 7.62 (dd, J=4.4, 8.8 Hz, 1H), 7.24 (dt, J=2.4, 9.2 Hz, 1H). MS(ESI) m/z: 189 [M+1]⁺.

Compound 20-1: ¹H NMR (400 MHz, DMSO-d₆): δ 12.42 (brs, 1H), 8.69 (d, J=3.2 Hz, 1H), 7.87 (dd, J=2.4, 8.8 Hz, 1H), 7.59 (dd, J=4.4, 8.8 Hz, 1H), 7.16 (dt, J=2.4, 9.2 Hz, 1H), 5.67 (dd, J=8.4, 10.0 Hz, 1H), 3.92 (s, 3H), 3.68 (dd, J=11.2, 10.0 Hz, 1H), 3.55 (dd, J=8.4, 11.2 Hz, 1H). MS(ESI) m/z: 307 [M+1]⁺.

Compound 20-2: ¹H NMR (400 MHz, DMSO-d₆): δ 12.48 (brs, 1H), 9.13 (s, 1H), 8.89 (s, 1H), 7.97 (dd, J=2.4, 9.6 Hz, 1H), 7.62 (dd, J=4.4, 8.8 Hz, 1H), 7.17 (dt, J=2.4, 9.2 Hz, 1H), 3.92 (s, 3H). MS(ESI) m/z: 305 [M+1]⁺.

### Embodiment 21. Compound 21

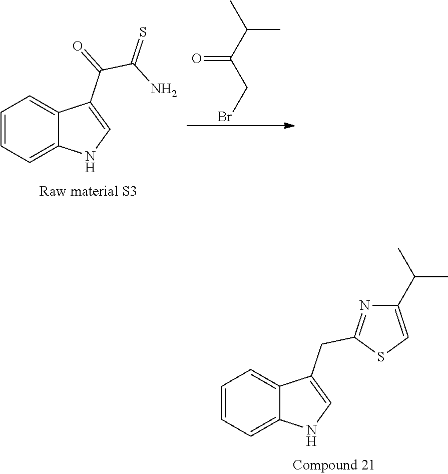

### Synthesis of Compound 21

1-bromo-3-methyl-2-butanone (0.8 g, 4.89 mmol) was dissolved in ethanol (25 mL), and raw material S3 (1.0 g, 4.89 mmol) was added therein with stirring. The mixture was heated to 80° C. and allowed to react for 2 h. The reaction was cooled to room temperature and filtered. The filter cake was washed with ethanol to yield compound 21 (yield: 0.6 g, 45%). ¹HNMR (400 MHz, DMSO-d₆): δ 12.22 (brs, 1H), 9.10 (d, J=3.2 Hz, 1H), 8.31 to 8.33 (m, 1H), 7.77 (s, 1H), 7.57 to 7.59 (m, 1H), 7.25 to 7.31 (m, 2H), 3.16 to 3.23 (m, 1H), 1.36 (d, J=6.8 Hz, 6H).

### Embodiment 22. Compound 22

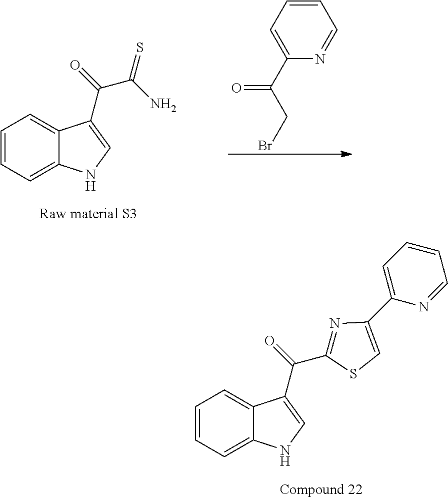 [SMILES: NC(=S)C(=O)c1c[nH]c2ccccc12 ; O=C(CBr)c1ccccn1 ; O=C(c1nc(-c2ccccn2)cs1)c1c[nH]c2ccccc12]

The synthesis for compound 22 was the same as the synthesis for compound 21; and raw material S3 (1.0 g, 4.89 mmol) was used to prepare compound 22 (yield: 1.2 g, 80%). ¹HNMR (400 MHz, DMSO-d₆): δ 12.30 (brs, 1H), 9.30 (s, 1H), 8.69 (dd, J=1.2, 4.2 Hz, 1H), 8.65 (s, 1H), 8.34-8.36 (m, 1H), 8.32 (d, J=1.2 Hz, 1H), 8.01 (dt, J=2.0, 7.2 Hz, 1H), 7.60-7.62 (m, 1H), 7.44-7.47 (m, 1H), 7.30-7.34 (m, 2H).

### Embodiment 23. Compound 23

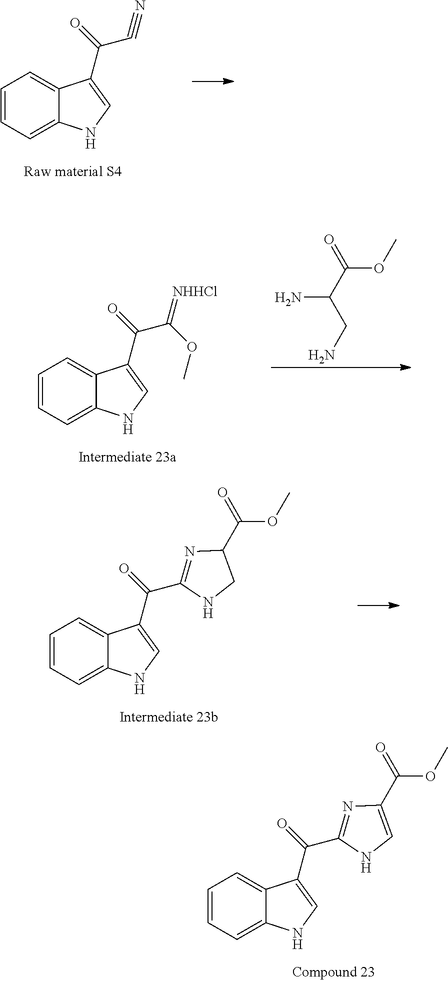

### Synthesis of Intermediate 23a

Raw material S4 (4.0 g, 23.5 mmol) was dissolved in methanol (50 mL); the temperature was reduced to below 0° C., and dry hydrogen chloride gas was continuously charged therein for 8 h and stopped. The mixture was sealed and stirred overnight and filtered to yield 5.4 g of a yellow solid, namely, intermediate 23a, which was directly used for subsequent reaction.

### Synthesis of Intermediate 23b

Intermediate 23a (5.4 g, 19.6 mmol) was dissolved in acetonitrile (15 mL); methyl 2,3-diaminopropionate hydrochloride (3.7 g, 19.6 mmol) was added and triethylamine (10 g, 98 mmol) was dropped therein; and the mixture was reacted at reflux for 5 h. The solvent was removed under reduced pressure; the residue was dissolved with water and dichloromethane, and the layers were separated. The aqueous phase was extracted 2 times with dichloromethane, and the organic phases were combined, washed with saturated brine, dried over anhydrous sodium sulfate, and concentrated under reduced pressure to yield a crude product. The crude product was purified using silica gel column chromatography to yield intermediate 23b (yield: 2.4 g, 45%). ¹H NMR (400 MHz, CDCl₃): δ 9.19 (s, 1H), 8.91 (d, J=2.8 Hz, 1H), 8.44 (dd, J=6.8 Hz, J=1.6 Hz, 1H), 7.41-7.43 (m, 1H), 7.30-7.36 (m, 2H), 4.67 (brs, 1H), 4.18 (d, J=7.6 Hz, 2H), 3.82 (s, 3H), 1.87 (brs, 1H). MS (ESI) m/z: 272 [M+1]⁺.

### Synthesis of Compound 23

Intermediate 23b (1.2 g, 4.42 mmol) was dissolved in DMF (20 mL); sodium hydroxide (530 mg, 13.3 mmol) was added therein; and air was charged at 60° C. to react for 3 h with stirring. The reaction was cooled, poured into ice water, and extracted 3 times with ethyl acetate. The organic phase was washed with saturated brine, dried over anhydrous sodium sulfate, and concentrated under reduced pressure to yield a crude product. The crude product was washed with a mixed solvent of PE:EA=2:1 to yield compound 23 (yield: 960 mg, 81%). ¹HNMR (400 MHz, DMSO): δ 13.69 (brs, 1H), 12.20 (s, 1H), 9.15 (s, 1H), 8.32-8.36 (m, 1H), 8.03 (s, 1H), 7.55-7.59 (m, 1H), 7.24-7.30 (m, 2H), 3.83 (s, 3H). MS(ESI) m/z: 270 [M+1]⁺.

### Embodiment 24. Compound 24

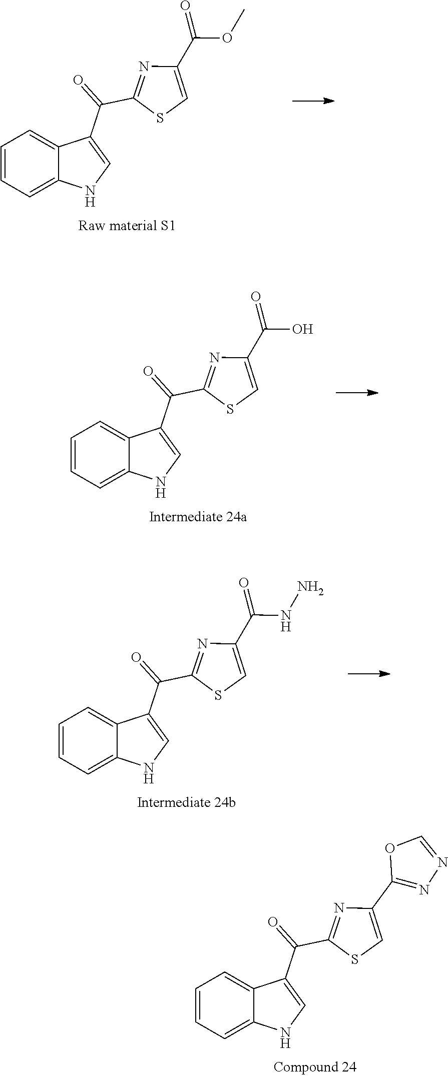 [SMILES: COC(=O)c1csc(C(=O)c2c[nH]c3ccccc23)n1 ; O=C(O)c1csc(C(=O)c2c[nH]c3ccccc23)n1 ; NNC(=O)c1csc(C(=O)c2c[nH]c3ccccc23)n1 ; O=C(c1nc(-c2nnco2)cs1)c1c[nH]c2ccccc12]

### Synthesis of Intermediate 24a

Raw material S1 (2.86 g, 10 mmol) was dissolved in a mixed solvent of THF/MeOH/H₂O (16/15/15 mL) and stirred overnight at room temperature. The reaction was adjusted to pH=4-5 with 4N hydrochloric acid and then filtered. The filter cake was washed with water and dried in vacuo to yield intermediate 24a (yield: 2.6 g, 96%). MS(ESI) m/z: 271[M−1]⁻.

### Synthesis of Intermediate 24b

Intermediate 24a (1.36 g, 5 mmol) was dissolved in THF (20 mL), 2 drops of DMF was added, and oxalyl chloride (755 mg, 6 mmol) was added dropwise therein. The mixture was reacted at room temperature for 2 h and then was concentrated to dryness under reduced pressure. The residue was dissolved in THF (20 mL), and then the solution was added dropwise to 80% hydrazine (2 mL, 57 mmol) and stirred overnight. The reaction was concentrated under reduced pressure to 5 mL and filtered. The filter cake was washed with THF and dried to yield intermediate 24b (yield: 1.38 g, 97%).

### Synthesis of Compound 24

A mixture of intermediate 24b (1.0 g, 3.5 mmol), p-toluenesulfonic acid monohydrate (20 mg) and trimethyl orthoformate (5 mL) was heated to 80° C., stirred overnight, poured into ice water, and filtered. The filter cake was washed with ethyl acetate, and dried to yield compound 24 (yield: 280 mg, 27%). ¹H NMR (400 MHz, DMSO-d₆): δ 12.45 (brs, 1H), 9.43 (s, 1H), 9.15 (s, 1H), 8.95 (s, 1H), 8.32 (m, 1H), 7.61 (m, 1H), 7.32 (m, 2H). MS(ESI) m/z: 297 [M+1].

### Embodiment 25. Compounds 25-1 and 25-2

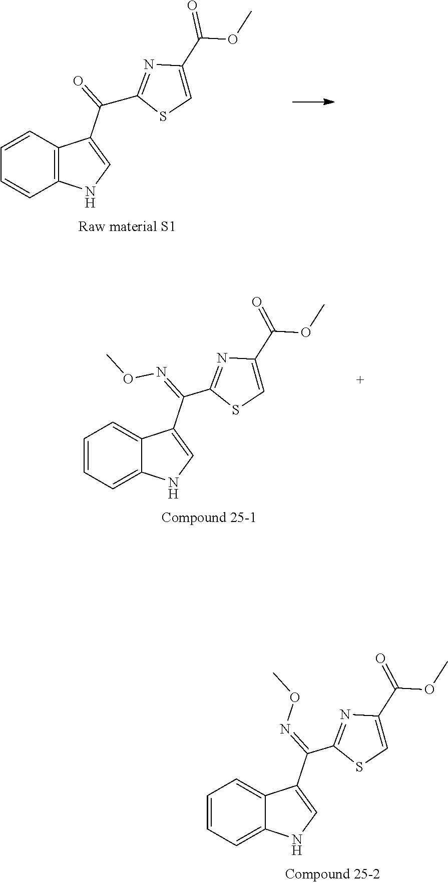 [SMILES: COC(=O)c1csc(C(=O)c2c[nH]c3ccccc23)n1 ; CO/N=C(/c1nc(C(=O)OC)cs1)c1c[nH]c2ccccc12 ; CO/N=C(\c1nc(C(=O)OC)cs1)c1c[nH]c2ccccc12]

### Synthesis of Compounds 25-1 and 25-2

Raw material S1 (1.0 g, 3.5 mmol) was dissolved in pyridine (15 mL), and methoxyamine hydrochloride (1.75 g, 21 mmol) was added therein. The mixture was heated to 90° C. and reacted for 24 h, cooled to room temperature, diluted in water, and extracted 2 times with ethyl acetate. The organic phase was washed 2 times with IN hydrochloric acid and washed with saturated brine, dried using anhydrous sodium sulfate, and concentrated under reduced pressure to yield a crude product. The crude product was purified with silica gel column chromatography (petroleum ether:ethyl acetate=20:1 to 5:17) to yield compound 25-1 (410 mg) and compound 25-2 (300 mg), 64.3% yield.

Compound 25-1: ¹H NMR (400 MHz, CDCl₃): δ 8.54 (d, J=3.2 Hz, 1H), 8.51 (brs, 1H), 8.42 (s, 1H), 8.37-8.39 (m, 1H), 7.41-7.43 (m, 1H), 7.25-7.29 (m, 2H), 4.32 (s, 3H), 4.00 (s, 3H). MS(ESI) m/z: 316 [M+1]⁺.

Compound 25-2: ¹HNMR (400 MHz, CDCl3): δ 8.94 (bis, 1H), 8.24 (s, 1H), 7.80 (d, J=2.8 Hz, 1H), 7.40 (d, J=7.6 Hz, 1H), 7.29-7.31 (m, 1H), 7.14-7.18 (m, 1H), 7.09-7.13 (m, 1H), 4.16 (s, 3H), 3.92 (s, 3H). MS(ESI) m/z: 316 [M+1]⁺.

### Embodiment 26. Compounds 26-1 and 26-2

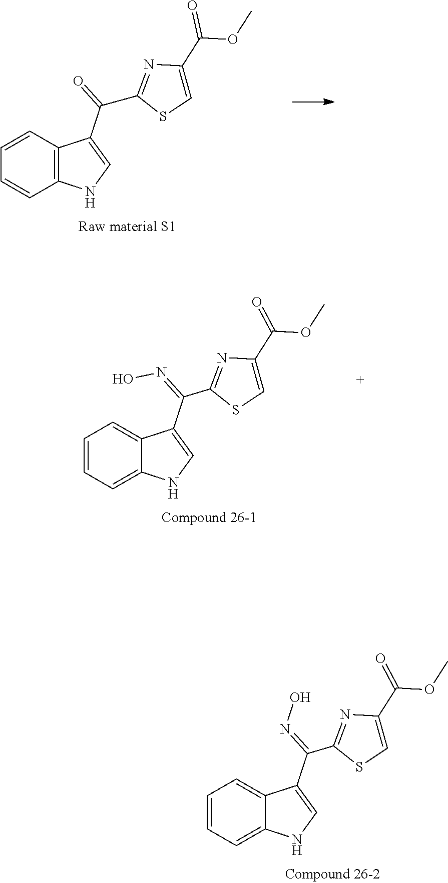 [SMILES: COC(=O)c1csc(C(=O)c2c[nH]c3ccccc23)n1 ; COC(=O)c1csc(/C(=N/O)c2c[nH]c3ccccc23)n1 ; COC(=O)c1csc(/C(=N\O)c2c[nH]c3ccccc23)n1]

### Synthesis of Compounds 26-1 and 26-2

The method was the same as the synthesis method for compounds 25-1 and 25-2; and raw material S1 (324 mg, 1.13 mmol) and hydroxylamine hydrochloride (696 mg, 10 mmol) were used to prepare compounds 26-1 and 26-2 (yield: 149 mg, 44%).

Compound 26-1: ¹HNMR (400 MHz, CDCl₃): δ 9.00 (s, 1H), 8.26 (s, 1H), 8.19 (d, J=8.0 Hz, 1H), 7.80 (d, J=2.8 Hz, 1H), 7.47 (d, J=8.0 Hz, 1H), 7.09-7.19 (m, 2H), 3.93 (s, 3H). MS(ESI) m/z: 302 [M+1]⁺.

Compound 26-2: ¹H NMR (400 MHz, CDCl₃): δ 8.58 (s, 1H), 8.45 (s, 1H), 8.27 (d, J=3.2 Hz, 1H), 7.40 (dd, J=7.2 Hz, J=1.6 Hz, 1H), 7.33 (d, J=8.0 Hz, 1H), 7.20-7.28 (m, 2H), 4.01 (s, 3H).

MS(ESI) m/z: 302 [M+1]⁺.

### Embodiment 27. Compound 27

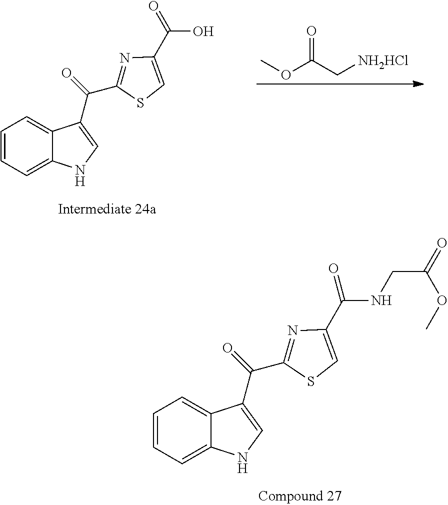

Glycine methyl ester hydrochloride (753 mg, 6 mmol), HATU (2.26 g, 6 mmol), and DIEA (2.3 g, 10 mmol) was added to a solution of intermediate 24a (1.36 g, 5 mmol) in DMF (20 mL), and stirred at room temperature for 2 h. The reaction mixture was poured into 100 mL of ice water, and filtered; and the filter cake was washed with ethyl acetate and dried to yield compound 27 (yield: 1.45 g, 84.5%). ¹H NMR (400 MHz, DMSO-d₆): δ 12.40 (d, J=2.0 Hz, 1H), 9.43 (d, J=3.2 Hz, 1H), 9.29 (t, J=2.4 Hz, 1H), 8.66 (s, 1H), 8.32-8.35 (m, 1H), 7.58-7.60 (m, 1H), 7.27-7.34 (m, 2H), 4.13 (d, J=6.4 Hz, 2H), 3.70 (s, 3H).

### Embodiment 28. Compound 28

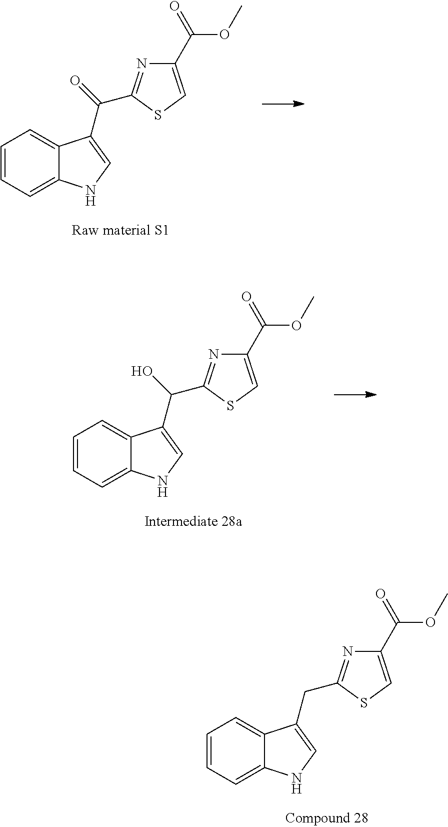 [SMILES: COC(=O)c1csc(C(=O)c2c[nH]c3ccccc23)n1 ; COC(=O)c1csc(C(O)c2c[nH]c3ccccc23)n1 ; COC(=O)c1csc(Cc2c[nH]c3ccccc23)n1]

### Synthesis of Intermediate 28a

Raw material S1 (7 g, 24 mmol) was dissolved in a mixed solvent of THF (42 mL) and methanol (168 mL). The temperature was reduced to 0° C. with an ice bath and then sodium borohydride (4.6 g, 12.2 mmol) in batch was slowly added therein. The ice-salt bath was removed; the temperature was increased to room temperature, and the reaction was conducted for 1 h. The reaction was poured into ice water, filtered, and the filter cake was washed with methanol and dried to yield intermediate 28a (yield: 6.8 g, 98%). ¹H NMR (400 MHz, DMSO-d₆): δ 11.07 (s, 1H), 8.46 (s, 1H), 7.48 (d, J=8.0 Hz, 1H), 7.37 (d, J=8.0 Hz, 1H), 7.34 (d, J=2.4 Hz, 1H), 7.07 (dt, J=0.8, 8.0 Hz, 1H), 6.96 (dt, J=0.8, 8.0 Hz, 1H), 6.68 (d, J=4.0 Hz, 1H), 6.18 (d, J=4.0 Hz, 1H), 3.77 (s, 3H). MS (ESI) m/z: 291.0 [M+1]⁺.

### Synthesis of Compound 28

Intermediate 28a (3 g, 10.4 mmol) was dissolved in methanol (25 mL), and zinc powder (2 g, 31.2 mmol) was added therein with stirring. The mixture was refluxed at 100° C. under nitrogen protection for 1 h. Then, the reaction was added dropwise to ice water and filtered to yield 1.8 g of a crude product. A portion of the crude product (200 mg) was purified using silica gel column chromatography (PE/EA=4/1 to 2/1) to yield compound 28 (20 mg). ¹HNMR (400 MHz, DMSO-d₆): δ 11.06 (s, 1H), 8.32 (s, 1H), 7.39-7.44 (m, 3H), 7.10 (dt, J=1.1, 8.0 Hz, 1H), 6.98 (dt, J=1.1, 8.0 Hz, 1H), 4.05 (s, 2H), 3.81 (s, 3H). MS (ESI) m/z: 275.0 [M+1]⁺.

### Embodiment 29. Compound 29

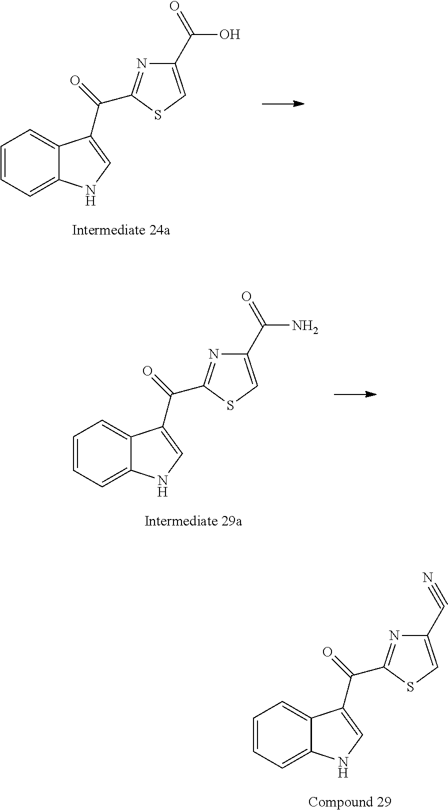 [SMILES: O=C(O)c1csc(C(=O)c2c[nH]c3ccccc23)n1 ; NC(=O)c1csc(C(=O)c2c[nH]c3ccccc23)n1 ; N#Cc1csc(C(=O)c2c[nH]c3ccccc23)n1]

### Synthesis of Intermediate 29a

Intermediate 24a (1.36 g, 5 mmol) was dissolved in THF (20 mL), 2 drops of DMF was added, and oxalyl chloride (755 mg, 6 mmol) was added dropwise therein. The mixture was reacted at room temperature for 2 h and then was concentrated to dryness under reduced pressure. The residue was dissolved in THF (20 mL); and the solution was added dropwise to concentrated aqueous ammonia (10 mL) and stirred overnight. The reaction was concentrated to 5 mL under reduced pressure, and filtered; and the filter cake was washed with THF and dried to yield intermediate 29a (yield: 1.3 g, 95%). ¹H NMR (400 MHz, DMSO-d₆): δ 12.27 (s, 1H), 9.52 (s, 1H), 8.61 (s, 1H), 8.31-8.35 (m, 1H), 7.57-7.60 (m, 1H), 8.28 (s, 1H), 7.81 (s, 1H), 7.26-7.34 (m, 2H). MS (ESI) m/z: 272.0 [M+1]⁺.

### Synthesis of Compound 29

Intermediate 29a (17 g, 62.66 mmol) was dissolved in ethyl acetate (250 mL) and pyridine (14.87 g, 187.9 mmol) was added therein. At room temperature, trifluoroacetic anhydride (19.7 g, 93.99 mmol) was added dropwise therein. The mixture was stirred at room temperature for 4 h, concentrated to dryness under reduced pressure, and recrystallized from ethyl acetate to yield compound 29 (yield: 14 g, 88%). ¹HNMR (400 MHz, DMSO-d₆): δ 12.44 (s, 1H), 9.15 (s, 1H), 9.03 (d, J=3.6 Hz, 1H), 8.28-8.31 (m, 1H), 7.57-7.62 (m, 1H), 7.29-7.34 (m, 2H). MS (ESI) m/z: 254.0 [M+1]⁺.

### Embodiment 30. Compounds 30-1 and 30-2

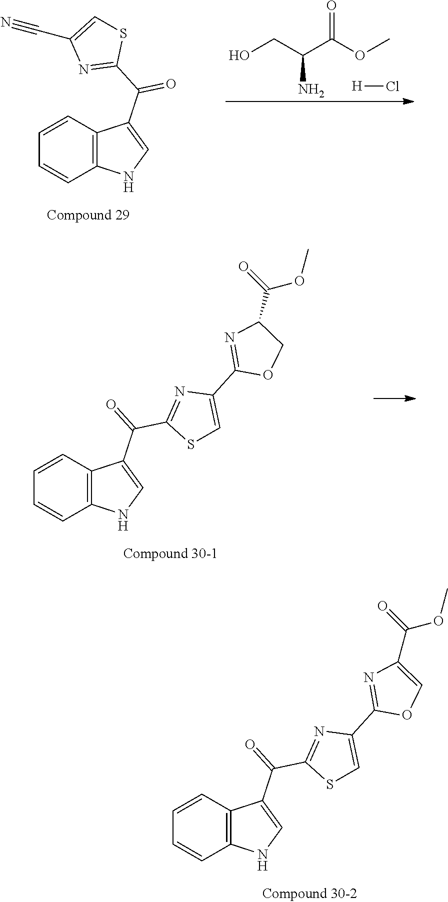

### Synthesis of Compound 30-1

Compound 29 (1 g, 3.9 mmol) was dissolved in methanol (100 mL), and displacement with nitrogen gas was performed three times; a sodium methoxide solution (sodium metal 0.23 g, 10 mmol, 50 mL methanol) was added dropwise therein. The mixture was stirred at room temperature for 4 h; and a solution of L-serine methyl ester hydrochloride (1.8 g, 11.6 mmol) in methanol (50 mL) was added dropwise therein. The mixture was heated to 55° C., stirred for 2 h, poured into ice water, and filtered to yield a crude product. The crude product was purified using silica gel column chromatography (PE:EA=1:1) to yield compound 30-1 (yield: 0.4 g, 29%). ¹H NMR (400 MHz, DMSO-d₆): δ 12.33 (s, 1H), 9.10 (d, J=2.9 Hz, 1H), 8.73 (s, 1H), 8.44-8.21 (m, 1H), 7.69-7.49 (m, 1H), 7.40-7.21 (m, 2H), 5.06 (dd, J=10.0, 8.0 Hz, 1H), 4.76-4.57 (m, 2H), 3.74 (s, 3H). MS (ESI) m/z: 356.0 [M+1]⁺.

### Synthesis of Compound 30-2

Compound 30-1 (200 mg, 0.56 mmol) was dissolved in tetrahydrofuran (50 mL), and manganese dioxide (1000 mg, 11.56 mmol) was added therein. The mixture was refluxed overnight, cooled, and filtered. The filtrate was concentrated to dryness under reduced pressure to yield a crude product. The crude product was purified using silica gel column chromatography (PE:EA=2:1) to yield compound 30-2 (yield: 25 mg, 12%). ¹H NMR (400 MHz, DMSO-d₆): δ 12.37 (s, 1H), 9.17 (d, J=2.7 Hz, 1H), 9.09 (s, 1H), 8.90 (s, 1H), 8.37-8.29 (m, 1H), 7.66-7.59 (m, 1H), 7.38-7.26 (m, 2H), 3.89 (s, 3H). MS (ESI) m/z: 354 [M+1]⁺.

### Embodiment 31. Compounds 31-1 and 31-2

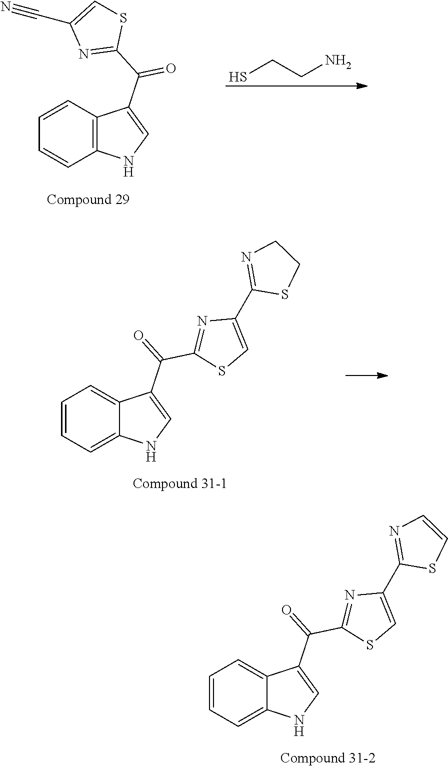

Compound 31-1: the method was the same as that for compound 30-1. ¹H NMR (400 MHz, DMSO-d₆): δ 12.41 (s, 1H), 9.08 (d, J=3.1 Hz, 1H), 8.63 (s, 1H), 8.42-8.24 (m, 1H), 7.68-7.49 (m, 1H), 7.31 (m, 2H), 4.47 (t, J=8.5 Hz, 2H), 3.48 (t, 8.5 Hz, 2H). MS (ESI) m/z: 314.0 [M+1]⁺.

Compound 31-2: the method was the same as that for compound 30-2. ¹H NMR (400 MHz, DMSO-d₆): δ 12.41 (s, 1H), 9.13 (d, J=3.2 Hz, 1H), 8.64 (s, 1H), 8.37-8.30 (m, 1H), 8.01 (d, J=3.2 Hz, 1H), 7.91 (d, J=3.2 Hz, 1H), 7.65-7.57 (m, 1H), 7.35-7.27 (m, 2H). MS (ESI) m/z: 312.0 [M+1]⁺.

### Embodiment 32. Compounds 32-1 and 32-2

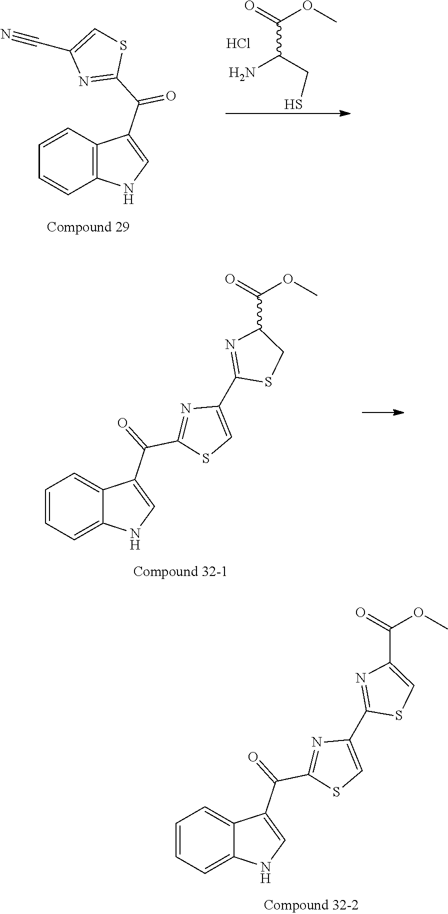

Compound 32-1: the method was the same as that for compound 30-1. ¹H NMR (400 MHz, DMSO-d₆): δ 12.43 (s, 1H), 9.04 (s, 1H), 8.70 (s, 1H), 8.29-8.344 (m, 1H), 7.57-7.60 (m, 1H), 7.21-7.34 (m, 2H), 5.48 (dd, J=9.2, 8.4 Hz, 1H), 3.78 (dd, J=6.0, 11.6 Hz, 1H), 3.75 (s, 3H), 3.67 (dd, J=11.6, 8.4 Hz, 1H). MS (ESI) m/z: 372.0 [M+1]⁺.

Compound 32-2: the method was the same as that for compound 30-2. MS (ESI) m/z: 370.0 [M+1]⁺.

### Embodiment 33. Compounds 33-1 and 33-2

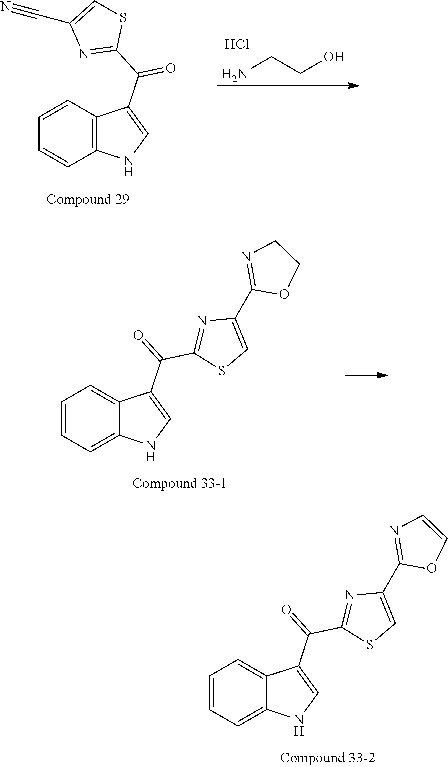

Compound 33-1: the method was the same as that for compound 30-1. MS (ESI) m/z: 298.0 [M+1]⁺.

Compound 33-2: the method was the same as that for compound 30-2. ¹H NMR (400 MHz, DMSO-d₆): δ12.41 (s, 1H), 9.13 (d, J=3.2 Hz, 1H), 8.64 (s, 1H), 8.37-8.30 (m, 1H), 7.65-7.57 (m, 1H), 7.52 (brs, 1H), 7.35-7.27 (m, 2H), 7.11 (brs, 1H). MS (ESI) m/z: 296.0 [M+1]⁺.

### Embodiment 34. Compound 34

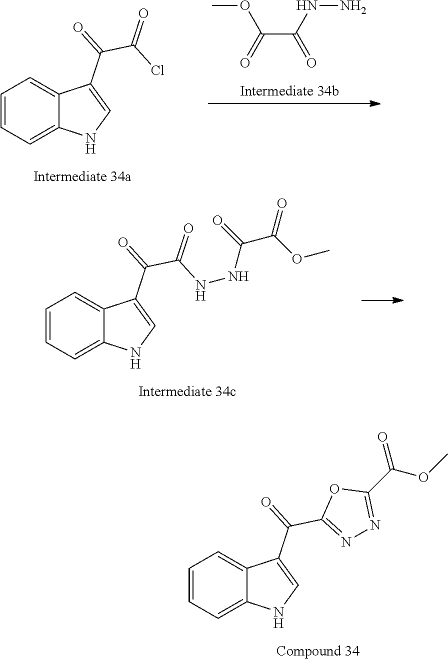

For synthesis of intermediate 34a, please refer to J. Am. Chem. Soc., 2002, 124(44). 13179-13184.

For synthesis of intermediate 34b, refer to J. Med. Chem., 1961, 4, 259-296.

### Synthesis of Intermediate 34c

Compound 34b (1.18 g, 10 mmol) and triethylamine (3.03 g, 30 mmol) were dissolved in dichloromethane (15 mL); and at 0° C., a solution of compound 34a (2.07 g, 10 mmol) in dichloromethane (10 mL) was added dropwise therein. The reaction mixture was stirred at room temperature overnight. The reaction was diluted with 30 mL water and extracted 3 times with dichloromethane. The organic phases were combined, dried using anhydrous sodium sulfate, filtered, and concentrated under reduced pressure to yield intermediate 34c (yield: 2.8 g, 97%). MS (ESI) m/z: 290.0 [M+1]⁺.

### Synthesis of Compound 34

Intermediate 34c (5 g, 17.286 mmol) was dissolved in DMF (200 mL), and triethylamine (5.2 g, 51.86 mmol) was added therein with stirring; then THF (100 mL) was added therein. P-toluensulfonyl chloride (9.88 g, 51.86 mmol) dissolved in dichloromethane (50 mL) was slowly added dropwise therein over 1 h under nitrogen protection; and then the mixture was reacted at room temperature overnight. After dichloromethane and THF were removed by concentration under reduced pressure, the reaction was added dropwise to ice water, stirred, and filtered to yield a crude product. The crude product was purified using silica gel column chromatography (dichloromethane/methanol=50/1 to 10/1) to yield compound 34 (yield: 0.5 g, 10%). ¹H NMR (400 MHz, DMSO-d₆): δ 12.53 (brs, 1H), 8.90 (s, 1H), 8.27-8.29 (m, 1H), 7.60-7.62 (m, 1H), 7.32-7.37 (m, 2H), 4.02 (s, 3H). MS (ESI) m/z: 272.1 [M+1]⁺.

### Embodiment 35. Compound 35

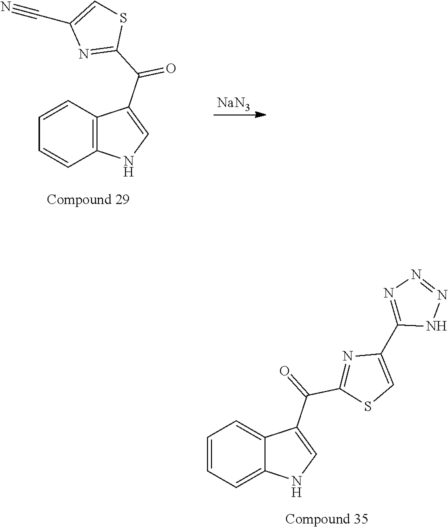 [SMILES: N#Cc1csc(C(=O)c2c[nH]c3ccccc23)n1 ; O=C(c1nc(-c2nnn[nH]2)cs1)c1c[nH]c2ccccc12 ; "NaN3"]

Compound 29 (2 g, 7.9 mmol) was added to a sealed reactor; DMF (30 mL) was added therein and stirred; and ammonium chloride (0.49 g, 9.2 mmol) and sodium azide (0.6 g, 9.2 mmol) were added. The reactor was sealed, and the mixture was reacted in an oil bath at 120° C. overnight. The reaction was cooled to room temperature, added dropwise to 200 mL ice water, and extracted with ethyl acetate (150 mL). The aqueous phase was adjusted to an acidic pH with 2N hydrochloric acid. The solid was precipitated and then filtered, washed with water, and dried to yield compound 35 (1.8 g, 77%). ¹H NMR (400 MHz, DMSO-d₆): δ 12.50 (s, 1H), 9.48 (d, J=3.6 Hz, 1H), 8.88 (s, 1H), 8.36-8.34 (m, 1H), 7.62-7.60 (m, 1H), 7.34-7.31 (m, 2H). MS (ESI) m/z: 297.0 [M+1]⁺.

### Embodiment 36. Compound 36

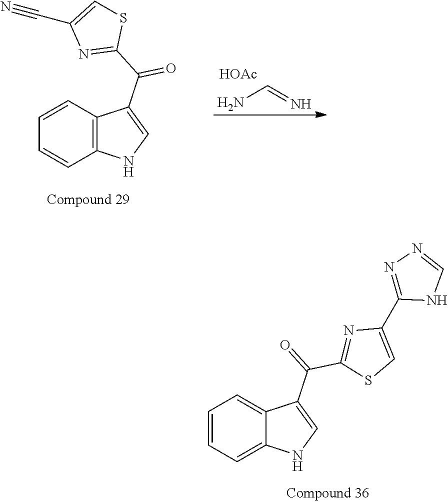

Compound 29 (0.5 g, 1.7 mmol) was suspended in 10 mL ethylene glycol methyl ether, and 2 mL acetic acid and formamidine acetate (0.215 g, 2.07 mmol) were added therein. The mixture was refluxed in an oil bath under nitrogen protection for 24 h. The reaction was distilled under reduced pressure, and the crude product was purified using silica gel column chromatography (DCM/methanol=200/1 to 20/1) to yield compound 35 (yield: 0.32 g, 55%). ¹H NMR (400 MHz, DMSO-d₆): δ 12.41 (s, 1H), 10.6 (s, 1H), 10.05 (s, 1H), 9.55 (s, 1H), 8.72 (s, 1H), 8.32-8.34 (m, 1H), 7.58-7.59 (m, 1H), 7.28-7.33 (m, 2H). MS (ESI) m/z: 296.0 [M+1]⁺.

### Embodiment 37. Compound 37

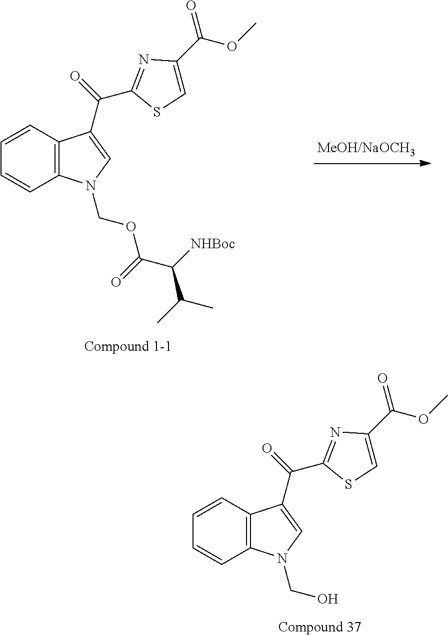 [SMILES: COC(=O)c1csc(C(=O)c2cn(COC(=O)[C@@H](NC(=O)OC(C)(C)C)C(C)C)c3ccccc23)n1 ; COC(=O)c1csc(C(=O)c2cn(CO)c3ccccc23)n1]

Compound 1-1 (500 mg, 0.97 mmol) was dissolved in methanol (2 mL), and a 0.1N sodium methoxide solution (2 mL) was added dropwise therein. The mixture was reacted at room temperature overnight, and filtered. The solid was washed with methanol and dried to yield compound 37 (yield: 153 mg, 50%/). ¹H NMR (400 MHz, CDCl₃): δ 9.25 (s, 1H), 8.93 (s, 1H), 8.35 (d, J=7.6 Hz, 1H), 7.81 (d, J=7.6 Hz, 1H), 7.39-7.47 (m, 2H), 6.92 (t, 1H), 5.6 (d, 2H), 3.94 (s, 3H). MS(ESI) m/z: 317 [M+1]⁺.

### Embodiment 38. Compound 38-1-Compound 38-4

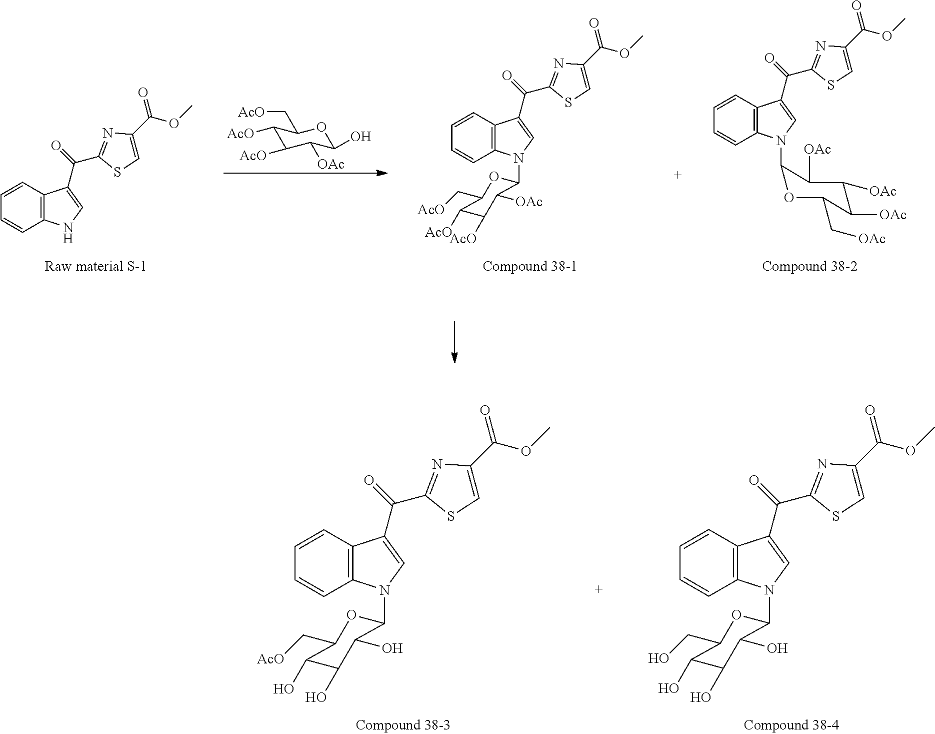 [SMILES: COC(=O)c1csc(C(=O)c2c[nH]c3ccccc23)n1 ; CC(=O)OCC1OC(O)C(OC(C)=O)C(OC(C)=O)C1OC(C)=O ; COC(=O)c1csc(C(=O)c2cn(C3OC(COC(C)=O)C(OC(C)=O)C(OC(C)=O)C3OC(C)=O)c3ccccc23)n1 ; COC(=O)c1csc(C(=O)c2cn(C3OC(COC(C)=O)C(OC(C)=O)C(OC(C)=O)C3OC(C)=O)c3ccccc23)n1 ; COC(=O)c1csc(C(=O)c2cn(C3OC(COC(C)=O)C(O)C(O)C3O)c3ccccc23)n1 ; COC(=O)c1csc(C(=O)c2cn(C3OC(CO)C(O)C(O)C3O)c3ccccc23)n1]

### Synthesis of Compounds 38-1 and 38-2

Raw material SI (1.07 g, 3.78 mmol) was dissolved in THF (50 mL), 2,3,4,6-tetraacetyl glucose (2.6 g, 7.55 mmol) was added therein; and triphenylphosphine (2 g, 7.55 mmol) was added under nitrogen protection. The temperature was reduced to −15° C., and diisopropyl azodicarboxylate (1.53 g, 7.55 mmol) was added dropwise therein. The reaction was poured into ice water, extracted with ethyl acetate (100 mL×2), dried using anhydrous sodium sulfate, and concentrated to dryness under reduced pressure. The residue was purified using silica gel column chromatography (petroleum ether/ethyl acetate: 10/1 to 2/1) to yield compound 38-1 (650 mg) and compound 38-2 (600 mg) (54% yield). Compound 38-1: ¹H NMR (400 MHz, CDCl₃): ¹H NMR (400 MHz, CDCl₃): δ 9.18 (s, 1H), 8.52-8.54 (m, 1H), 8.44 (s, 1H), 7.60-7.63 (m, 1H), 7.38-7.42 (m, 2H), 5.72 (d, J=9.2 Hz, 1H), 5.64 (t, J=9.2 Hz, 1H), 5.50 (t, J=9.6 Hz, 1H), 5.40 (d, J=9.6 Hz, 2H), 4.35 (dd, J=4.8, 12.4 Hz, 2H), 4.27 (dd, J=2 A, 12.4 Hz, 1H), 4.07 (s, 3H), 4.05-4.10 (m, 1H), 2.16 (s, 3H), 2.13 (s, 3H), 2.05 (s, 3H), 1.74 (s, 3H); MS (ESI) m/z: 617.14 [M+11]. Compound 38-2: δ 9.20 (s, 1H), 8.56-8.49 (m, 1H), 8.45 (s, 1H), 7.87-7.80 (m, 1H), 7.44-7.35 (m, 2H), 5.92 (d, J=5.2 Hz, 1H), 5.35 (t, J=2.3 Hz, 1H), 4.99 (dt, J=9.4, 1.7 Hz, 1H), 4.38-4.25 (m, 2H), 4.21-4.12 (m, 2H), 4.04 (s, 3H), 2.21 (s, 3H), 2.18 (s, 3H), 2.16 (s, 3H), 2.07 (s, 3H); MS (ESI) m/z: 617.14 [M+1]⁺.

### Synthesis of Compounds 38-3 and 38-4

Compound 38-1 (200 mg, 0.325 mmol) was dissolved in methanol (10 mL), and sodium methoxide (190 mg, 3.57 mmol) was added therein. The mixture was stirred at room temperature for 5 h. The reaction was poured into a saturated aqueous sodium chloride solution; 50 mL ethyl acetate was added therein; and the pH was adjusted to be neutral with citric acid. The organic phase was separated, and the aqueous phase was extracted again with ethyl acetate. The organic phases were combined, dried using anhydrous sodium sulfate, filtered, and concentrated to dryness under reduced pressure. The residue was purified using silica gel column chromatography (methanol/dichloromethane: 5%-10%) to yield compound 38-3 (40 mg) and compound 38-4 (5 mg). Compound 38-3: MS(ESI) m/z: 491.1 [M+1]⁺. Compound 38-4: MS (ESI) m/z: 449.1 [M+1]⁺.

### Embodiment 39. Compound 39

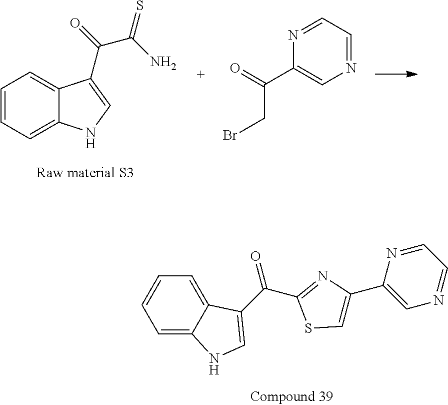 [SMILES: NC(=S)C(=O)c1c[nH]c2ccccc12 ; O=C(CBr)c1cnccn1 ; O=C(c1nc(-c2cnccn2)cs1)c1c[nH]c2ccccc12]

The preparation method was the same as that in Embodiment 21, and compound 39 (65% yield) was prepared. ¹H NMR (400 MHz, DMSO-d₆): δ 12.25 (s, 1H), 9.58 (d, J=0.8 Hz, 1H), 9.38 (d, 3.2 Hz, 1H), 8.79 (s, 1H), 8.76 (d, J=1.2 Hz, 1H), 8.72 (d, J=2.4 Hz, 1H), 8.34-8.36 (m, 1H), 7.60-7.63 (m, 1H), 7.28-7.33 (m, 2H). MS(ESI) m/z: 307 [M+1].

### Embodiment 40. Compound 40

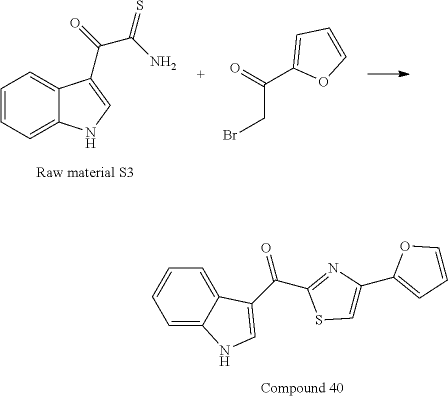 [SMILES: NC(=S)C(=O)c1c[nH]c2ccccc12 ; O=C(CBr)c1ccco1 ; O=C(c1nc(-c2ccco2)cs1)c1c[nH]c2ccccc12]

The preparation method was the same as that in Embodiment 21, and compound 40 (58% yield) was prepared. ¹H NMR (400 MHz, DMSO-d₆): δ 12.29 (s, 1H), 9.23 (d, 3.2 Hz, 1H), 8.33-8.36 (m, 1H), 8.21 (s, 1H), 7.85 (d, J=0.8, 1H), 7.59-7.61 (m, 1H), 7.27-7.33 (m, 2H), 7.13 (d, J=2.8 Hz, 1H), 6.69-6.71 (m, 1H). MS(ESI) m/z: 295 [M+1]⁺.

### Embodiment 41. Compound 41

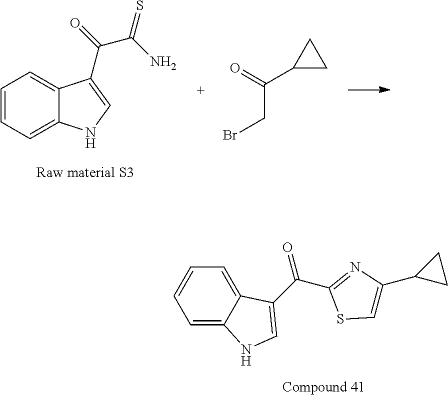 [SMILES: NC(=S)C(=O)c1c[nH]c2ccccc12 ; O=C(CBr)C1CC1 ; O=C(c1nc(C2CC2)cs1)c1c[nH]c2ccccc12]

The preparation method was the same as that in Embodiment 21, and compound 41 (58% yield) was prepared. ¹H NMR (400 MHz, DMSO-d₆): δ 12.19 (s, 1H), 8.99 (d, J=3.2 Hz, 1H), 8.30 (m, 1H), 7.71 (s, 1H), 7.60 (m, 1H), 7.27 (m, 2H), 2.24 (m, 1H), 1.01 (d, J=6.8 Hz, 4H). MS(ESI) m/z′: 269 [M+1]⁺.

### Embodiment 42. Compound 42-1-Compound 42-2

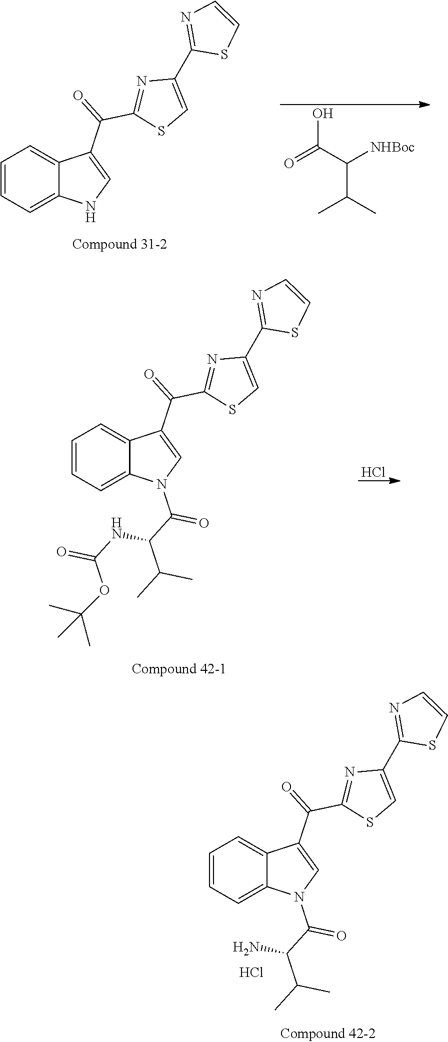 [SMILES: O=C(c1nc(-c2nccs2)cs1)c1c[nH]c2ccccc12 ; CC(C)C(NC(=O)OC(C)(C)C)C(=O)O ; CC(C)[C@H](NC(=O)OC(C)(C)C)C(=O)n1cc(C(=O)c2nc(-c3nccs3)cs2)c2ccccc21 ; Cl ; CC(C)[C@H](N)C(=O)n1cc(C(=O)c2nc(-c3nccs3)cs2)c2ccccc21 ; Cl]

Synthesis method for compound 42-1 was the same as that in Embodiment 4, 83% yield, MS(ESI) m/z: 511.1 [M+1]⁺.

Compound 42-2, 90% yield, ¹H NMR (400 MHz, CDCl₃): δ 9.58 (s, 1H), 9.04 (s, 1H), 8.89 (brs, 3H), 8.78 (m, 1H), 8.46-8.51 (m, 1H), 8.35-8.38 (m, 1H), 8.03 (d, J=3.2 Hz, 1H), 7.96 (d, J=3.2 Hz, 1H), 7.54-7.62 (m, 2H), 5.13 (m, 1H), 2.54-2.59 (m, 1H), 1.15 (d, J=7.2 Hz, 3H), 1.07 (d, J=7.2 Hz, 3H).

MS(ESI) m/z: 411.1 [M+1]⁺.

### Embodiment 43. Compound 43-1-Compound 43-2

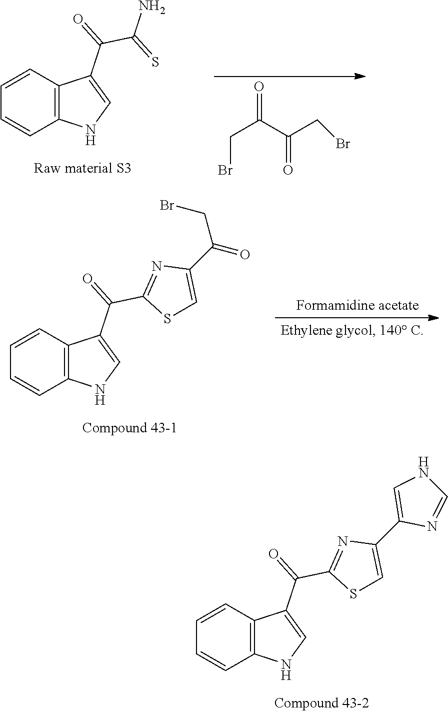 [SMILES: NC(=S)C(=O)c1c[nH]c2ccccc12 ; O=C(CBr)C(=O)CBr ; O=C(CBr)c1csc(C(=O)c2c[nH]c3ccccc23)n1 ; O=C(c1nc(-c2c[nH]cn2)cs1)c1c[nH]c2ccccc12]

Synthesis method for compound 43-1 was the same as that in Embodiment 21, 78% yield, MS (ESI) m/z: 349 [M+1]⁺.

Compound 43-1 (1.8 g, 5.15 mmol) was added to ethylene glycol (35 mL), and formamidine acetate (2.68 g, 25.77 mmol) was added therein. The mixture was reacted at 140° C. (external temperature) under N₂ protection for 2 h. The reaction was cooled, added to ice water, adjusted to pH=9 to 10 with an aqueous sodium hydroxide solution, and extracted with EA. The organic phases were combined and dried. The solvent was removed through distillation under reduced pressure. The solid was washed with a mixture of EA and a small amount of ethanol, and filtered.

The crude product was dissolved with THF, filtered with silica gel, washed with THF, concentrated, and washed with a THF/petroleum ether mixture, and filtered to yield 380 mg of compound 43-2. ¹H NMR (400 MHz, DMSO) δ=12.37 (s, 1H), 12.45 (s, 1H), 9.38 (s, 1H), 8.33-8.38 (m, 1H), 8.04 (s, 1H), 7.79 (s, 2H), 7.58-7.63 (m, 1H), 7.26-7.33 (m, 2H)o MS(ESI) m/z: 295 [M+1]⁺.

### Embodiment 44. Compound 44

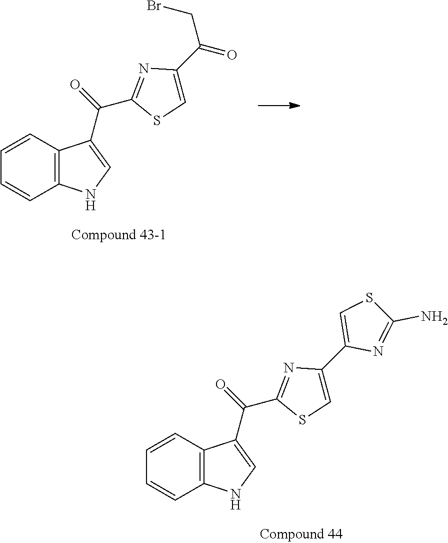 [SMILES: O=C(CBr)c1csc(C(=O)c2c[nH]c3ccccc23)n1 ; Nc1nc(-c2csc(C(=O)c3c[nH]c4ccccc34)n2)cs1]

Compound 43-1 (1.5 g, 4.3 mmol) was dissolved in ethanol (25 mL), and thiourea (327 mg, 4.3 mmol) was added therein. The mixture was reacted at 80° C. for 3 h so that the reaction was complete. The reaction was cooled and filtered. The solid was washed with an aqueous sodium bicarbonate solution, dried, dissolved with THF, and filtered with silica gel. The filtrate was concentrated and then washed with EA to yield 1.2 g of compound 44 (85.6% yield). ¹H NMR (400 MHz, CDCl₃) δ=12.26 (d, J=2.4 Hz, 1H), 9.27 (d, J=3.2 Hz, 1H), 8.32-8.36 (m, 1H), 7.99 (s, 1H), 7.58-7.61 (m, 1H), 7.26-7.32 (m, 3H), 7.21 (s, 2H). MS(ESI) m/z: 327 [M+1]⁺.

### Effect Embodiment 1

AhR agonism assay (refer to the activity determination for agonist MeBio: Oncogene (2004) 23, 4400-4412)

Assay Materials (plasmids): expression of native (Human Hepatoma Huh-7) AhR receptor reporter gene cells, the reporter vector includes functional firefly luciferase gene linked to upstream receptor-specific genetic response elements (GRE).

The AhR agonism assay comprises the following three steps:

1. Cell implantation: a suspension of AhR receptor cells was prepared in cell recovery medium (CRM; FBS containing 10% activated carbon). Then, the prepared suspension (100 μl) was dispensed into wells of a white 96-well culture plate.

2. Right before the beginning of the experiment, Master Stocks were diluted to “2× concentration” treatment media with appropriate compound screening media (CSM: FBS containing 10% activated carbon). The test compounds were subjected to gradient dilution with CSM medium containing 0.2% DMSO, so that the final concentration of DMSO in each experimental well for each treatment group was 0.1%. The treatment media were added to the culture plate on which reporter gene-containing cells had been previously plated (100 uL/well), in duplicate. The experimental plate was placed in an incubator at 37° C. for 24 h.

3. Fluorescence detection and analysis: after completion of the incubation, the treatment media were discarded and 100 μL/well of luciferase detection reagent was added. The Ave RLU (average relative fluorescence intensity) of each well and the coefficient of variation for each experimental group were determined. The activity of AhR receptor with various concentrations of the test compounds was quantitatively determined using a ratio of Ave RLU^{Test Cmpd} of various concentrations of the test compounds in the experimental group to Ave RLU^{Vehicle} of the blank control group, and fold activation and EC₅₀ were determined.

$$Coefficient of variation ( \% CV ) = 100 \times \frac{SD}{Ave RLU} ;$$

$$Fold activation = 100 \times \frac{Ave {RLU}^{Test Cmpd}}{Ave {RLU}^{Vehicle}}$$

For data processing method, refer to J. Biomol. Screen, 1999, 4(2), 67-73.

EC₅₀s of the compounds are shown in Table 1, where A represents 0.001 μM<EC₅₀≤1.0 μM, B represents 1.0 μM<EC₅₀≤10.0 μM, and C represents 10.0 μM<EC₅₀≤100 μM.

TABLE 1
EC₅₀ of each compound

| Compound | EC₅₀ (nM) |
|---|---|
| 1-2 | A |
| 2-2 | A |
| 3 | A |
| 4-2 | A |
| 5 | A |
| 6 | A |
| 7 | A |
| 8 | B |
| 9 | A |
| 10 | C |
| 11 | C |
| 12 | A |
| 13 | B |
| 14 | B |
| 15 | C |
| 16 | A |
| 17 | C |
| 18 | B |
| 19-1 | A |
| 19-2 | A |
| 20-1 | A |
| 20-2 | A |
| 21 | A |
| 22 | A |
| 23 | A |
| 24 | A |
| 25-1 | B |
| 25-2 | B |
| 26-1 | B |
| 26-2 | B |
| 27 | A |
| 28 | C |
| 29 | A |
| 30-1 | A |
| 30-2 | A |
| 31-1 | A |
| 31-2 | A |
| 32-1 | A |
| 32-2 | A |
| 33-1 | A |
| 33-2 | A |
| 34 | B |
| 35 | B |
| 36 | A |
| 37 | A |
| 38-1 | A |
| 38-2 | A |
| 38-3 | A |
| 38-4 | A |
| 39 | A |
| 40 | A |
| 41 | A |
| 42-2 | A |
| 43-2 | A |
| 44 | A |
| 42-1 | A |

Table 1 shows that the compounds described above can bind to AhR and regulate those functions and signaling pathways controlled by AhR, and thus affecting the growth and proliferation of cancer cells and the aggressiveness of tumor cells. Therefore, the pharmaceutical compositions of the compounds of formula (I) of the present invention can be used as AhR inhibitors or non-constitutive AhR agonists to inhibit the growth of cancer cells and inhibit the metastasis and invasion of tumor cells.

### INDUSTRIAL APPLICABILITY

Disclosed in the present invention is an aryl hydrocarbon receptor modulator of formula (I), and a pharmaceutically acceptable salt thereof, wherein

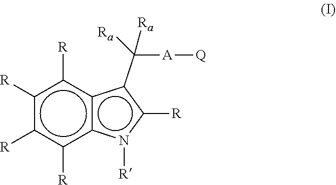

R′ is H, CN, CH₂(OH)R₀, CₘH₂ₘ₊₁, CₙH₂ₙ₋₁, CₙH₂ₙ₋₃,

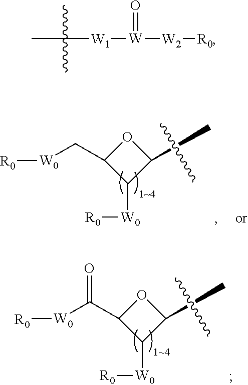

two Rₐ are independently H or two Rₐ together form=0 or ═N—W₃—R₁; A is a C₆ to C₁₀ aromatic ring, a C₂ to C₁₀ heteroaromatic ring interrupted by 1 to 5 heteroatoms selected from N, 0, and S, or a 4 to 7 membered nonaromatic heterocyclic ring containing C═N interrupted by 1 to 3 heteroatoms selected from N, 0, and S, with either one unsubstituted or substituted with 1 to 3 R; Q is R, or is a C₆ to C₁₀ aromatic ring or a C₂ to C₁₀ heteroaromatic ring unsubstituted or substituted with 1 to 3 R and interrupted by 1 to 5 heteroatoms selected from N, 0, and S; and R is R_{C} which is C-attached or R_{N} which is N-attached. The compounds of formula (I) of the present invention can regulate AhR activity, and can be used to inhibit the growth of cancer cells and inhibit the metastasis and invasion of tumor cells.

1. An aryl hydrocarbon receptor modulator of formula (I), and a pharmaceutically acceptable salt thereof, 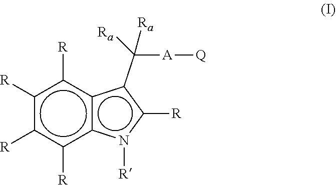
wherein:
R′ is H, CN, CH₂(OH)R₀, CₘH₂ₘ₊₁, CₙH₂ₙ₋₁, CₙH₂ₙ₋₃, 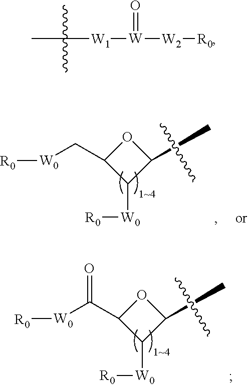
W₀ is O or NH; W₁ is a linker bond, C(R₀)₂, C(R₀)₂O, C(R₀)₂OC(R₀)₂ or C(R₀)₂OC(R₀)₂C(R₀)₂; when W is C, S, or S(O), W₂ is a linker bond, O, NR₀, CH(N(R₀)₂) or OCH₂C(O); when W is P(OR₀), W₂ is O or NR₀; each R₀ is independently H, CₘH₂ₘ₊₁, CₘH₂ₘ₊₁OC(O), CₘH₂ₘ₊₁₋ᵣXᵣ, CₘH₂ₘ₊₁OC(O)CₘH₂ₘ, (cyclic C₄H₈NO)CₘH₂ₘ, CH₃(OCH₂CH₂)ₙ, or CH₃(OCH₂CH₂)ᵤOCH₂;
two Rₐ are independently H or two Rₐ together form ═O, ═N—CN or ═N—W₃—R₁, W₃ is O or NH, R₁ is H, CₘH₂ₘ₊₁, CₘH₂m+1C(O), CₘH₂m+1C(O), or CₘH₂m+1S(O)₁₋₂;
A is a C₆ to C₁₀ aromatic ring unsubstituted or substituted with 1 to 3 R, or a C₂-C₁₀ heteroaromatic ring interrupted by 1 to 5 heteroatoms selected from N, O, and S or a 4 to 7 membered nonaromatic heterocyclic ring containing C═N and interrupted by 1 to 3 heteroatoms selected from N, O, and S, with either one unsubstituted or substituted with 1 to 3 R;
Q is R, or a C₆ to C₁₀ aromatic ring unsubstituted or substituted with 1 to 3 R, or a 3 to 10 membered, preferably 4 to 7 membered, more preferably 5 to 6 membered heterocyclic ring, preferably heteroaromatic ring unsubstituted or substituted with 1 to 3 R, interrupted by 1 to 5, preferably 1 to 3, more preferably 2 to 3 heteroatoms selected from N, O, and S;
R is R_{C} which is C-attached or R_{N} which is N-attached, where each R_{C} is independently X, CN, R″, —Y—OR″, —Y—C(O)R″, —Y—OC(O)R″, —Y—C(O)OR″, —Y—OC(O)OR″, —Y—NR″₂, —Y—C(O)NR″₂, —Y—NR″C(O)R″, —Y—NR″C(O)NR″₂, —Y—OC(O)NR″₂, —Y—NR″C(O)OR″, —Y—S(O)₁₋₂R″, —Y—S(O)₁₋₂NR″₂, or —Y—NR″S(O)₁₋₂R″; each R_{N} is independently CN, R″, —Y—OR″, —Y—C(O)R″, —Y—OC(O)R″, —Y—C(O)OR″, —Y—OC(O)OR″, —Y—NR″₂, —Y—C(O)NR″₂, —Y—NR″C(O)R″, —Y—NR″C(O)NR″₂, —Y—OC(O)NR″₂, —Y—NR″C(O)OR″, —Y—S(O)₁₋₂R″, —Y—S(O)₁₋₂NR″₂, or —Y—NR″ S(O)₁₋₂R″;
R″ is H, D, CₘH₂ₘ₊₁, CₙH₂ₙ₋₁, CₙH₂ₙ₋₃, CₘH₂ₘ₊₁₋ᵣXᵣ, CₙH₂ₙ₋₁₋ₛXₛ, or CₙH₂ₙ₋₃₋ₜXₜ;
Y is a linker bond, —CₘH₂ₘ—, —CₙH₂ₙ₋₂—, —CₙH₂ₙ₋₄—, —CₘH₂ₘ₋ᵢXᵢ—, —CₙH₂ₙ₋₂₋ⱼXⱼ—, or —CₙH₂ₙ₋₄₋ₖXₖ—;
m=1 to 8, n=2 to 8, u=1 to 5, r≤2m+1, s≤2n−1, t≤2n−3, i≤2m, j≤2n−2, k≤2n−4, and X is halogen; preferably, m=1 to 5, more preferably 1 to 3; n=2 to 6, more preferably 2 to 4; u=1 to 4, more preferably 1 to 3; and X is F, Cl, or Br.

2. The aryl hydrocarbon receptor modulator of claim 1, wherein A is 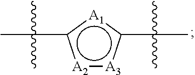
and in which case, formula (1) becomes formula (I1), 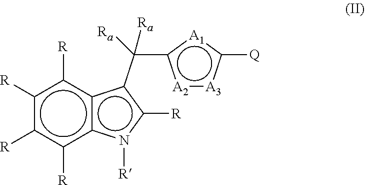
in formula (I1), one of A₁, A₂, and A₃ is O, S, or N(R) and the other two are independently C(R) or N respectively.

3. The aryl hydrocarbon receptor modulator of claim 2, wherein one of A₁, A₂, and A₃ is O, S, or N(R) and the other two are each independently N.

4. The aryl hydrocarbon receptor modulator of claim 3, wherein A₃ is N; and in which case, formula (I1) becomes formula (Ia), 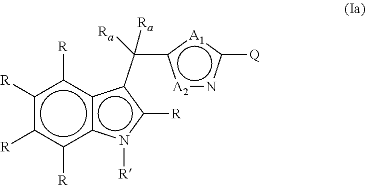
in formula (Ia), A₁ is O, S, or N(R), and A₂ is N; or A₂ is O, S, or N(R), and A₁ is N.

5. The aryl hydrocarbon receptor modulator of claim 2, wherein A₂ is CH; and in which case, formula (I1) becomes formula (Ib), 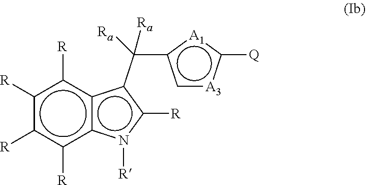
in formula (Ib), A₁ is N or C(R), and A₃ is O, S, or N(R); or A₁ is O, S, or N(R), and A₃ is N or C(R).

6. The aryl hydrocarbon receptor modulator of claim 2, wherein A₁ is N, A₃ is C(R), and two Rₐ together form=N—W₃—R₁; and in which case, formula (I1) becomes formula (Ic), 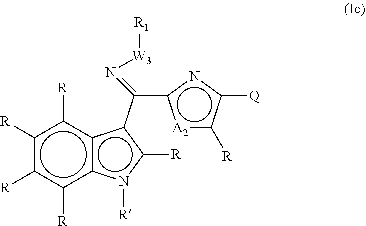
in formula (Ic), A₂ is O, S, or N(R).

7. The aryl hydrocarbon receptor modulator of claim 2, wherein A₁ is N, A₃ is C(R), and two Rₐ are H; and in which case, formula (I1) becomes formula (Id), 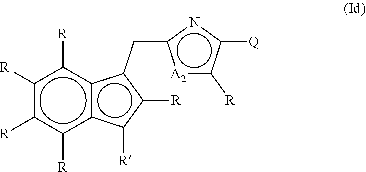
in formula (Id), A₂ is O, S, or N(R).

8. The aryl hydrocarbon receptor modulator of claim 2, wherein A₁ is N, A₃ is C(R), and R′ is 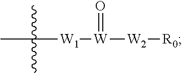
and in which case, formula (I1) becomes formula (Ie), 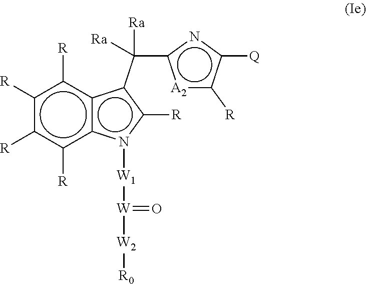
in formula (Ie), A₂ is O, S, or N(R).

9. The aryl hydrocarbon receptor modulator of claim 2, wherein A₁ is N, A₃ is C(R), and R′ is 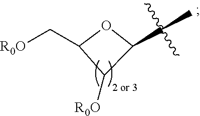
and in which case, formula (II) becomes formula (If), 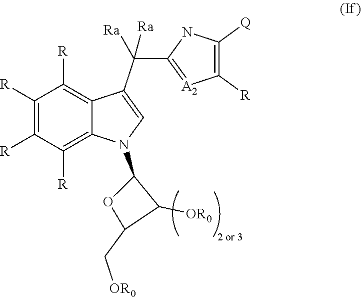
in formula (If), A₂ is O, S, or N(R), and each R₀ is independently H or Ac.

10. The aryl hydrocarbon receptor modulator of claim 1, wherein Q is 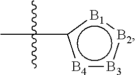
one of B₁, B₂, B₃, and B₄ is O, S, or N(R) and the other three are each independently C(R) or N;
or, Q is 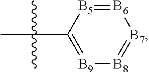
and B₅ to B₉ are C(R); or one or two of B₅ to B₉ are N, and the others are each independently C(R).

11. The aryl hydrocarbon receptor modulator of claim 2, wherein A₁ is N, A₂ is S, A₃ is CH, and Q is a 5 membered heteroaromatic ring; and in which case, formula (I1) becomes formula (Ig), 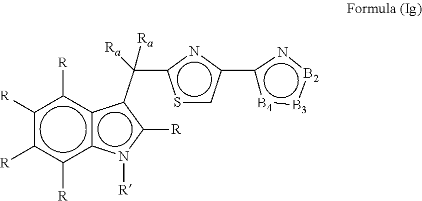
where one of B₂, B₃, and B₄ is O, S, or N(R), and the others are each independently C(R) or N.

12. The aryl hydrocarbon receptor modulator of claim 2, wherein A₁ is N, A₂ is S, A₃ is CH, and Q is a 5 membered nonaromatic heterocyclic ring containing C═N; and in which case, formula (I1) becomes formula (Ih), 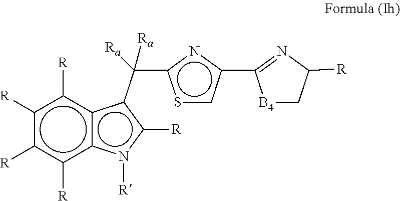
B₄ is O, S, or N(R).

13. The aryl hydrocarbon receptor modulator of claim 1, wherein A is a nonaromatic heterocyclic ring interrupted by N and S, and Q is R; and in which case, formula (I) becomes formula (I2),

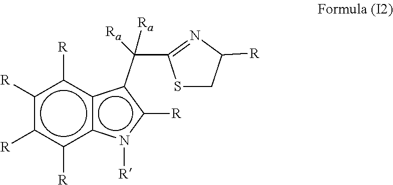

14. The aryl hydrocarbon receptor modulator of claim 1, wherein A is 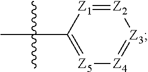
and in which case, formula (I) becomes formula (I3), 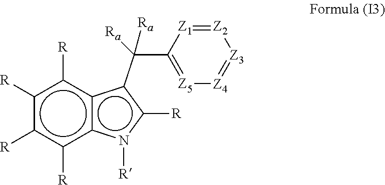
in formula (I3), Z₁ to Z₅ are each independently C(Q); or one or two of Z₁ to Z₅ are N, and the others are each independently C(Q); or adjacent two of Z₁ to Z₅ are C(Q) which together form a 5 to 6 membered carbocyclic ring or a 5 to 6 membered heterocyclic ring interrupted by 1 to 3 heteroatoms selected from N, O, and S, and the other three are each independently C(Q), or two of the other three are each independently C(Q) and the remaining one is N, or one of the other three is C(Q) and the remaining two are N.

15. The aryl hydrocarbon receptor modulator of claim 1, wherein in formula (I), R′ is one of the following substituents:

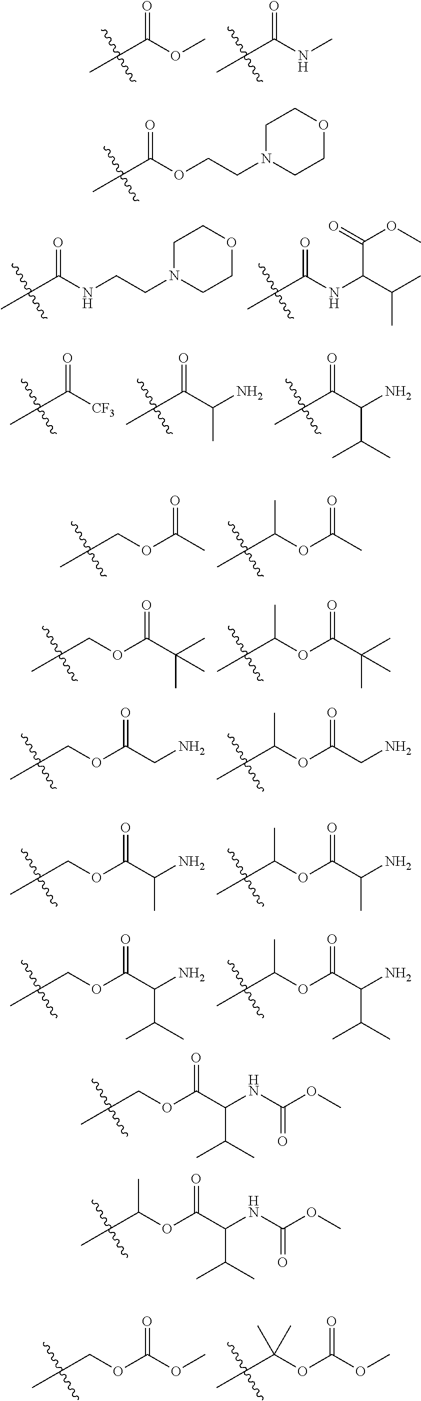

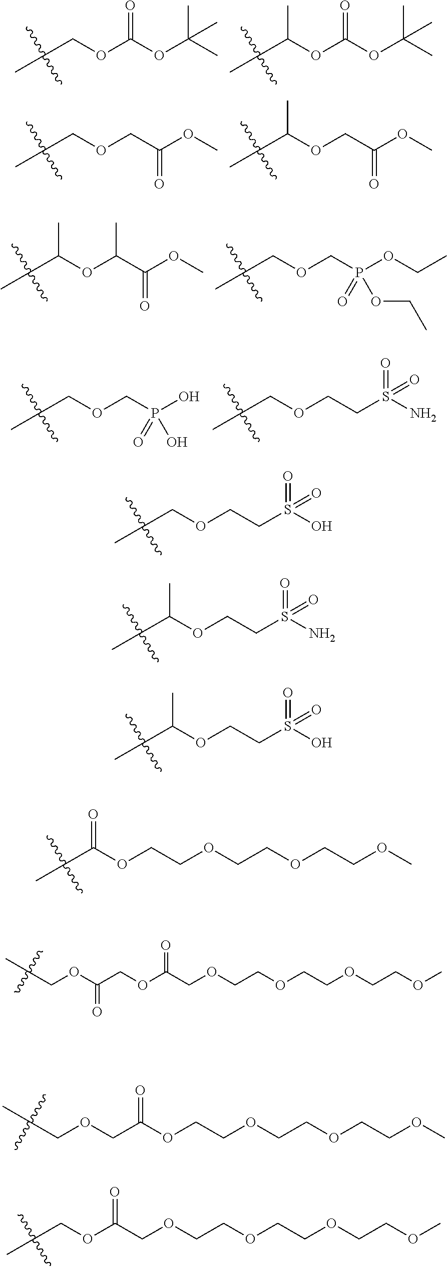

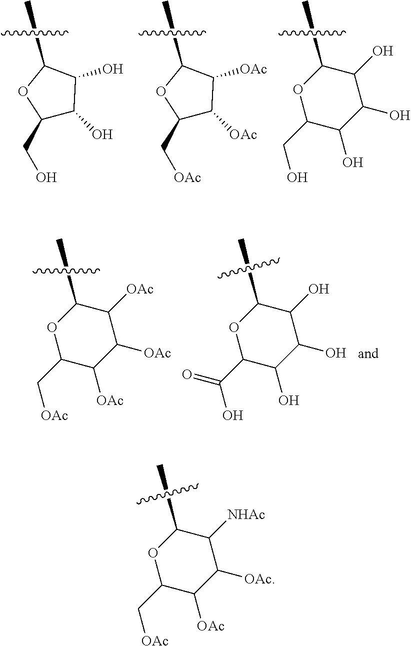

16. The aryl hydrocarbon receptor modulator of claim 3, wherein in formula (I1), 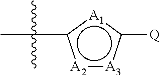
is one of the following substituents:

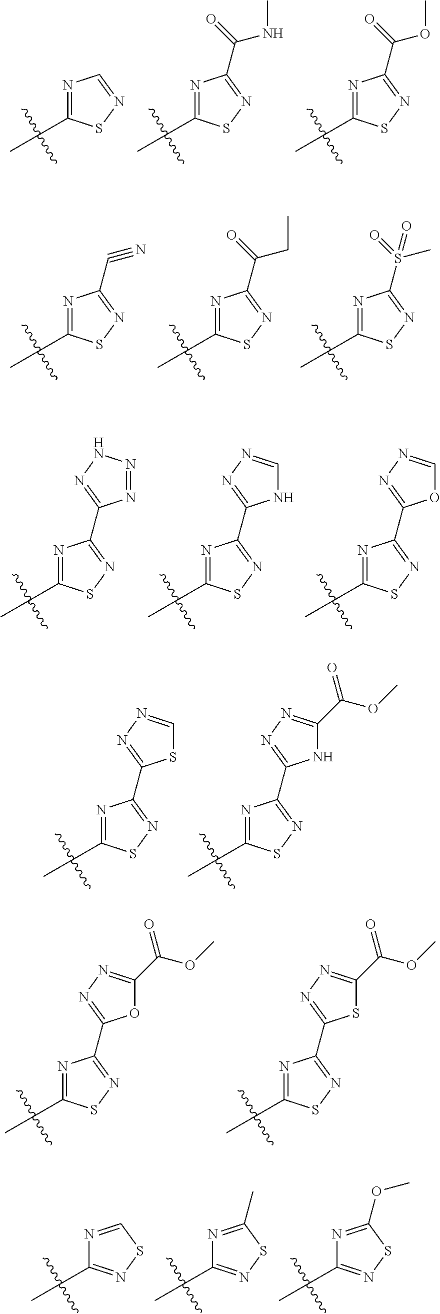

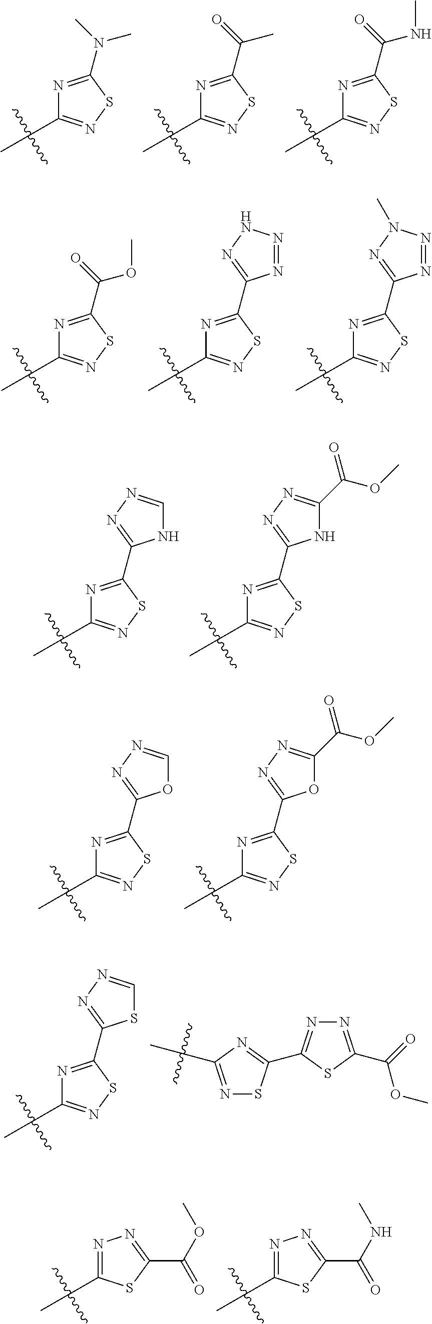

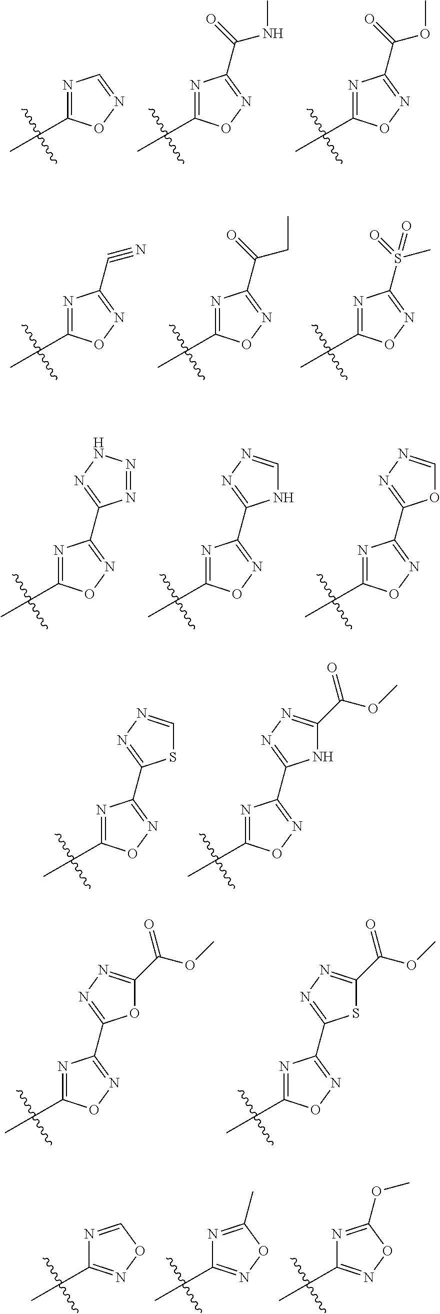

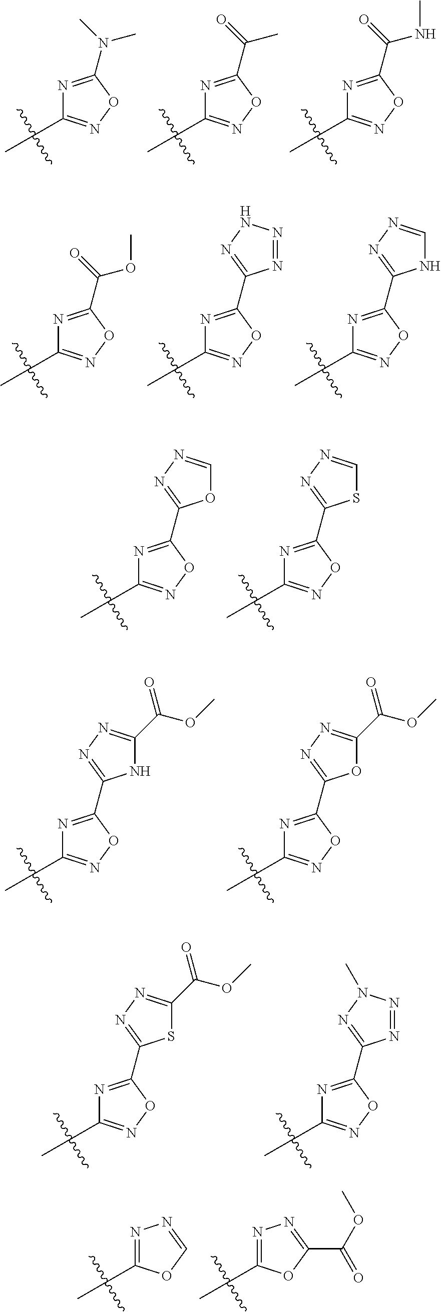

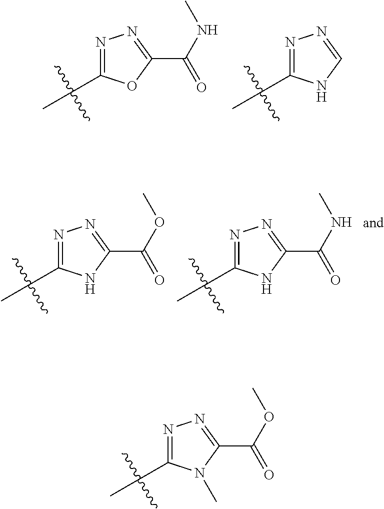

17. The aryl hydrocarbon receptor modulator of claim 5, wherein in formula (Ib), 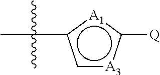
is one of the following substituents:

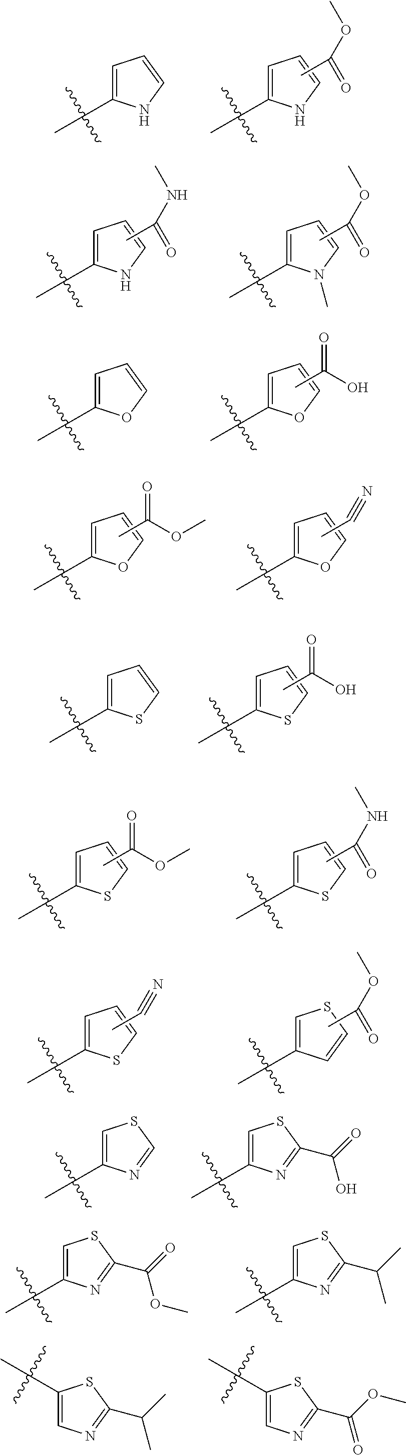

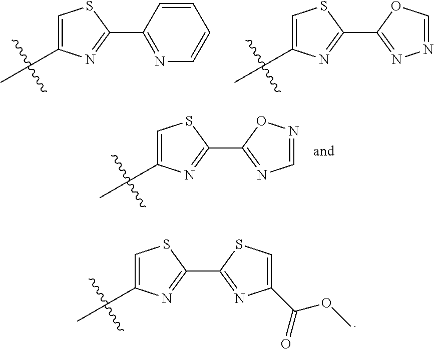 [SMILES: *c1csc(-c2ccccn2)n1 ; *c1csc(-c2nnco2)n1 ; *c1csc(-c2ncno2)n1 ; "and" ; *c1csc(-c2nc(C(=O)OC)cs2)n1]

18. The aryl hydrocarbon receptor modulator of claim 2, wherein in formula (I1), 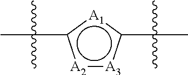
is one of the following substituents:

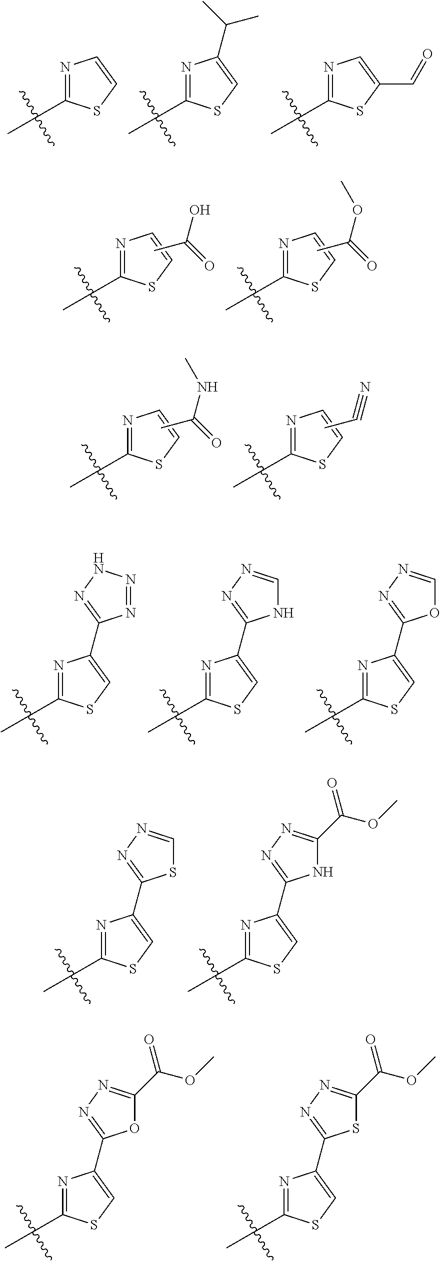

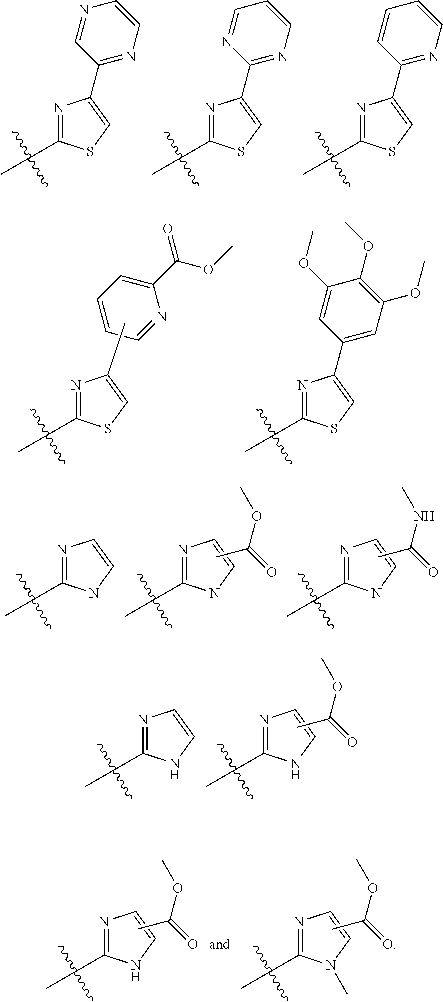

19. The aryl hydrocarbon receptor modulator of claim 1, wherein the aryl hydrocarbon receptor modulator is:

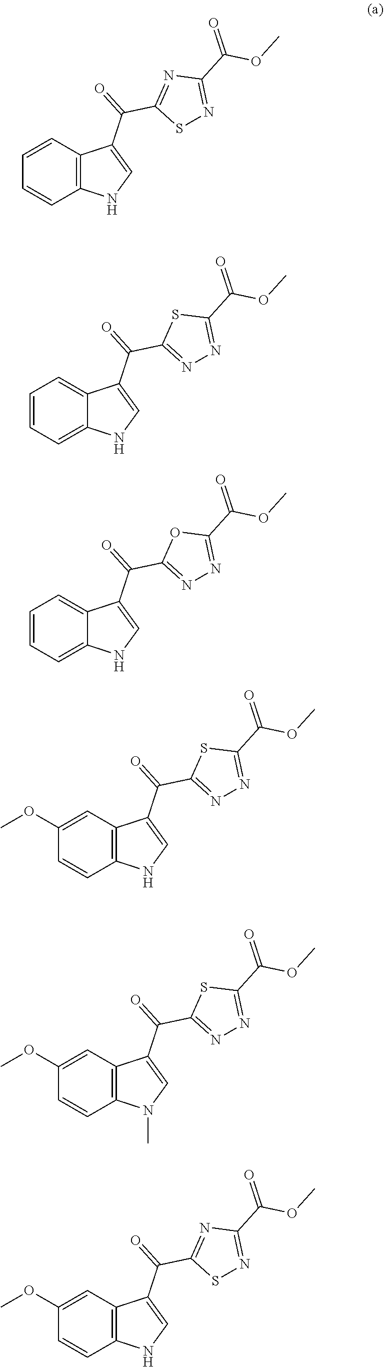

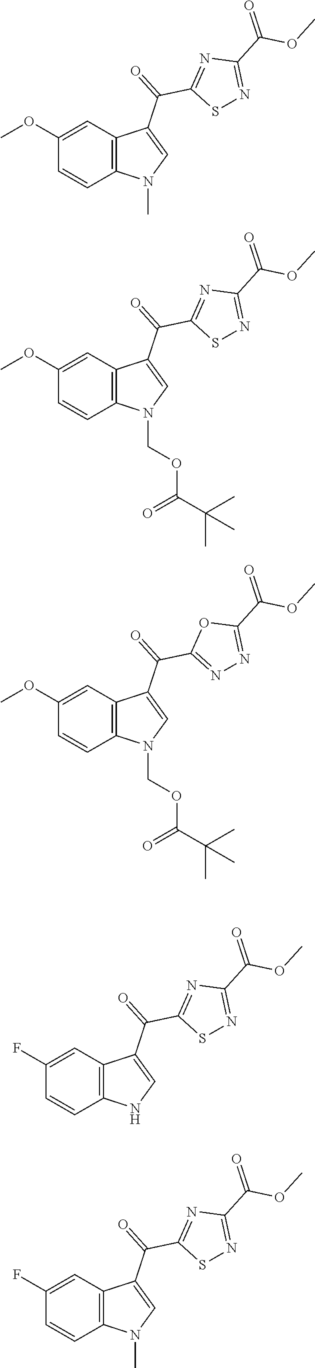

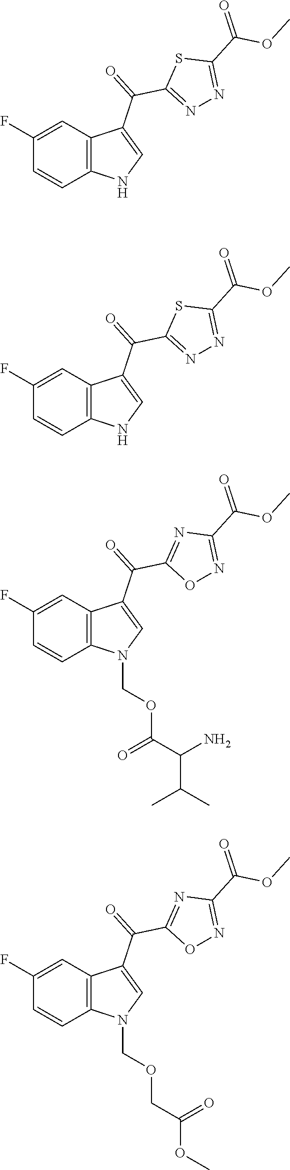

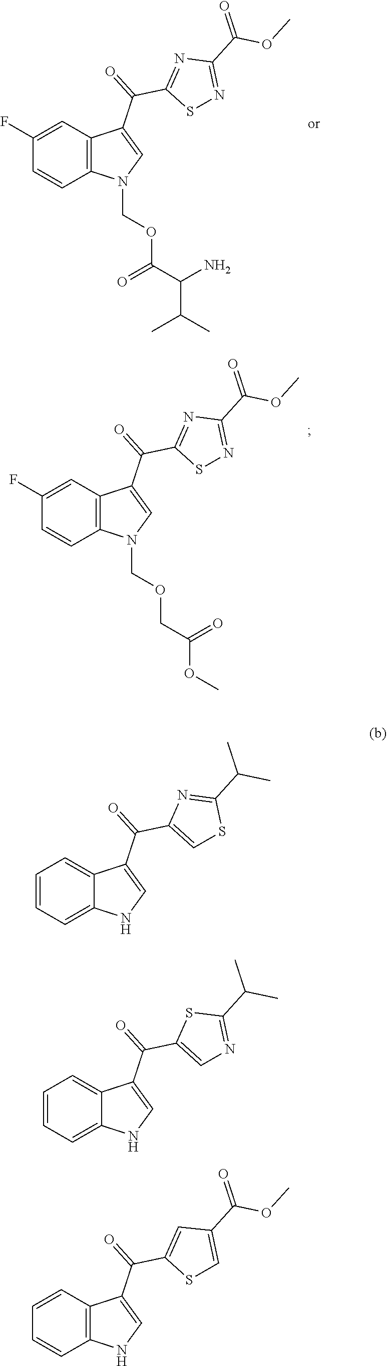

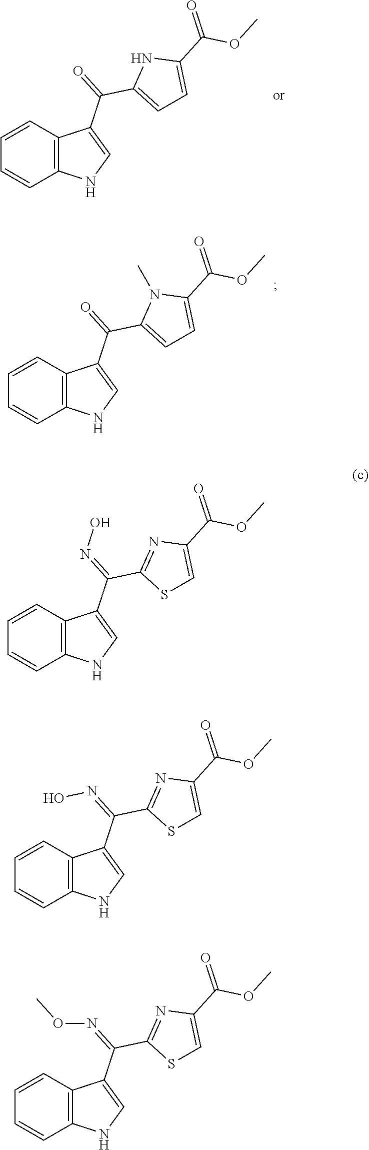 [SMILES: COC(=O)c1ccc(C(=O)c2c[nH]c3ccccc23)[nH]1 ; COC(=O)c1ccc(C(=O)c2c[nH]c3ccccc23)n1C ; COC(=O)c1csc(/C(=N\O)c2c[nH]c3ccccc23)n1 ; COC(=O)c1csc(/C(=N/O)c2c[nH]c3ccccc23)n1 ; CO/N=C(/c1nc(C(=O)OC)cs1)c1c[nH]c2ccccc12]

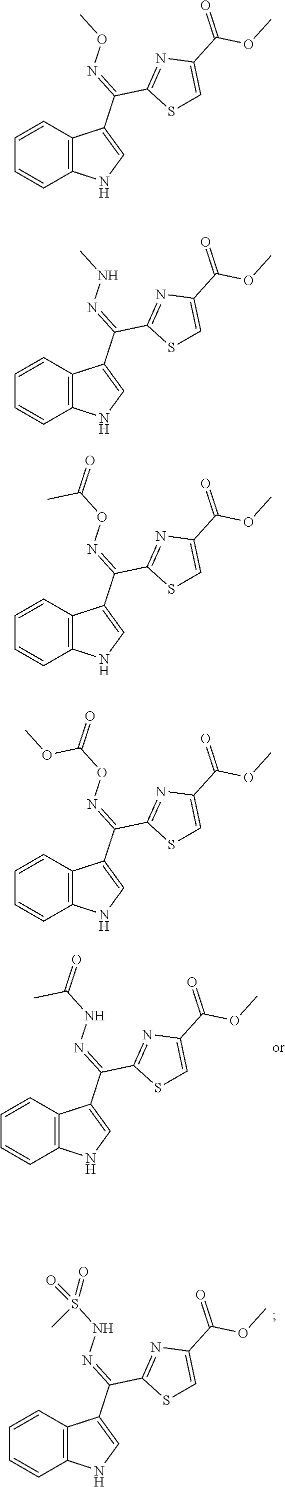

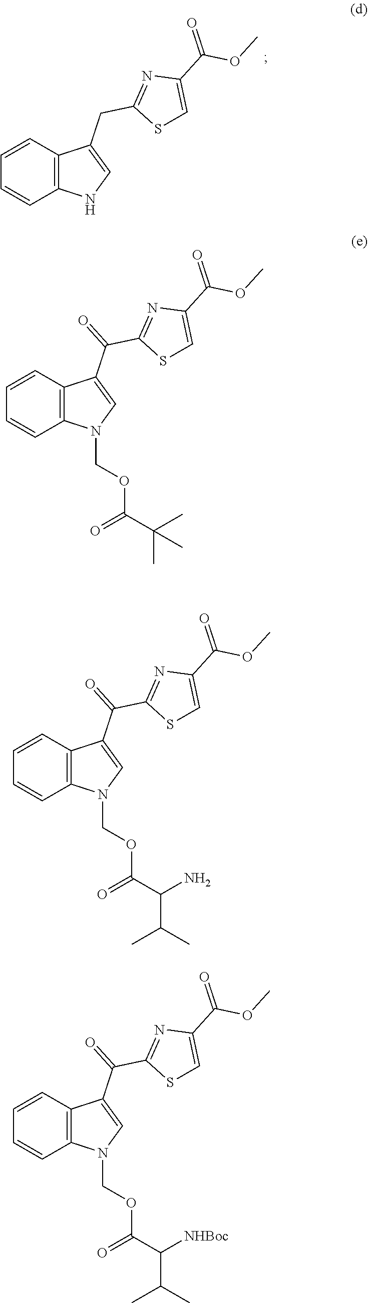

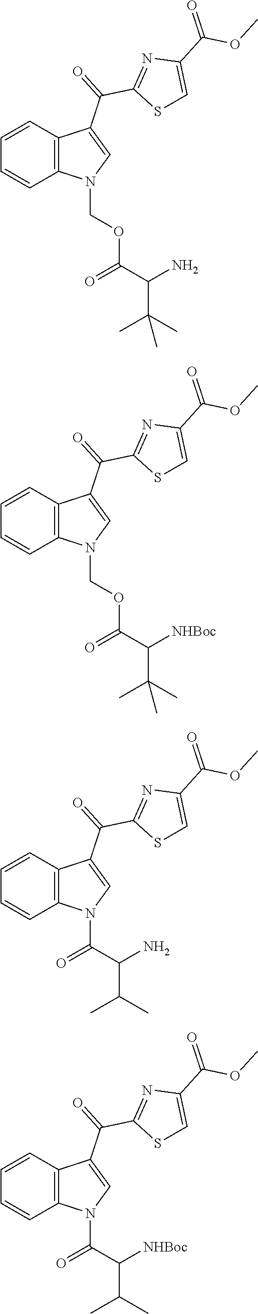

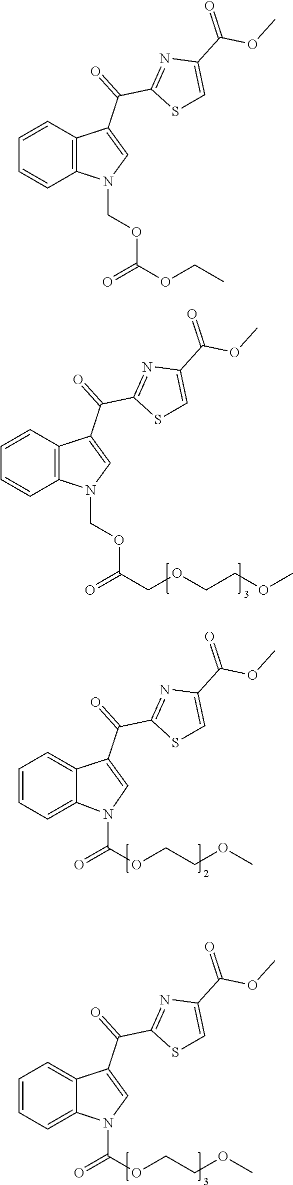

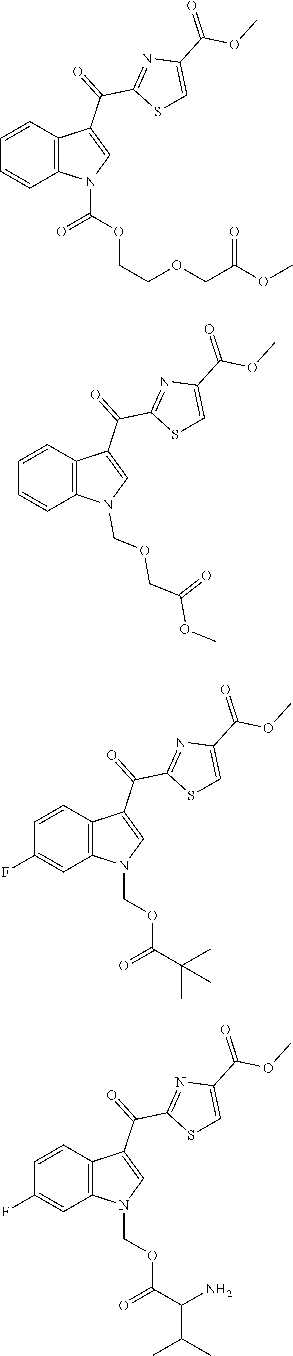 [SMILES: COC(=O)COCCOC(=O)n1cc(C(=O)c2nc(C(=O)OC)cs2)c2ccccc21 ; COC(=O)COCn1cc(C(=O)c2nc(C(=O)OC)cs2)c2ccccc21 ; COC(=O)c1csc(C(=O)c2cn(COC(=O)C(C)(C)C)c3cc(F)ccc23)n1 ; COC(=O)c1csc(C(=O)c2cn(COC(=O)C(N)C(C)C)c3cc(F)ccc23)n1]

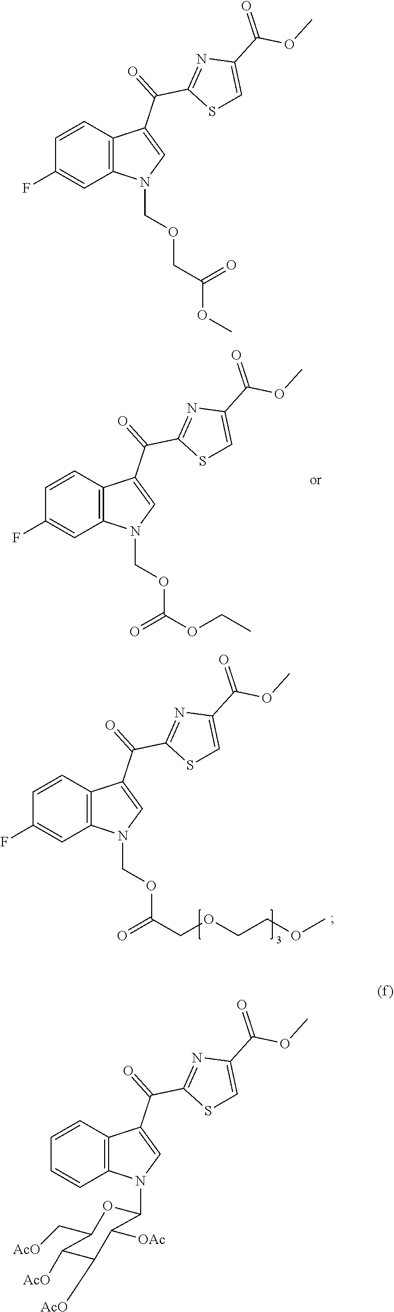

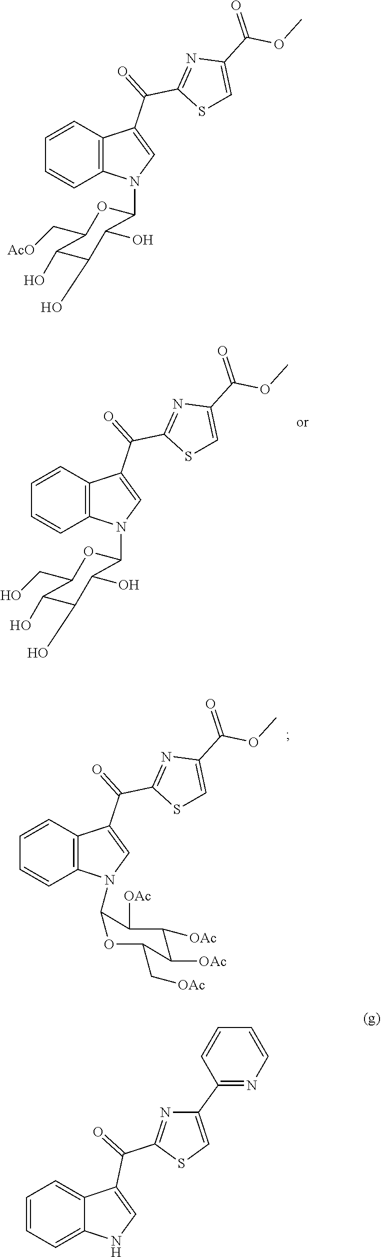 [SMILES: COC(=O)c1csc(C(=O)c2cn(C3OC(COC(C)=O)C(O)C(O)C3O)c3ccccc23)n1 ; COC(=O)c1csc(C(=O)c2cn(C3OC(CO)C(O)C(O)C3O)c3ccccc23)n1 ; COC(=O)c1csc(C(=O)c2cn(C3OC(COC(C)=O)C(OC(C)=O)C(OC(C)=O)C3OC(C)=O)c3ccccc23)n1 ; O=C(c1nc(-c2ccccn2)cs1)c1c[nH]c2ccccc12]

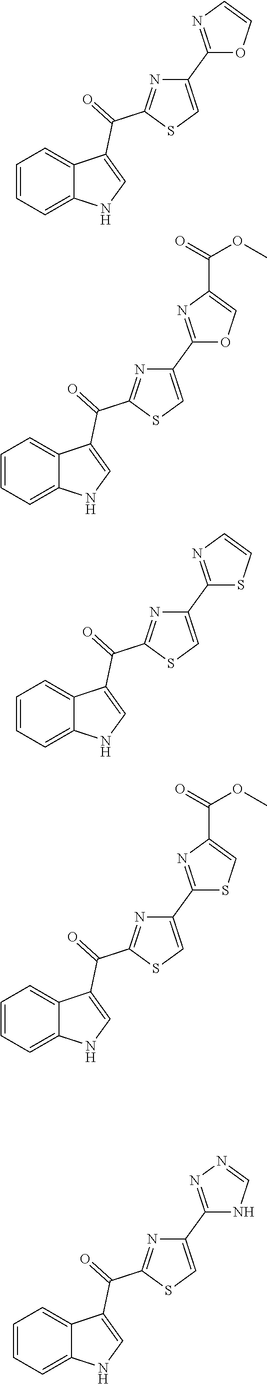 [SMILES: O=C(c1nc(-c2ncco2)cs1)c1c[nH]c2ccccc12 ; COC(=O)c1coc(-c2csc(C(=O)c3c[nH]c4ccccc34)n2)n1 ; O=C(c1nc(-c2nccs2)cs1)c1c[nH]c2ccccc12 ; COC(=O)c1csc(-c2csc(C(=O)c3c[nH]c4ccccc34)n2)n1 ; O=C(c1nc(-c2nnc[nH]2)cs1)c1c[nH]c2ccccc12]

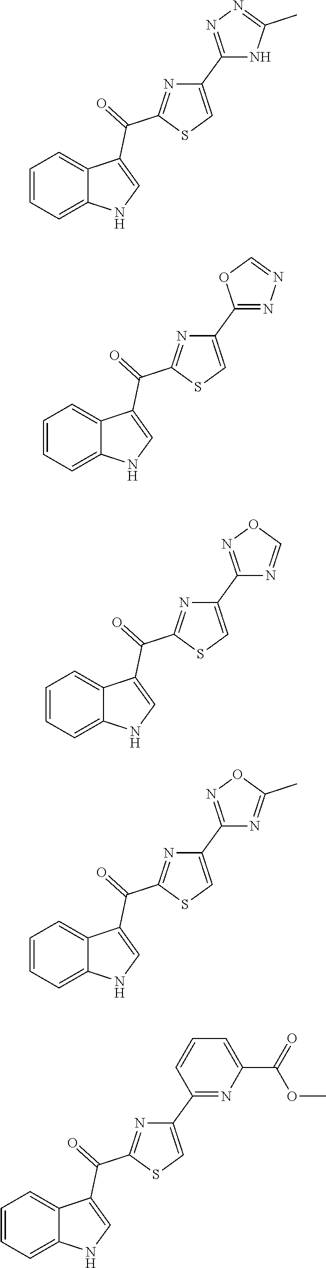 [SMILES: Cc1nnc(-c2csc(C(=O)c3c[nH]c4ccccc34)n2)[nH]1 ; O=C(c1nc(-c2nnco2)cs1)c1c[nH]c2ccccc12 ; O=C(c1nc(-c2ncon2)cs1)c1c[nH]c2ccccc12 ; Cc1nc(-c2csc(C(=O)c3c[nH]c4ccccc34)n2)no1 ; COC(=O)c1cccc(-c2csc(C(=O)c3c[nH]c4ccccc34)n2)n1]

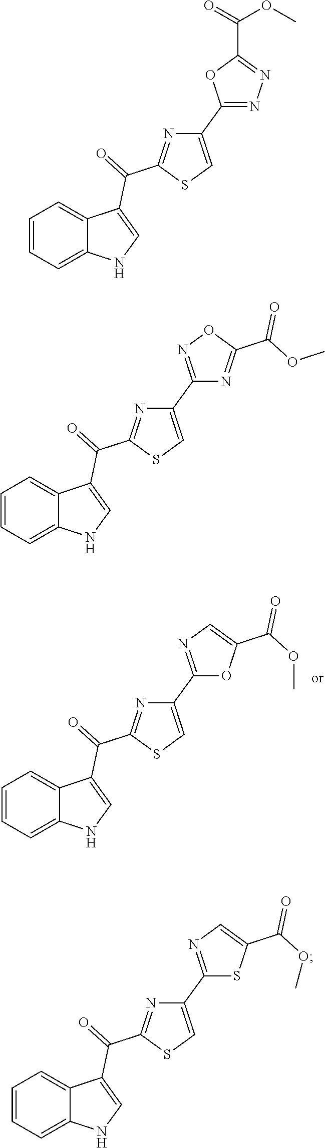 [SMILES: COC(=O)c1nnc(-c2csc(C(=O)c3c[nH]c4ccccc34)n2)o1 ; COC(=O)c1nc(-c2csc(C(=O)c3c[nH]c4ccccc34)n2)no1 ; COC(=O)c1cnc(-c2csc(C(=O)c3c[nH]c4ccccc34)n2)o1 ; COC(=O)c1cnc(-c2csc(C(=O)c3c[nH]c4ccccc34)n2)s1]

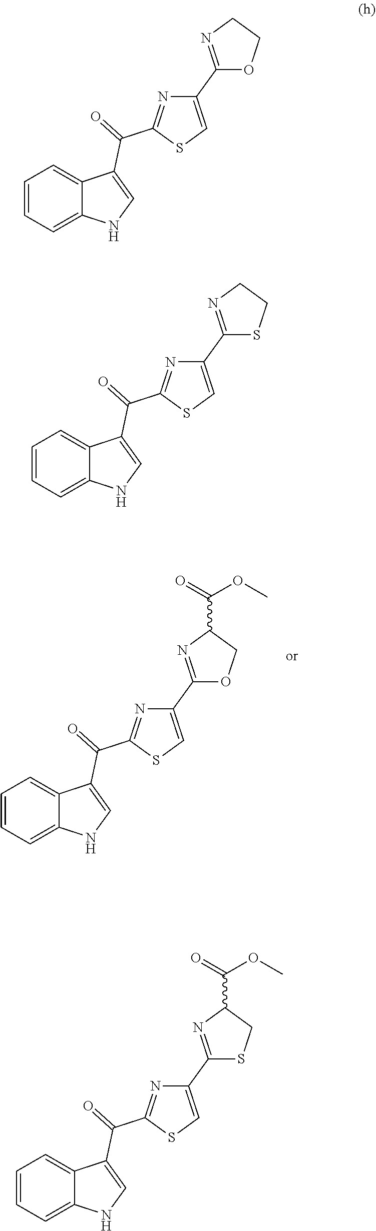 [SMILES: O=C(c1nc(C2=NCCO2)cs1)c1c[nH]c2ccccc12 ; O=C(c1nc(C2=NCCS2)cs1)c1c[nH]c2ccccc12 ; COC(=O)C1COC(c2csc(C(=O)c3c[nH]c4ccccc34)n2)=N1 ; COC(=O)C1CSC(c2csc(C(=O)c3c[nH]c4ccccc34)n2)=N1]

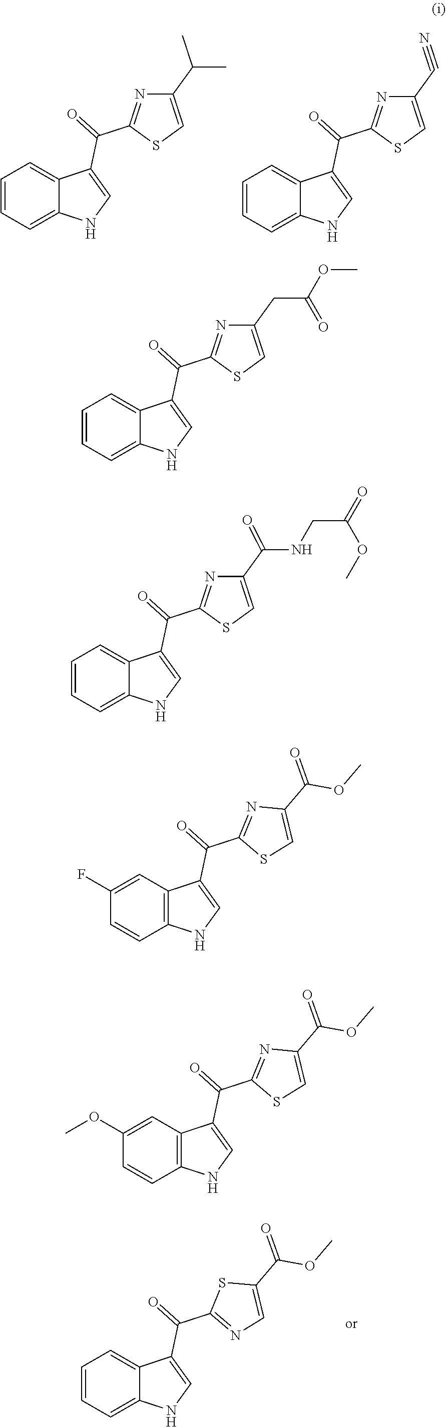

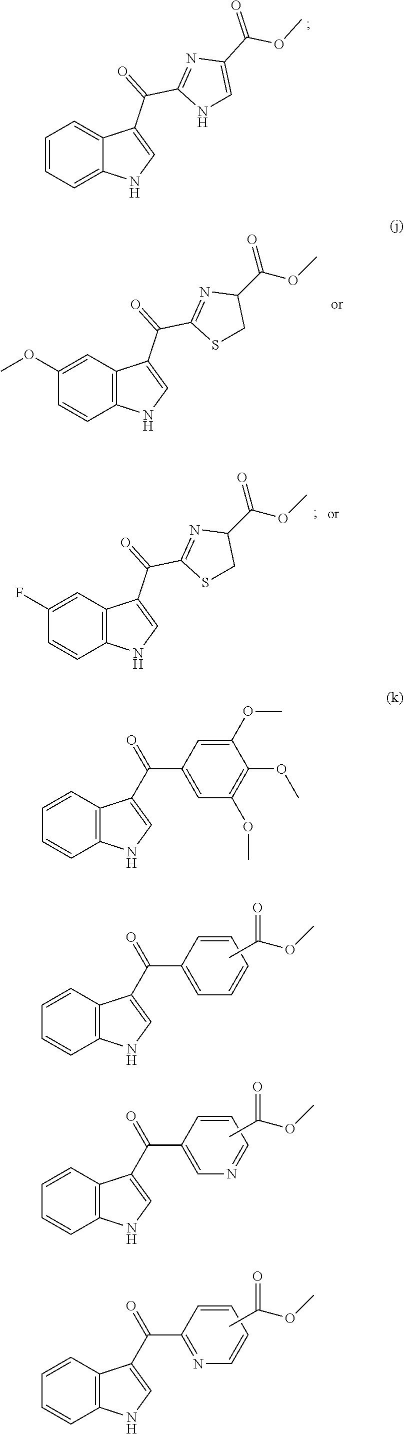

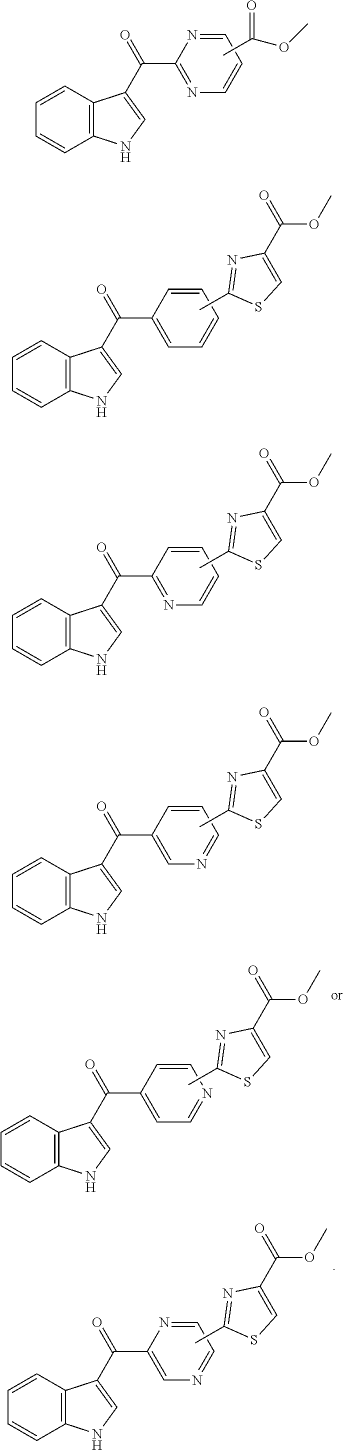

20-29. (canceled)

30. A method of treating cancer in a patient in need thereof, comprising administering a therapeutically effective amount of an aryl hydrocarbon receptor modulator of claim 1 to the patient.
